(19) United States
(12) Patent Application Publication
(10) Pub. No.: US 2020/0071316 A1
(43) Pub. Date: Mar. 5, 2020

(54) ACTIVATORS OF THE RETINOIC ACID INDUCIBLE GENE "RIG-I" PATHWAY AND METHODS OF USE THEREOF

(71) Applicant: Kineta Immuno-Oncology LLC, Seattle, WA (US)

(72) Inventors: Daniel R. Goldberg, Seattle, WA (US); Peter Probst, Seattle, WA (US); Kristin M. Bedard, Bellevue, WA (US)

(21) Appl. No.: 16/536,643

(22) Filed: Aug. 9, 2019

Related U.S. Application Data
(60) Provisional application No. 62/716,830, filed on Aug. 9, 2018.

(51) Int. Cl. C07D 419/02; A61P 35/00; C07D 333/66

(52) U.S. Cl. CPC C07D 419/02; C07D 333/66; A61P 35/00

(57) ABSTRACT

The present invention is directed to compounds of Formula (I), which are activators of the RIG-I pathway.

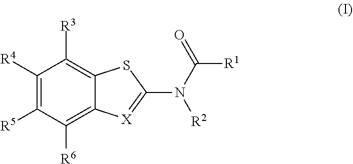

### FIELD OF THE DISCLOSURE

The present invention is directed to compounds and derivatives thereof which are activators of the RIG-I pathway. The present disclosure also relates to the synthesis and to uses of such compounds.

### BACKGROUND OF THE DISCLOSURE

The innate immune system is the first line response against various insults or danger signals including foreign pathogens (e.g., viruses, bacteria and parasites) and cellular damage or abnormalities which may lead to cancer. RIG-I, RIG-I-like receptors (RLRs), Toll-like receptors (TLRs), and the cytosolic DNA receptor, stimulator of interferon genes (STING), are a diverse group of molecules known as pattern-recognition receptors (PRRs). PRRs play a central role in stimulating innate immunity to microbial infections through their ability to recognize pathogen-associated molecular patterns (PAMPs) and signal a cytokine response to control infection. Different PRRs are localized to different cellular compartments, recognize different PAMPs, and signal through different molecular pathways. The common downstream effect is activation of a gene expression program to promote an innate immune response against the invading pathogen. PRRs also play an important role in coordinating the activation and development of the adaptive immune response (Nat Immunol. 2015 April; 16(4):343-353. PMCID: PMC4507498). This includes dendritic cell (DC) recruitment, activation, and antigen presentation to CD8+ T cells. Activation of the transcription factor interferon regulatory factor 3 (IRF3), through RIG-I signaling, is critical for driving DC activation and an antimicrobial response (Immunity. 2014 Nov. 20; 41 (5):830-842. PMCID: PMC4384884).

RIG-I recognizes and is activated by viral RNA PAMPs and by endogenous ligands known as damage-associated molecular patterns (DAMPs) that are released during programmed cell death, stress, or tissue injury. Signaling through activated RIG-I, and the resulting transcription factor IRF-3, leads to the induction of an innate immune response that includes the production of cytokines and chemokines; DC recruitment, activation, and antigen uptake; and the presentation of antigens to CD8+ T cells. RIG-I activation is also associated with immunogenic cell death (ICD), a form of programmed cell death in which an immune response is elicited to antigens derived from dying cells (Nat Rev Immunol. 2017 Feb. 17; 17(2):97-111. PMID: 27748397). ICD is also important to overcome immune tolerance mediated by the tumor microenvironment and to elicit an effective immune response against cancer (Oncoimmunology. 2015 April; 4(4):e1008866. PMCID: PMC4485780). RIG-I is a ubiquitous cytoplasmic protein, and RIG-I RNA is found in all tumor tissues (Vaccine. 2017 Apr. 4; 35(15):1964-1971. PMID: 28279563). Most cancer cells have similar or higher levels of RIG-I protein compared to the level present in normal cells from the same respective tissue and most tumors show moderate to strong cytoplasmic staining for RIG-I by immunohistology (FIG. 2). Interferons and the inflammatory cytokines IL-1β and TNF-α enhance RIG-I expression, whereas the immunosuppressive cytokines IL-10 and TGF-α, abundant in the immune evasive tumor microenvironment, do not control cellular RIG-I levels. Effective immune responses against viruses and tumors share many essential features, and therapeutic benefits of nucleic acid RIG-I ligands (that mimic viral RNA PAMPs) have been demonstrated in several preclinical models of cancer. RIG-I agonists, by inducing ICD and eliciting tumor-targeting T cell populations, may be an effective treatment for cancer, both as a monotherapy or in combination with other cancer immunotherapies. Thus, the use of small-molecule agonists that activate the RIG-I pathway and induce tumor immunity could significantly improve cancer therapies. Accordingly, there is a need for small molecule RIG-I agonists for the treatment of cancer and other diseases. The present invention addresses this and other needs.

### SUMMARY OF THE DISCLOSURE

The present disclosure provides a compound of Formula (I):

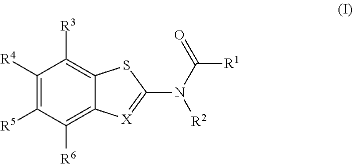

or a pharmaceutically acceptable salt thereof, wherein constituent members are defined herein.

The present disclosure further provides a pharmaceutical composition comprising a compound described herein, or a pharmaceutically acceptable salt thereof, and at least one pharmaceutically acceptable carrier.

The present disclosure further provides a method of activating interferon regulatory factor 3 (IRF3) in an eukaryotic cell, said method comprising contacting a compound described herein, or a pharmaceutically acceptable salt thereof with IRF3 in said eukaryotic cell.

The present disclosure further provides a method of agonizing retinoic acid-inducible gene-I pathway (RIG-I) in an eukaryotic cell, said method comprising contacting a compound described herein, or a pharmaceutically acceptable salt thereof with RIG-I in said eukaryotic cell.

The present disclosure further provides a method of inducing the expression of cytokines that are associated with the RIG-I pathway in an eukaryotic cell, said method comprising contacting a compound described herein, or a pharmaceutically acceptable salt thereof with RIG-I in said eukaryotic cell.

The present disclosure further provides a method of inducing immunogenic cell death in a tumor cell of a subject, said method comprising administering to the subject a therapeutically effective amount of a compound described herein, or a pharmaceutically acceptable salt thereof.

The present disclosure further provides a method for treating a cell-proliferation disorder (e.g., cancer) in a subject, said method comprising administering to the subject a therapeutically effective amount of a compound described herein, or a pharmaceutically acceptable salt thereof.

The present disclosure further provides use of a compound described herein, or a pharmaceutically acceptable salt thereof, in therapy.

The present disclosure further provides a compound described herein, or a pharmaceutically acceptable salt thereof, for use in therapy, such as treating a cell proliferation disorder, for example, cancer. The present disclosure further provides a compound described herein, or a pharmaceutically acceptable salt thereof, for use in the preparation of a medicament for use in therapy, such as treating a cell proliferation disorder, for example, cancer.

### BRIEF DESCRIPTION OF THE DRAWINGS

FIG. 1 shows compound-induced immunogenic cell death in murine colon carcinoma cells. FIG. 1A shows apoptosis of murine colon carcinoma cells expressed as percentage of Annexin V⁺. FIG. 1B shows calreticulin translocation to cell surface, quantified by mean fluorescent intensity (MFI) of calreticulin⁺ live cells (CRT⁺ LDV⁻).

FIG. 2 shows anti-RIG-I immunohistology results using a representative panel of human cancer tissues.

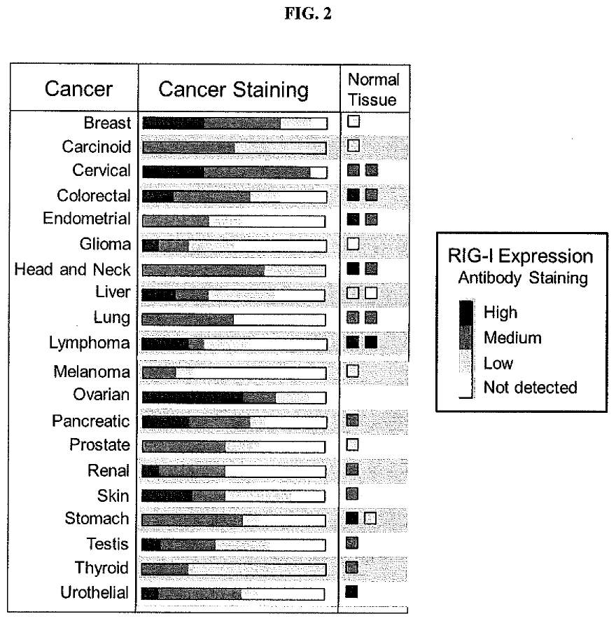

### DETAILED DESCRIPTION OF THE DISCLOSURE

### Compounds

The present disclosure provides a compound of Formula (I):

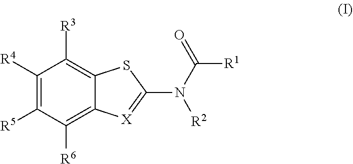

or a pharmaceutically acceptable salt thereof, wherein:

X is N or CR^{X};

R^{X} is H or C₁₋₆ alkyl;

R¹ is a group having Formula (i), (ii), (iii), (iv) or (v):

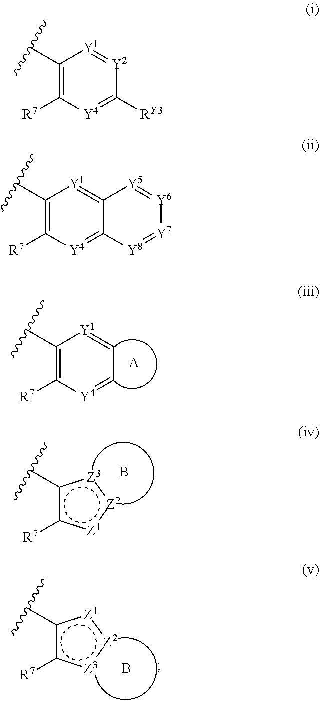

Y¹ is N or CR^{Y1};

Y² is N or CR^{Y2};

Y⁴ is N or CR^{Y4};

Y⁵ is N or CR^{Y5};

Y⁶ is N or CR^{Y6};

Y⁷ is N or CR^{Y7};

Y⁸ is N or CR^{Y8};

wherein not more than four of Y¹, Y², Y⁴, Y⁵, Y⁶, Y⁷, and Y⁸ in formula (ii) are N;

Z¹ is N, CR^{Z1}, O, S, or NR^{Z1};

Z² is N or C;

Z³ is N or C; wherein at least one of Z² and Z³ is N or Z¹ is CR^{Z1};

Ring A is a fused 5-membered heteroaryl group or a fused 4-7 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

Ring B is a fused phenyl, fused C₃₋₇ cycloalkyl, fused 5-6 membered heteroaryl, or fused 4-7 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

the dotted line signifies that the ring containing Z¹, Z² and Z³ is a heteroaroaromatic ring;

R^{Y1}, R^{Y2}, R^{Y4}, R^{Y5}, R^{Y6}, R^{Y7}, R^{Y8}, and R^{Z1} are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{Y1}, R^{Y2}, R^{Y4}, R^{Y5}, R^{Y6}, R^{Y7}, R^{Y9}, and R^{Z1} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{Y3} is phenyl, C₃₋₇ cycloalkyl, 5-6 membered heteroaryl, or 4-7 membered heterocycloalkyl, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}C(S)NR^{c2}R^{d2}, NR^{c2}S(O)R^{b2}, NR^{c2}S(O)₂R^{b2}, NR²S(O)₂NR^{c2}R^{d2}, S(O)R^{b2}, S(O)NR^{c2}R^{d2}, S(O)₂R^{b2}, and S(O)₂NR^{c2}R^{d2};

R² is H or C₁₋₄ alkyl;

R³ is H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R³ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3},NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R⁴ is H, halo, C₁₋₆ alkyl, C₂₋₆ alkenyl, C₂₋₆ alkynyl, C₁₋₆ haloalkyl, C₁₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c1}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R³ are each optionally substituted with 1, 2, 3, 4, or 5 substituents independently selected from Cy⁴, Cy⁴-C₁₋₄ alkyl, halo, C₁₋₆ alkyl, C₂₋₆ alkenyl, C₂₋₆ alkynyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};

wherein at least one of R³ and R⁴ is other than H;

each R⁵ and R⁶ is independently selected from H, halo, C₁₋₆ alkyl, C₁₋₆ haloalkyl, C₆₋₁₀ aryl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a1}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

R⁷ is a group having the formula: —(C₁₋₂ alkyl)ₐ-(L¹)_{b}-(C₂₋₆ alkyl)_{c}-(L²)_{d}-Q;

L¹ is —O—, —S—, —NR⁸—, —CO—, —C(O)O—, —CONR⁸—, —SO—, —SO₂—, —SONR⁸—, —SO₂NR⁸—, or —NR⁸CONR⁹—;

L² is —O—, —S—, —NR¹⁰—, —CO—, —C(O)O—, —CONR¹⁰—, —SO—, —SO₂—, —SONR¹⁰—, —SO₂NR⁸—, or —NR¹⁰CONR⁹—;

R⁸, R⁹, R¹⁰, and R¹¹ are each independently selected from H and C₁₋₄ alkyl;

a is 0 or 1;

b is 0 or 1;

c is 0 or 1;

d is 0 or 1;

wherein the sum of b and d is 1 or 2;

wherein the sum of a and c is 1 or 2;

Q is 5-6 membered heteroaryl or 5-7 membered heterocycloalkyl, each optionally substituted by 1, 2, 3 or 4 substituents selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}CR^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{d3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

each Cy⁴ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c1}R^{d4};

each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{d3} R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, S(O)₂NR^{c6}R^{d6}, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is optionally substituted with 1, 2, 3, 4, or 5 substituents independently selected from Cy⁶, Cy⁶-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c1}C(NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c2} and R^{d2} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6} OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c3} and R^{d3} together with the N atom to which they are attached form a 3-7 membered 3-7 heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c4} and R^{d4} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a5}, R^{b5}, R^{c5}, and R^{d5} is independently selected from H and C₁₋₆ alkyl;

each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and

each R^{c}, R^{c1}, R^{c2}, R^{c3}, R^{c4}, R^{c5}, and R^{c6} is independently selected from H, C₁₋₄ alkyl, and CN,

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group;

wherein one or more ring-forming S atoms of any aforementioned heterocycloalkyl group is optionally substituted by one or two oxo (═O) groups.

In the formula herein, the ring containing Z¹, Z² and Z³ is a heteroaromatic ring. As one skilled in the art understands, for the ring to be heteroaromatic, this ring needs to contain a ring heteroatom, i.e., a ring atom other than carbon. Thus, at least one of Z¹, Z² and Z³ is other than a carbon ring atom. Thus, in the formula, with respect to the ring containing Z¹, Z² and Z³. at least one of is Z² and Z³ is N or Z¹ is N, O, S, or NR^{z1}.

In another embodiment, provided herein is a compound of Formula (I), or a pharmaceutically acceptable salt thereof, wherein:

X is N or CR^{X};

R^{X} is H or C₁₋₆ alkyl;

R¹ is a group having Formula (i), (ii), or (iii):

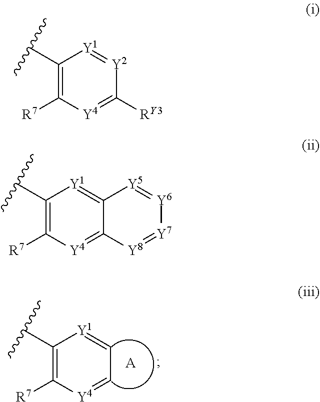

Y¹ is CR^{Y1};

Y² is CR^{Y2};

Y⁴ is CR^{Y4};

Y⁵ is CR^{Y5};

Y⁶ is CR^{Y6};

Y⁷ is CR^{Y7};

Y⁸ is CR^{Y1};

Ring A is a fused 5-membered heteroaryl group or a fused 4-7 membered heterocycloalkyl group, each optionally substituted with 1, 2, 3, 4, or 5 substituents independently selected from Cy¹, halo, C₁₋₆ alkyl, C₁₋₆ haloalkyl, CN, NO₂, OR^{a1}, SR^{a1}, C(O)R^{b1}, C(O)NR^{c1}R^{d1}, and C(O)OR^{a1}, wherein the C₁₋₆ alkyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, S(O)R^{b1}, S(O)₂R^{b1}, and S(O)₂NR^{c1}R^{d1};

R^{Y1}, R^{Y2}, R^{Y4}, R^{Y5}, R^{Y6}, R^{Y7}, R^{Y8}, and R^{Z1} are each independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl of R^{Y1}, R^{Y2}, R^{Y4}, R^{Y5}, R^{Y6}, R^{Y7}, R^{Y8}, and R^{Z1} are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{b1}, OC(O)R^{b1}, OC(O)NR^{c1}R^{d1}, NR^{c1}R^{d1}, NR^{c1}C(O)R^{b1}, S(O)R^{b1}, and S(O)₂R^{b1};

R^{Y3} is phenyl, C₃₋₇ cycloalkyl, 5-6 membered heteroaryl, or 4-7 membered heterocycloalkyl, each optionally substituted with 1, 2, 3, 4, or 5 substituents independently selected from Cy², Cy²-C₁₋₄ alkyl, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, NR^{c2}R^{d2}, NR^{c2}C(O)R^{b2}, S(O)R^{b2}, S(O)NR^{c2}R^{d2}, S(O)₂R^{b2}, and S(O)₂NR^{c2}R^{d2};

R² is H or C₁₋₄ alkyl;

R³ is H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, and C₁₋₆ haloalkyl, of R³ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3},NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R⁴ is H, halo, C₁₋₆ alkyl, C₂₋₆ alkenyl, C₂₋₆ alkynyl, C₁₋₆ haloalkyl, C₆₋₁₀ aryl, C₆₋₁₀ aryl-C₁₋₄ alkyl, CN, NO₂, OR^{a4}, SR^{a4}, C(O)R^{b4}, C(O)NR^{c4}R^{c4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₁₋₆ haloalkyl, C₆₋₁₀ aryl, and C₆₋₁₀ aryl-C₁₋₄ alkyl, of R³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};

wherein at least one of R³ and R⁴ is other than H;

each R⁵ and R⁶ is independently selected from H, halo, C₁₋₆ alkyl, C₁₋₆ haloalkyl, CN, NO₂, OR^{a5}, and SR^{a5};

R⁷ is a group having the formula: —(C₁₋₂ alkyl)ₐ-(L¹)_{b}-(C₂₋₆ alkyl)_{c}-(L²)_{d}-Q;

L¹ is —O—, —S—, —NR⁸—, —CO—, —SO—, —SO₂—, —SO₂NR⁸—, or —SONR⁸—;

L² is —O—, —S—, —NR¹⁰—, —CO—, —SO—, —SO₂—, —SO₂NR⁸—, or —SONR¹⁰—;

R⁸, R⁹, R¹⁰, and R¹¹ are each independently selected from H and C₁₋₄ alkyl;

a is 0 or 1;

b is 0 or 1;

c is 0 or 1;

d is 0 or 1;

wherein the sum of b and d is 1 or 2;

wherein the sum of a and c is 1 or 2;

Q is 5-7 membered heterocycloalkyl, each optionally substituted by 1, 2, 3 or 4 substituents selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, and OC(O)NR^{c}R^{d};

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1};

each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, S(O)R^{b2}, S(O)NR^{c2}R^{d2}, S(O)₂R^{b2}, and S(O)₂NR^{c2}R^{d2};

each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, S(O)R^{b3}, S(O)NR^{c3}R^{d3}, S(O)₂R^{b3}, and S(O)₂NR^{c3}R^{d3};

each R^{a}, R^{b}, R^{c}, R^{d}, R^{a1}, R^{b1}, R^{c1}, R^{d2}, R^{a2}, R^{b2}, R^{c2}, R^{d2}, R^{a3}, R^{b3}, R^{c3}, R^{d3} R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR)NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, S(O)₂NR^{c6}R^{d6}, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)₂R^{b6}, and S(O)₂NR^{c6}R^{d6};

each R^{a5}, R^{b5}, R^{c5}, and R^{d5} is independently selected from H and C₁₋₆ alkyl;

each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄, alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and

each R^{e}, R^{e1}, R^{e2}, R^{e3}, R^{e4}, R^{e5}, and R^{e6} is independently selected from H, C₁₋₄ alkyl, and CN,

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group;

wherein one or more ring-forming S atoms of any aforementioned heterocycloalkyl group is optionally substituted by one or two oxo (═O) groups.

In some embodiments, X is N.

In some embodiments, X is CR^{X}.

In some embodiments, R^{X} is H.

In some embodiments, R¹ is the group having Formula (i):

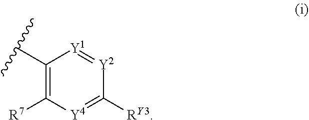

In some embodiments of Formula (i), Y¹ is CR^{Y1}, Y² is CR^{Y2}, and Y⁴ is CR^{Y4}. In some embodiments of Formula (i), R^{Y1}, R^{Y2}, and R^{Y4} are each independently selected from H, halo, C₁₋₆ alkyl, C₁₋₆ haloalkyl, CN, NO₂, and OR^{a1}. In some embodiments of Formula (i), R^{Y1}, R^{Y2}, and R^{Y4} are each H.

In some embodiments of Formula (i), R^{Y3} is phenyl optionally substituted with 1, 2, 3, 4, or 5 substituents independently selected from Cy², Cy²-C₁₋₄ alkyl, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, NR^{c2}R^{d2}, NR^{c2}C(O)R^{b2}, S(O)R^{b2}, S(O)NR^{c2}R^{d2}, S(O)₂R^{b2}, and S(O)₂NR^{c2}R^{d2}. In some embodiments of Formula (i), R^{Y3} is phenyl.

In some embodiments, R¹ is the group having Formula (ii):

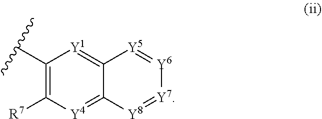

In some embodiments of Formula (ii), Y¹ is CR^{Y1}, Y⁴ is CR^{Y4}, Y⁵ is CR^{Y5}, Y⁶ is CR^{Y6}, Y⁷ is CR^{Y7}, and Y⁸ is CR^{Y8}. In some embodiments of Formula (ii), at least one of Y¹, Y⁴, Y⁵, Y⁶, Y⁷, and Y⁸ is N. In some embodiments, each of R^{Y1}, R^{Y4}, R^{Y5}, R^{Y6}, R^{Y7}, and R^{Y8} are each independently selected from H, halo, C₁₋₆ alkyl, C₁₋₆ haloalkyl, CN, NO₂, and OR^{a1}. In some embodiments of Formula (ii), each of R^{Y1}, R^{Y4}, R^{Y5}, R^{Y6}, R^{Y7}, and R^{Y8} are H.

In some embodiments, R¹ is the group having Formula (iii):

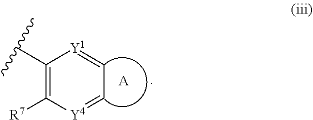

In some embodiments of Formula (iii), Y¹ is CR^{Y1} and Y⁴ is CR^{Y4}. In some embodiments of Formula (iii), R^{Y1} and R^{Y4} are each independently selected from H, halo, C₁₋₆ alkyl, C₁₋₆ haloalkyl, CN, NO₂, and OR^{a1}. In some embodiments of Formula (iii), each of R^{Y1} and R^{Y4} are H.

In some embodiments of the compounds herein, Y¹ is CR^{Y1}, Y⁴ is CR^{Y4}, Y⁵ is CR^{Y5}, Y⁶ is CR^{Y6}, Y⁷ is CR^{Y7}, and Y⁸ is CR^{Y5}. In some embodiments of Formula (ii), at least one of Y¹, Y⁴, Y⁵, Y⁶, Y⁷, and Y⁸ is N. In some embodiments, each of R^{Y1}, R^{Y4}, R^{Y5}, R^{Y6}, R^{Y7}, and R^{Y8} are each independently selected from H, halo, C₁₋₆ alkyl, C₁₋₆ haloalkyl, CN, NO₂, and OR^{a1}. In some embodiments of Formula (ii), each of R^{Y1}, R^{Y4}, R^{Y5}, R^{Y6}, R^{Y7}, and R^{Y8} are H.

In some embodiments of Formula (iii), A is a fused 5-membered heteroaryl group optionally substituted with C₁₋₆ alkyl. In some embodiments of Formula (iii), A is a fused 4-7 membered heterocycloalkyl group, optionally substituted with C₁₋₆ alkyl. In some embodiments of Formula (iii), A is pyrrolyl, thiophenyl, or 1,3-dioxonyl, each of which is optionally substituted with methyl.

In some embodiments, R¹ is the group having Formula (iv):

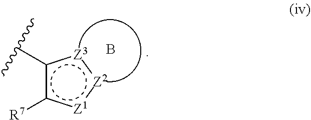

In some embodiments of Formula (iv), Z¹ is NR^{Z1}, O, or S. In some embodiments of Formula (iv), Z¹ is NR^{Z1}. In some embodiments, Z¹ is O. In some embodiments of Formula (iv), Z¹ is S.

In some embodiments of Formula (iv), Z² is C.

In some embodiments of Formula (iv), Z³ is C.

In some embodiments of Formula (iv), Ring B is a fused phenyl or fused 5-6 membered heteroaryl, each optionally substituted with 1, 2, 3, 4, or 5 substituents independently selected from halo, C₁₋₆ alkyl, C₁₋₆ haloalkyl, CN, NO₂, and OR^{a1}.

In some embodiments of Formula (iv), Ring B is a fused phenyl.

In some embodiments, R¹ is the group having Formula (v):

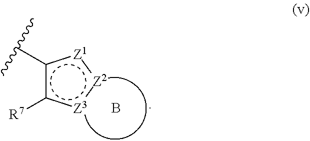

In some embodiments of Formula (v), Z¹ is NR^{Z1}, O, or S. In some embodiments of Formula (v), Z¹ is NR^{Z1}. In some embodiments, Z¹ is O. In some embodiments of Formula (v), Z¹ is S.

In some embodiments of Formula (v), Z² is C.

In some embodiments of Formula (v), Z³ is C.

In some embodiments of Formula (v), Ring B is a fused phenyl or fused 5-6 membered heteroaryl, each optionally substituted with 1, 2, 3, 4, or 5 substituents independently selected from halo, C₁₋₆ alkyl, C₁₋₆ haloalkyl, CN, NO₂, and OR^{a1}.

In some embodiments of Formula (v), Ring B is a fused phenyl.

In some embodiments, R³ is H or NR^{c3}C(S)NR^{c3}R^{d3}. In some embodiments, R³ is H. In some embodiments, R³ is NR^{c3}C(S)NR^{c3}R^{d3}, In some embodiments, R³ is other than H.

In some embodiments, each R³ is selected from H and C₆₋₁₀ aryl, wherein said C₆₋₁₀ aryl is optionally substituted with 1, 2, 3, 4, or 5 substituents independently selected from halo, C₁₋₄ alkyl, C₁₋₆ haloalkyl, CN, OR^{a6}, and NR^{c6}R^{d6}. In some embodiments, each R^{c3} is selected from H and phenyl. In some embodiments, R^{a3} is selected from H and C₆₋₁₀ aryl, wherein said C₆₋₁₀ aryl is optionally substituted with 1, 2, 3, 4, or 5 substituents independently selected from halo, C₁₋₄ alkyl, C₁₋₆ haloalkyl, CN, OR^{a6}, and NR^{c6}R^{d6}. In some embodiments, each R³ is selected from H and phenyl.

In some embodiments, R⁴ is H, halo, C₁₋₆ alkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, C(O)NR^{c4}R^{d4}, S(O)₂R^{b4}, C₆₋₁₀ aryl-C₁₋₄ alkyl, NO₂, NR^{c4}R^{d4}, NR^{c4}C(O)R^{b4}, CN, NR^{c4}S(O)₂R^{b4}, or C(O)OR^{a4}.

In some embodiments, R⁴ is halo, C₁₋₆ alkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, C(O)NR^{c4}R^{d4}, S(O)₂R^{b4}, C₆₋₁₀ aryl-C₁₋₄ alkyl, NO₂, NR^{c4}R^{d4}, NR^{c4}C(O)R^{b4}, CN, NR^{c4}S(O)₂R^{b4}, or C(O)OR^{a4}.

In some embodiments, R⁴ is H, C(O)NR^{c4}R^{d4}, S(O)₂R^{b4}, benzyl, NO₂, NR^{c4}R^{d4}, NR^{c4}C(O)R^{b4}, CN, NR^{c4}S(O)₂R^{b4}, or C(O)OR^{a4}.

In some embodiments, R⁴ is C(O)NR^{c4}R^{d4}, S(O)₂R^{b4}, benzyl, NO₂, NR^{c4}R^{d4}, NR^{c4}C(O)R^{b4}, CN, NR^{c4}S(O)₂R^{b4}, or C(O)OR^{a4}.

In some embodiments, R⁴ is other than H.

In some embodiments, each R^{a4} is H or C₁₋₆ alkyl. In some embodiments, each R^{a4} is H or methyl.

In some embodiments, each R^{b4} is independently selected from H, halo, C₁₋₆ alkyl, C₁₋₆ haloalkyl, C₃₋₇ cycloalkyl, 4-10 membered heterocycloalkyl, OR⁶, NR^{c6}R^{d6}, SR^{a6}, wherein said C₁₋₆ alkyl, C₃₋₇ cycloalkyl, and 4-10 membered heterocycloalkyl is optionally substituted with halo, C₁₋₄ alkyl, C₁₋₄ haloalkyl, OR^{a6}, SR^{a6}, or NR^{c6}R^{d6}. In some embodiments, each R^{b4} is methyl, N(CH₃)₂, SCHF₂, OCH₂CF₃, phenyl, morpholinyl, cyclohexyl, 2-oxa-6-azaspiro[3.3]heptanyl, pyrrolidinyl, azetidinyl, or piperidinyl; wherein said morpholinyl, cyclohexyl, pyrrolidinyl, azetidinyl, and piperidinyl are each optionally substituted with alkyl or OR^{a6}.

In some embodiments, R^{c4} is H, C₁₋₆ alkyl, C₁₋₆ haloalkyl, C₃₋₇ cycloalkyl, C₆₋₁₀ aryl, 5-10 membered heteroaryl, wherein each R^{c4} is optionally substituted with 1, 2, 3, 4, or 5 substituents independently selected from halo, C₁₋₄ alkyl, C₁₋₄ haloalkyl, and C₁₋₆ haloalkyl. In some embodiments, R^{c4} is H, cyclopropyl, pyridinyl, or phenyl, wherein said pyridinyl and phenyl are each optionally substituted with 1, 2, or 3 substituents independently selected from halo and C₁₋₄ alkyl.

In some embodiments, R^{d4} is H, C₁₋₆ alkyl, C₁₋₆ haloalkyl, C₃₋₇ cycloalkyl, C₆₋₁₀ aryl, 5-10 membered heteroaryl, wherein each R^{d4} is optionally substituted with 1, 2, 3, 4, or 5 substituents independently selected from halo, C₁₋₄ alkyl, C₁₋₄ haloalkyl, and C₁₋₆ haloalkyl. In some embodiments, R^{d4} is H, methyl, cyclopropyl, pyridinyl, or phenyl, wherein said pyridinyl and phenyl are each optionally substituted with 1, 2, or 3 substituents independently selected from halo and C₁₋₄ alkyl.

In some embodiments, R² is H. In some embodiments, R² is C₁₋₄ alkyl. In some embodiments, R² is methyl.

In some embodiments, R⁵ is H.

In some embodiments, R⁵ is H, halo, C₁₋₆ alkyl, C₁₋₆ haloalkyl, CN, NO₂, OR^{a5}, or NR^{c5}SR^{d5}. In some embodiments, R⁵ is H, halo, or C₁₋₆ alkyl.

In some embodiments, R⁶ is H.

In some embodiments, R⁶ is H, halo, C₁₋₆ alkyl, C₁₋₆ haloalkyl, CN, NO₂, OR^{a5}, or NR^{c5}R^{d5}. In some embodiments, R⁶ is H, halo, or C₁₋₆ alkyl.

In some embodiment, both R⁵ and R⁶ are H.

In some embodiments, a is 0.

In some embodiments, a is 1.

In some embodiments, b is 0.

In some embodiments, b is 1.

In some embodiments, c is 0.

In some embodiments, c is 1.

In some embodiments, d is 0.

In some embodiments, d is 1.

In some embodiments, a is 0, b is 1, c is 1, and d is 0.

In some embodiments, L¹ is —O—, —NR⁸—, —CO—, —C(O)O—, or —CONR⁸—. In some embodiments, L¹ is —O—.

In some embodiments, Q is 5-7 membered heterocycloalkyl, optionally substituted by 1, 2, 3 or 4 substituents selected from halo, C₁₋₆ alkyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, C(O)R^{b}, C(O)NR^{c}R^{d}, C(O)OR^{a}, OC(O)R^{b}, NR^{c}R^{d}, NR^{c}C(O)R^{b}, S(O)R^{b}, S(O)NR^{c}R^{d}, S(O)₂R^{b}, and S(O)₂NR^{c}R^{d}.

In some embodiments, Q is 5-7 membered heterocycloalkyl optionally substituted with halo. In some embodiments, Q is morpholinyl or piperidinyl, each optionally substituted with halo. In some embodiments, Q is morpholinyl. In some embodiments, Q is piperidinyl optionally substituted with halo.

In some embodiments, R⁷ is a group having the formula:

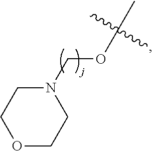

wherein j is 2, 3, 4, 5, or 6.

In some embodiments, R¹ is of Formula (i), (ii), (iii), (iv), or (v), R⁷ is a group having the formula:

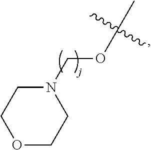

R⁴ is C(O)NR^{c4}R^{d4}, S(O)₂R^{b4}, benzyl, NO₂, NR^{c4}R^{d4}, NR^{c1}C(O)R^{b4}, CN, or C(O)OR^{a4}S(O)₂R^{b4}. In some embodiments, R⁴ is S(O)₂R^{b4} and R^{b4} is a 4-10 membered heterocycloalkyl, such as pyrrolidinyl, morpholinyl, azetidinyl, or piperidinyl; wherein said pyrrolidinyl, azetidinyl, morpholinyl, and piperidinyl are each optionally substituted with halo, C₁₋₄ alkyl, C₁₋₄ haloalkyl, OR^{a6}, SR^{a6}, or NR^{c6}R^{d6}. In some embodiments thereof, R³ is hydrogen, halo, C₁₋₄ haloalkyl, C₁₋₄ alkyl, or NR^{c3}C(S)NR^{c3}R^{d3}. In another embodiment, R¹ is of Formula (ii), R⁷ is a group having the formula:

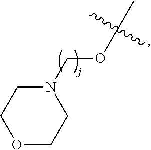

R⁴ is C(O)NR^{c4}R^{d4}, S(O)₂R^{b4}, benzyl, NO₂, NR^{c4}R^{d4}, NR^{c4}C(O)R^{b4}, CN, or C(O)OR^{a4}S(O)₂R^{b4}. In some embodiments, R⁴ is S(O)₂R^{b4}, and R^{b4} is a 4-10 membered heterocycloalkyl, such as pyrrolidinyl, morpholinyl, azetidinyl, or piperidinyl; wherein said, pyrrolidinyl, azetidinyl, morpholinyl, and piperidinyl are each optionally substituted with halo, C₁₋₄ alkyl, C₁₋₄ haloalkyl, OR^{a6}, SR^{a6}, or NR^{c6}R^{d6}. In some embodiments thereof, R³ is hydrogen, halo, C₁₋₄ haloalkyl, C₁₋₄ alkyl, or NR^{c3}C(S)NR^{c3}R^{d3}.

In some embodiments, provided herein is a compound having Formula (II):

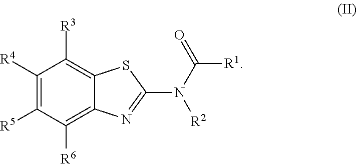

In some embodiments, provided herein is a compound having Formula (IIa):

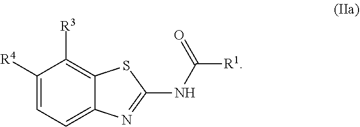

In some embodiments, provided herein is a compound having Formula (IIb):

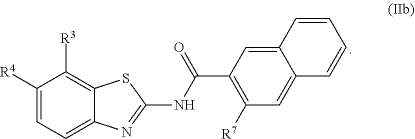

In some embodiments, provided herein is a compound having Formula (IIc):

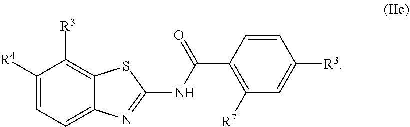

In some embodiments, provided herein is a compound having Formula (IId):

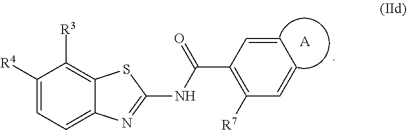

In some embodiments, provided herein is a compound having Formula (IIe):

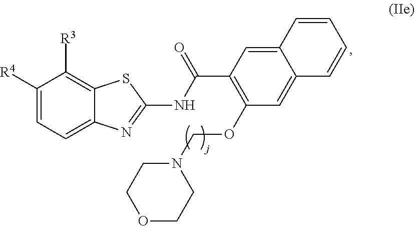

wherein j is 2, 3, 4, 5 or 6. In some embodiments, R⁴ is also C(O)NR^{c4}R^{d4}, S(O)₂R^{b4}, benzyl, NO₂, NR^{c4}R^{d4}, NR^{c4}C(O)R^{b4}, CN, or C(O)OR^{a4}S(O)₂R^{b4}. In some embodiments, R⁴ is S(O)₂R^{b4} and R^{b4} is a 4-10 membered heterocycloalkyl, such as pyrrolidinyl, morpholinyl, azetidinyl, or piperidinyl; wherein said, pyrrolidinyl, morpholinyl, azetidinyl, and piperidinyl are each optionally substituted with halo, C₁₋₄ alkyl, C₁₋₄ haloalkyl, OR^{a6}, SR^{a6}, or NR^{c6}R^{d6}. In some embodiments thereof, R³ is hydrogen, halo, C₁₋₄ haloalkyl, C₁₋₄ alkyl, NR^{c3}C(S)NR^{c3}R^{d3}

In some embodiments, provided herein is a compound having Formula (III):

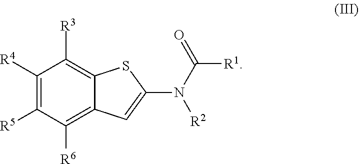

In some embodiments, provided herein is a compound having Formula (IIIa):

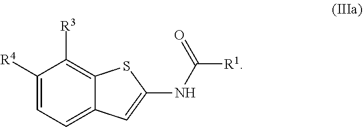

In some embodiments, provided herein is a compound having Formula IIIb:

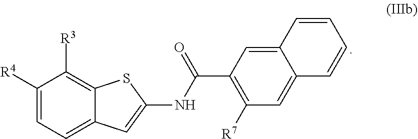

In some embodiments, provided herein is a compound having Formula (IIIc):

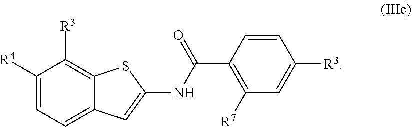

In some embodiments, provided herein is a compound having Formula (IIId):

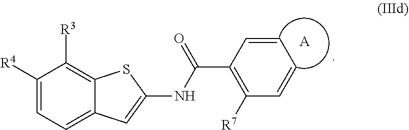

In some embodiments, provided herein is a compound having Formula (IIIe):

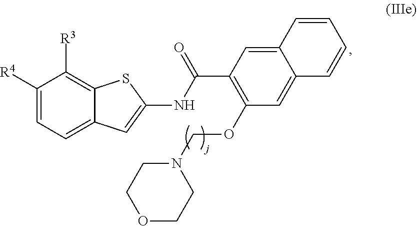

wherein j is 2, 3, 4, 5, or 6. In some embodiments, R⁴ is C(O)NR^{c4}R^{d4}, S(O)₂R^{b4}, benzyl, NO₂, NR^{c4}R^{d4}, NR^{c4}C(O)R^{b4}, CN, or C(O)OR^{a4}S(O)₂R^{b4}. In some embodiments, R⁴ is S(O)₂R^{b4}, and R^{b4} is a 4-10 membered heterocycloalkyl, such as pyrrolidinyl, morpholinyl, azetidinyl, or piperidinyl; wherein said, pyrrolidinyl, morpholinyl, azetidinyl, and piperidinyl are each optionally substituted with halo, C₁₋₄ alkyl, C₁₋₄ haloalkyl, OR^{a6}, SR^{a6}, or NR^{c6}R^{d6}. In some embodiments, R³ is hydrogen, halo, C₁₋₄ haloalkyl, C₁₋₄ alkyl, NR^{c3}C(S)NR^{c3}R^{d3}.

With respect to any formula(e) herein, X, R¹, R², R³, R⁴, R⁵, R⁶, R⁷, R⁸, R⁹, R¹⁰, R¹¹, R^{x}, Y¹, Y², R^{Y3}, Y⁴, R⁷, Y⁵, Y⁶, Y⁷, Y⁸, ring A, ring B, R^{Y1}, R^{Y2}, R^{Y4}, R^{Y5}, R^{Y6}, R^{Y7}, R^{Y8}, R^{Z1}, Z¹, Z², Z³, L¹, L², a, b, c, d, Q, Cy¹, Cy², Cy³, Cy⁴, Cy⁶, R^{a}, R^{b}, R^{c}, R^{d}, R^{e}, R^{a1}, R^{b1}, R^{c1}, R^{d1}, R^{e1}. R^{a2}, R^{b2}, R^{c2}, R^{d2}, R^{e2}, R^{a3}, R^{b3}, R^{c3}, R^{d3}, R^{e3}, R^{a4}, R^{b4}, R^{c4}, R^{d4}, R^{e4}, R^{a5}, R^{b5}, R^{c5}, R^{d5}, R^{e5}, R^{a6}, R^{b6}, R^{c6}, R^{d6} and R^{e6} are each as defined herein.

In some embodiments, the compound of Formula (I) is selected from:
3-[2-(morpholin-4-yl)ethoxy]-N-[6-(morpholine-4-sulfonyl)-1,3-benzothiazol-2-yl]naphthalene-2-carboxamide;
3-[4-(morpholin-4-yl)butoxy]-N-[6-(morpholine-4-sulfonyl)-1,3-benzothiazol-2-yl]naphthalene-2-carboxamide;
N-(6-benzyl-1,3-benzothiazol-2-yl)-3-[2-(morpholin-4-yl)ethoxy]naphthalene-2-carboxamide;
3-[2-(morpholin-4-yl)ethoxy]-N-(6-nitro-1,3-benzothiazol-2-yl)naphthalene-2-carboxamide;
N-(6-cyclohexaneamido-1,3-benzothiazol-2-yl)-3-[2-(morpholin-4-yl)ethoxy]naphthalene-2-carboxamide;
3-[4-(morpholin-4-yl)butoxy]-N-(6-nitro-1,3-benzothiazol-2-yl)naphthalene-2-carboxamide;
N-(6-cyano-1,3-benzothiazol-2-yl)-3-[2-(morpholin-4-yl)ethoxy]naphthalene-2-carboxamide;
N-(6-cyano-1,3-benzothiazol-2-yl)-3-[4-(morpholin-4-yl)butoxy]naphthalene-2-carboxamide;
3-[2-(morpholin-4-yl)ethoxy]-N-{7-[(phenylcarbamothioyl) amino]-1,3-benzothiazol-2-yl}naphthalene-2-carboxamide;
N-(6-methanesulfonamido-1,3-benzothiazol-2-yl)-3-[4-(morpholin-4-yl)butoxy]naphthalene-2-carboxamide;
3-[2-(morpholin-4-yl)ethoxy]-N-[6-(pyrrolidine-1-sulfonyl)-1,3-benzothiazol-2-yl]naphthalene-2-carboxamide;
N-[6-(dimethylsulfamoyl)-1,3-benzothiazol-2-yl]-3-[2-(morpholin-4-yl)ethoxy]naphthalene-2-carboxamide;
3-[4-(morpholin-4-yl)butoxy]-N-[6-(pyrrolidine-1-sulfonyl)-1,3-benzothiazol-2-yl]naphthalene-2-carboxamide;
3-[4-(morpholin-4-yl)butoxy]-N-(7-{[(pyridin-2-yl) carbamothioyl]amino}-1,3-benzothiazol-2-yl) naphthalene-2-carboxamide;
3-[2-(morpholin-4-yl)ethoxy]-N-(7-{[(pyridin-2-yl)carbamothioyl] amino}-1,3-benzothiazol-2-yl)naphthalene-2-carboxamide;
N-[6-(azetidine-1-sulfonyl)-1,3-benzothiazol-2-yl]-3-[2-(morpholin-4-yl)ethoxy]naphthalene-2-carboxamide;
N-[6-(azetidine-1-sulfonyl)-1,3-benzothiazol-2-yl]-3-[4-(morpholin-4-yl)butoxy]naphthalene-2-carboxamide;
3-[2-(morpholin-4-yl)ethoxy]-N-[6-(piperidine-1-sulfonyl)-1,3-benzothiazol-2-yl]naphthalene-2-carboxamide;
3-[4-(morpholin-4-yl)butoxy]-N-[6-(piperidine-1-sulfonyl)-1,3-benzothiazol-2-yl]naphthalene-2-carboxamide;
N-{6-[(difluoromethyl)sulfanyl]-1,3-benzothiazol-2-yl}-3-[2-(morpholin-4-yl)
ethoxy]naphthalene-2-carboxamide;
N-{6-[(difluoromethyl) sulfanyl]-1,3-benzothiazol-2-yl}-3-[4-(morpholin-4-yl) butoxy]naphthalene-2-carboxamide;
3-[2-(morpholin-4-yl) ethoxy]-N-[6-(pyrrolidine-1-sulfonyl)-1,3-benzothiazol-2-yl]-[1,1′-biphenyl]-4-carboxamide;
3-[4-(morpholin-4-yl)butoxy]-N-[6-(pyrrolidine-1-sulfonyl)-1,3-benzothiazol-2-yl]-[1,1′-biphenyl]-4-carboxamide;
6-[2-(morpholin-4-yl)ethoxy]-N-[6-(pyrrolidine-1-sulfonyl)-1,3-benzothiazol-2-yl]-1-benzothiophene-5-carboxamide;
6-[4-(morpholin-4-yl)butoxy]-N-[6-(pyrrolidine-1-sulfonyl)-1,3-benzothiazol-2-yl]-1-benzothiophene-5-carboxamide;
3-[2-(morpholin-4-yl)ethoxy]-N-[6-(2,2,2-trifluoroethoxy)-1,3-benzothiazol-2-yl]naphthalene-2-carboxamide;
3-[4-(morpholin-4-yl)butoxy]-N-(6-{2-oxa-6-azaspiro[3.3]heptane-6-sulfonyl}-1,3-benzothiazol-2-yl)naphthalene-2-carboxamide;
N-{6-[(3-hydroxypyrrolidin-1-yl)sulfonyl]-1,3-benzothiazol-2-yl}-3-[2-(morpholin-4-yl)ethoxy] naphthalene-2-carboxamide;
6-[4-(morpholin-4-yl)butoxy]-N-[6-(pyrrolidine-1-sulfonyl)-1,3-benzothiazol-2-yl]-2H-1,3-benzodioxole-5-carboxamide;
6-[2-(morpholin-4-yl)ethoxy]-N-[6-(pyrrolidine-1-sulfonyl)-1,3-benzothiazol-2-yl]-2H-1,3-benzodioxole-5-carboxamide;
methyl 2-{3-[4-(morpholin-4-yl)butoxy]naphthalene-2-amido}-1,3-benzothiazole-6-carboxylate;
1-methyl-5-[2-(morpholin-4-yl)ethoxy]-N-[6-(pyrrolidine-1-sulfonyl)-1,3-benzothiazol-2-yl]-1H-indole-6-carboxamide;
methyl 2-{3-[2-(morpholin-4-yl)ethoxy]naphthalene-2-amido}-1,3-benzothiazole-6-carboxylate;
N-(6-{[(3R)-3-hydroxypyrrolidin-1-yl]sulfonyl}-1,3-benzothiazol-2-yl)-3-[2-(morpholin-4-yl)ethoxy]naphthalene-2-carboxamide;
N-(6-{[(3R)-3-hydroxypyrrolidin-1-yl]sulfonyl}-1,3-benzothiazol-2-yl)-3-[4-(morpholin-4-yl)butoxy]naphthalene-2-carboxamide;
3-[4-(morpholin-4-yl)butoxy]-N-[6-(pyrrolidine-1-sulfonyl)-1-benzothiophen-2-yl]-[1,1′-biphenyl]-4-carboxamide;
3-[4-(morpholin-4-yl)butoxy]-N-[6-(pyrrolidine-1-sulfonyl)-1-benzothiophen-2-yl]naphthalene-2-carboxamide;
3-[2-(morpholin-4-yl)ethoxy]-N-[6-(pyrrolidine-1-sulfonyl)-1-benzothiophen-2-yl]-[1,1′-biphenyl]-4-carboxamide;
6-[4-(morpholin-4-yl)butoxy]-N-[6-(pyrrolidine-1-sulfonyl)-1-benzothiophen-2-yl]-1-benzothiophene-5-carboxamide;
6-[4-(morpholin-4-yl)butoxy]-N-[6-(pyrrolidine-1-sulfonyl)-1-benzothiophen-2-yl]-2H-1,3-benzodioxole-5-carboxamide;
3-[2-(morpholin-4-yl)ethoxy]-N-[6-(pyrrolidine-1-sulfonyl)-1-benzothiophen-2-yl] naphthalene-2-carboxamide;
3-[2-(morpholin-4-yl)ethoxy]-N-[6-(pyrrolidine-1-sulfonyl)-1-benzothiophen-2-yl]naphthalene-2-carboxamide; and
6-[2-(morpholin-4-yl)ethoxy]-N-[6-(pyrrolidine-1-sulfonyl)-1-benzothiophen-2-yl]-1-benzothiophene-5-carboxamide;
3-[2-(morpholin-4-yl)ethoxy]-N-[6-(pyrrolidine-1-sulfonyl)-1,3-benzothiazol-2-yl]-1-benzothiophene-2-carboxamide;
3-[2-(4,4-difluoropiperidin-1-yl)ethoxy]-N-[6-(pyrrolidine-1-sulfonyl)-1,3-benzothiazol-2-yl]naphthalene-2-carboxamide;
3-[4-(4,4-difluoropiperidin-1-yl)butoxy]-N-[6-(pyrrolidine-1-sulfonyl)-1,3-benzothiazol-2-yl]naphthalene-2-carboxamide;
N-(6-{[(3S)-3-hydroxypyrrolidin-1-yl]sulfonyl}-1,3-benzothiazol-2-yl)-3-[2-(morpholin-4-yl)ethoxy]naphthalene-2-carboxamide;
N-(6-{[(3S)-3-hydroxypyrrolidin-1-yl]sulfonyl}-1,3-benzothiazol-2-yl)-3-[4-(morpholin-4-yl)butoxy]naphthalene-2-carboxamide;

or a pharmaceutically acceptable salt thereof.

In some embodiments, provided herein is a compound selected from:
N-{6-[(difluoromethyl)sulfanyl]-1,3-benzothiazol-2-yl}naphthalene-2-carboxamide;
N-[6-(2,2,2-trifluoroethoxy)-1,3-benzothiazol-2-yl]naphthalene-2-carboxamide;
N-(6-methanesulfonamido-1,3-benzothiazol-2-yl)naphthalene-2-carboxamide;
N-(6-cyclohexaneamido-1,3-benzothiazol-2-yl)naphthalene-2-carboxamide;
N-{6-[(trifluoromethyl) sulfanyl]-1,3-benzothiazol-2-yl}naphthalene-2-carboxamide;
N-[6-(1H-1,3-benzodiazol-2-yl)-1,3-benzothiazol-2-yl]naphthalene-2-carboxamide;
N-[7-(trifluoromethyl)-1,3-benzothiazol-2-yl]naphthalene-2-carboxamide;
N-{6-[(cyclopropylcarbamoyl) methyl]-1,3-benzothiazol-2-yl}naphthalene-2-carboxamide;
N-(2,6-dichlorophenyl)-2-(naphthalene-2-amido)-1,3-benzothiazole-6-carboxamide;
2-(naphthalene-2-amido)-N-(pyridin-2-yl)-1,3-benzothiazole-6-carboxamide;
N-{6-[(2,6-dichlorophenyl)carbamoyl]-1,3-benzothiazol-2-yl}quinoline-6-carboxamide;
N-[6-(azetidine-1-sulfonyl)-1,3-benzothiazol-2-yl]-[1,1′-biphenyl]-4-carboxamide;
N-[6-(azetidine-1-sulfonyl)-1,3-benzothiazol-2-yl]-1-methyl-1H-indole-6-carboxamide;
N-[6-(azetidine-1-sulfonyl)-1,3-benzothiazol-2-yl]-1-methyl-1H-indole-2-carboxamide;
1-methyl-N-[6-(pyrrolidine-1-sulfonyl)-1,3-benzothiazol-2-yl]-1H-indole-2-carboxamide; and
4-(pyridin-3-yl)-N-[6-(pyrrolidine-1-sulfonyl)-1,3-benzothiazol-2-yl]benzamide;
   or a pharmaceutically acceptable salt thereof.

In some embodiments, provided herein is a compound selected from:
N-(6-acetamido-1,3-benzothiazol-2-yl) naphthalene-2-carboxamide;
N-(6-methanesulfonyl-1,3-benzothiazol-2-yl) naphthalene-2-carboxamide;
methyl 2-(naphthalene-2-amido)-1,3-benzothiazole-6-carboxylate;
N-[6-(dimethylsulfamoyl)-1,3-benzothiazol-2-yl]naphthalene-2-carboxamide;
N-(6-methyl-1,3-benzothiazol-2-yl) naphthalene-2-carboxamide;
N-[6-(morpholine-4-sulfonyl)-1,3-benzothiazol-2-yl]naphthalene-2-carboxamide;
N-[6-(piperidine-1-sulfonyl)-1,3-benzothiazol-2-yl]naphthalene-2-carboxamide;
N-(6-chloro-1,3-benzothiazol-2-yl)naphthalene-2-carboxamide;
N-(6-benzamido-1,3-benzothiazol-2-yl)naphthalene-2-carboxamide;
N-[2-(naphthalene-2-amido)-1,3-benzothiazol-6-yl]furan-2-carboxamide;
N-[6-(pyrrolidine-1-sulfonyl)-1,3-benzothiazol-2-yl]naphthalene-2-carboxamide;
   and
N-[6-(methylsulfamoyl)-1,3-benzothiazol-2-yl]naphthalene-2-carboxamide;

or a pharmaceutically acceptable salt thereof.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted. The substituents are independently selected, and substitution may be at any chemically accessible position. It is to be understood that substitution at a given atom is limited by valency. Throughout the definitions, the term “Ci-Cj” indicates a range which includes the endpoints, wherein i and j are integers and indicate the number of carbons. Examples include C₁-C₄, C₁-C₆, and the like.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1, 2, 3, 4-tetrahydro-naphthalene is an example of a 10-membered cycloalkyl group.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the term “Cᵢ-Cⱼ alkyl,” employed alone or in combination with other terms, refers to a saturated hydrocarbon group that may be linear, branched. In some embodiments, the alkyl group contains from 1 to 10, 1 to 6, 1 to 4, or from 1 to 3 carbon atoms. Examples of alkyl moieties include, but are not limited to, chemical groups such as methyl, ethyl, n-propyl, isopropyl, n-butyl, s-butyl, and t-butyl.

As used herein, the term “Cᵢ-Cⱼ alkoxy,” employed alone or in combination with other terms, refers to a group of formula —O-alkyl, wherein the alkyl group has i to j carbon atoms. Example alkoxy groups include, but are not limited to, methoxy, ethoxy, and propoxy (e.g., n-propoxy and isopropoxy). In some embodiments, the alkyl group has 1 to 3 carbon atoms or 1 to 4 carbon atoms.

As used herein, the term “Cᵢ-Cⱼ alkylamino” refers to a group of formula —NH(alkyl), wherein the alkyl group has i to j carbon atoms. In some embodiments, the alkyl group has 1 to 6, or 1 to 4 carbon atoms.

As used herein, the term “Cᵢ-Cⱼ dialkylamino” refers to a group of formula —N(alkyl)₂, wherein the two alkyl groups each has, independently, i to j carbon atoms. In some embodiments, each alkyl group independently has 1 to 6 or 1 to 4 carbon atoms.

As used herein, the term “thio” refers to a group of formula —SH.

As used herein, the term “Cᵢ-Cⱼ alkylthio” refers to a group of formula —S-alkyl, wherein the alkyl group has i to j carbon atoms. In some embodiments, the alkyl group has 1 to 6 or 1 to 4 carbon atoms.

As used herein, the term “amino” refers to a group of formula —NH₂.

As used herein, the term “halo”, used alone or in combination with other terms, refers to a halogen atom selected from F, Cl, I or Br. In some embodiments, “halo” refers to a halogen atom selected from F, Cl, or Br. In some embodiments, the halo group is F.

As used herein, the term “Cᵢ-Cⱼ haloalkyl,” employed alone or in combination with other terms, refers to an alkyl group having from one halogen atom to 2s+1 halogen atoms which may be the same or different, where “s” is the number of carbon atoms in the alkyl group, wherein the alkyl group has i to j carbon atoms. Examples of haloalkyl groups include, but are not limited to, fluoromethyl, difluoromethyl, or trifluoromethyl. In some embodiments, the haloalkyl group is trifluoromethyl. In some embodiments, the haloalkyl group has 1 to 6 or 1 to 4 carbon atoms.

As used herein, the term “Cᵢ-Cⱼ haloalkoxy,” employed alone or in combination with other terms, refers to a group of formula —O— Cᵢ-Cⱼ haloalkyl. Examples of haloalkoxy groups include, but are not limited to, fluoromethoxy, difluoromethoxy, or trifluoromethoxy. In some embodiments, the haloalkoxy group is trifluoromethoxy. In some embodiments, the haloalkoxy group has 1 to 6 or 1 to 4 carbon atoms.

As used herein the term “aryl”, when used alone or in combination with other terms, has the broadest meaning generally understood in the art, and can include an aromatic hydrocarbon ring or aromatic hydrocarbon ring system. An aryl group can be monocyclic, bicyclic or polycyclic, and may optionally include one to three additional ring structures; such as, for example, a cycloalkyl, a cycloalkenyl, a heterocycloalkyl, a heterocycloalkenyl, or a heteroaryl. The term “aryl” includes, without limitation, phenyl (benzenyl), naphthyl, tolyl, xylyl, anthracenyl, phenanthryl, azulenyl, biphenyl, naphthalenyl, 1-methylnaphthalenyl, acenaphthenyl, acenaphthylenyl, anthracenyl, fluorenyl, phenalenyl, phenanthrenyl, benzo[a]anthracenyl, benzo[c]phenanthrenyl, chrysenyl, fluoranthenyl, pyrenyl, tetracenyl (naphthacenyl), triphenylenyl, anthanthrenyl, benzopyrenyl, benzo[a]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl. In some embodiments, aryl is C₆₋₁₀ aryl. In some embodiments, the aryl group is a naphthalenyl ring or phenyl ring. In some embodiments, the aryl group is phenyl. In other embodiments, the aryl group is a naphthyl.

As used herein, the term “heteroaryl,” employed alone or in combination with other terms, refers to a monocyclic or polycyclic (e.g., having 2, 3 or 4 fused rings) aromatic hydrocarbon moiety in which at least one of the ring carbon atoms has been replaced with a heteroatom selected from nitrogen, sulfur and oxygen. Such a heteroaryl group may be attached through a ring carbon atom or, where valency permits, through a ring nitrogen atom. In some embodiments, the heteroaryl group is a 5- to 10-membered heteroaryl ring, which is monocyclic or bicyclic and which has 1, 2, 3, or 4 heteroatom ring members independently selected from nitrogen, sulfur and oxygen. In some embodiments, the heteroaryl group is a 5- to 6-membered heteroaryl ring, which is monocyclic and which has 1, 2, 3, or 4 heteroatom ring members independently selected from nitrogen, sulfur and oxygen. When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridine, pyrimidine, pyrazine, pyridazine, pyrrole, pyrazole, azolyl, oxazole, thiazole, imidazole, furan, thiophene, quinoline, isoquinoline, indole, benzothiophene, benzofuran, benzisoxazole, imidazo[1,2-b]thiazole, purine, benzodioxole, and the like.

A 5-membered heteroaryl is a heteroaryl group, as defined herein, having five ring-forming atoms comprising carbon and one or more (e.g., 1, 2, or 3) ring atoms independently selected from N, O, and S. Example five-membered heteroaryls include, but are not limited to, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A 6-membered heteroaryl is a heteroaryl group, as defined herein, having six ring-forming atoms wherein one or more (e.g., 1, 2, or 3) ring atoms are independently selected from N, O, and S. Example six-membered heteroaryls include, but are not limited to, pyridyl, pyrazinyl, pyrimidinyl, triazinyl and pyridazinyl.

The term “cycloalkyl”, as used herein, means a cyclic, monovalent hydrocarbon group of formula —CₙH₍₂ₙ₋₁₎ containing at least three carbon atoms, wherein n is an integer ranging from 3 to 10. The cycloalkyl group may be monocyclic or bicyclic In some embodiments, the cycloalkyl is a C₃₋₇ cycloalkyl. Non-limiting examples include cyclopropyl, cyclobutyl, cyclopentyl and cyclohexyl.cycloheptyl, norbornyl, and the like.

The term “heterocycloalkyl”, as used herein, refers to a cycloalkyl group in which one or more of the ring methylene groups (—CH₂—) has been replaced with a heteroatom selected from —O—, —S— or —NR—, in which R is H or C₁-C₆ alkyl or R is replaced by a bond through which the group is attached. The heterocycloalkyl group contains one and may contain up to four heteratoms. It excludes heteroaryl. In some embodiments, the heterocycloalkyl contains 4 to 7 ring atoms and in another embodiment, 5 or 6 ring atoms. In an embodiment, the heterocycloalkyl contains one or two heteroatoms. In another embodiment, the ring heteroatoms in the heterocycoalkyl is N and O. In some examples, the heterocycloalkyl contains one nitrogen ring atom and one oxygen ring atom, two nitrogen ring atom, one nitrogen ring atom or one or two oxygen ring atoms. Non-limiting examples include. pyrrolidinyl, imidazolidinyl, pyrazolidinyl, piperidyl, piperazinyl, morpholinyl, dioxanyl, and the like.

The term “alkenyl”, as used herein, means a straight or branched chain monovalent hydrocarbon group containing at least one carbon-carbon double bond and at least two carbon atoms. Non-limiting examples include ethenyl, propenyl, butenyl, 2-methylpropenyl, pentenyl and hexenyl.

The term “alkynyl”, as used herein, means a straight or branched chain monovalent hydrocarbon group containing at least one carbon-carbon triple bond and at least two carbon atoms. Non-limiting examples include ethynyl, propynyl, butynyl, pentynyl and hexynyl.

The compounds described herein can be asymmetric (e.g., having one or more stereocenters). All stereoisomers, such as enantiomers and diastereoisomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as d-camphorsulfonic acid or 1-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention can also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1, 2, 4-triazole, 1H- and 2H-isoindole, and 1H- and 2H-pyrazole.

Compounds of the invention can also include all isotopes of atoms occurring in the intermediates or final compounds. Isotopes include those atoms having the same atomic number but different mass numbers. For example, isotopes of hydrogen include tritium and deuterium.

The term “compound,” as used herein, is meant to include all stereoisomers, geometric isomers, tautomers, and isotopes of the structures depicted. Compounds herein identified by name or structure as one particular tautomeric form are intended to include other tautomeric forms unless otherwise specified. Compounds herein identified by name or structure without specifying the particular configuration of a stereocenter are meant to encompass all the possible configurations at the stereocenter. For example, if a particular stereocenter in a compound of the invention could be R or S, but the name or structure of the compound does not designate which it is, then the stereocenter can be either R or S.

All compounds, and pharmaceutically acceptable salts thereof, can be found together with other substances such as water and solvents (e.g., hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “RT” as used herein, are understood in the art, and refer generally to a temperature, e.g., a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (CH₃CN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19, and in Stahl et al., Handbook of Pharmaceutical Salts: Properties, Selection, and Use, (Wiley, 2002).

The below list is a key to abbreviations that may be used throughout.

Abbreviations

| Abbreviation | Definition |
|---|---|
| AcOH | Acetic acid |
| ALK5 | Aetivin Receptor-Like Kinase Receptor 5 |
| BTLA | B and T lymphocyte attenuator |
| (Boc)₂O | Di-tert-butyl dicaronate |
| CAS | Chemical Abstract Service registry number |
| CCR | Chemokine receptor type |
| CTLA4 | Cytotoxic T lymphocyte associated protein 4 |
| DIAD | Diisonrobyl azodicarboxylate |
| DCM | Dichloromethane |
| DIPEA | N,N-diisopropylethylamine |
| DMF | Dimethyl formamide |
| DMSO | Dirnethyl sulfoxide |
| DPPA | Diphenylphosphoryl azide |
| EtOAc | Ethyl acetate |
| FBS | Fetal bovine serum |
| Fe | Iron |
| H | Hour(s) |
| HA | hemagglutination assay |
| HATU | 1-[Bis(dimethylamino)methylene]-1H-1,2,3- |
| | triazolo[4,5-b]pyridinium 3-oxid hexafluorophosphate |
| Hex | Hexanes |
| KIR | Killer cell immunoglobulin-like receptor |
| LAG3 | Lymphocyte activation gene 3 |
| Min | Minute(s) |
| Ml | Milliliter(s) |
| HPLC | High-performance liquid chromatography |
| ICD | Immunogenic Cell Death |
| IFN | Interferon |
| IRF3 | Interferon regulatory transcription factor (IRF) family 3 |
| ISO | IFN-stimulated genes |
| LC/MS | Liquid chromatography/mass spectrometry |
| LiOH | Lithium hydroxide |
| MeOH | Methanol |
| MS | Mass spectrometry |
| NaH | Sodium hydride |
| NMP | N-Methyl-2-pyrrolidone |
| PDL | Programmed death ligand |
| PDGFR-2 | Plasminogen-related growth factor receptor 2 |
| PMA | Phorbol 12-myristate 13-acetate |
| RLR | RIG-I-like receptor |
| RPMI | Roswell park memorial institute medium |
| RT | Room Temperature |
| t-BuOH | Tert-Butanol |
| TBTU | O-(benzotriazol-1-yl)-N,N,N′,N′- |
| | tetramethyluronium tetrafluoroborate |
| TEA | Triethylamine |
| TFA | trifluoroacetic acid |
| THF | Tetrahydrofuran |
| TIM3 | T cell immunoglobulin and mucin domain 3 |
| TLR | Toll-like receptor |
| U | Units |
| uM | Micromolar |
| VISTA | V-domain Ig suppressor of T cell activation |

### Exemplary Synthesis

Exemplary procedures for making compounds described herein are provided below with reference to Scheme 1. Optimum reaction conditions and reaction times may vary depending on the particular reactants used. Unless otherwise specified, solvents, temperatures, pressures and other reaction conditions are readily selected by one of ordinary skill in the art. Specific procedures are provided in the Examples section. Compounds are named using the “structure to name” function included in MarvinSketch 5.9.0.

Typically, reaction progress may be monitored by thin layer chromatography (TLC) or HPLC-MS if desired. Intermediates and products may be purified by chromatography on silica gel, recrystallization, HPLC and/or reverse phase HPLC. In the reactions described below, it may be necessary to protect reactive functional groups (such as hydroxy, amino, thio, or carboxy groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed. (1999)). One or more deprotection steps in the synthetic schemes may be required to ultimately afford compounds of Formula I. The protecting groups depicted in the schemes are used as examples, and may be replaced by other compatible alternative groups. Starting materials used in the following schemes can be purchased or prepared by methods described in the chemical literature, or by adaptations thereof, using methods known by those skilled in the art. The order in which the steps are performed can vary depending on the protecting or functional groups introduced and the reagents and reaction conditions used, but would be apparent to those skilled in the art.

Compounds of the invention can be prepared as shown in the following schemes. The benzothiazole compounds can be prepared, for example, as shown in Scheme 1. To an appropriately nitro substituted aryl sulfonyl choride (A) is added a primary or secondary amine in excess to provide the corresponding aryl sulfonamide (B). Hydrogenation of the aromatic nitro group of compound (B) with Pd/C in a solvent (e.g., methanol) provides the corresponding aniline (C), which can then be converted to the benzothiazole by treatment with NH₄SCN in the presence of bromine (Br₂) in an acidic solvent (e.g., acetic acid) to provide benzothiazole (D).

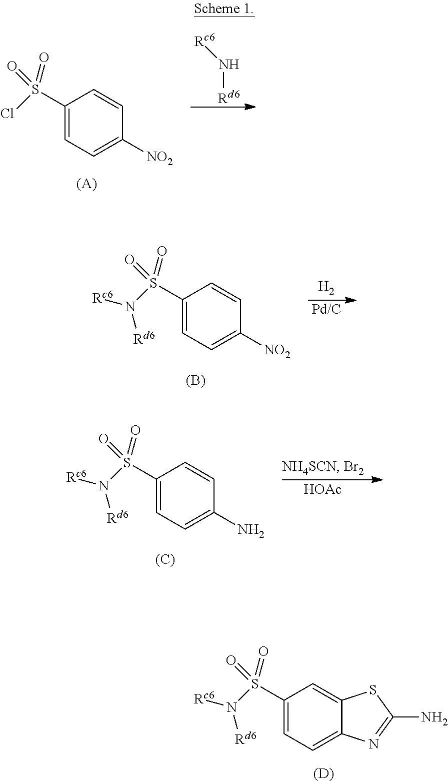

As shown in Scheme 2, compounds bearing additional substitution (R³ and R⁴) at the benzothiazole core can be synthesized in a similar fashion. For example, an appropriate substituted nitro aromatic compound (E) is reduced under hydrogenation conditions in the presence of Pd/C in a solvent, such as methanol, to provide aniline (F). Treatment with NH₄SCN in the presence of bromine (Br₂) in an acidic solvent (e.g., acetic acid) provides benzothiazole (G).

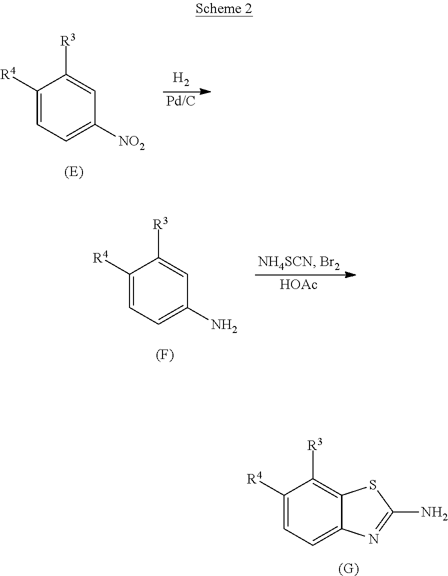

Benzothiophene compounds of the disclosure can be prepared according to Scheme 3. Amine (H) can be converted to the sulfonyl chloride (I) using SO₂ (gas) in acetic acid. Conversion to the sulfonamide (J) can be accomplished by reacting the sulfonyl chloride (I) with an excess of a primary or secondary amine. Reduction of the ester (J) with a reducing agent (e.g., LiAIH₄) in a solvent (e.g., THF) provides alcohol (K). Treatment of alcohol (K) with an oxidant (e.g., Dess-Martin reagent) in a solvent (e.g., DCM) provides aldehyde (L). The corresponding thiophene (M) is produced by reacting aldehyde (L) with methyl 2-mercaptoacetate in solvent (e.g., DMF) to provide benzo[b]thiophene-2-carboxylate (M). Ester hydrolysis with a base (e.g., LiOH) in a solvent (e.g., THF) provide the corresponding carboxylate (N). Curtius rearagement with DPPA in a solvent (e.g., triethylamine) in the presence of tert-butanol provides carbamate (O). Deprotection of the carbamate (O) with TFA provides amine (P).

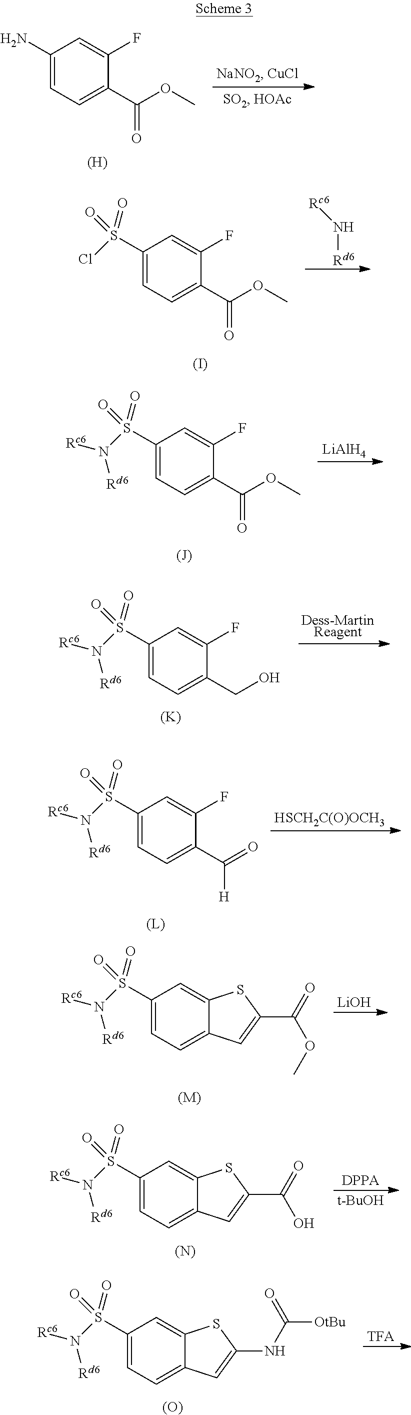

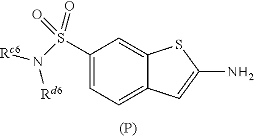

Substituted aromatic carboxylic acids can be produced according to Scheme 4. An appropriately substituted hydroxy substituted carboxylic acid (Q) is treated with an amino halide (X=Cl or Br) in a solvent (e.g., DMF) in the presence of a base (e.g., Cs₂CO₃) to provide the ether product (S).

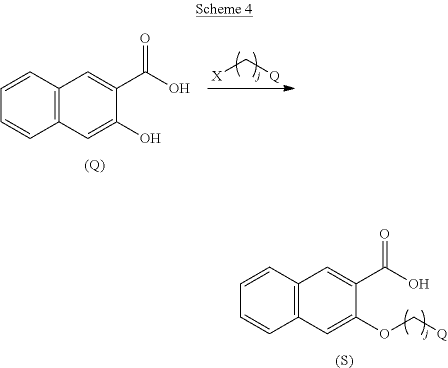

Amide products can be synthesized as shown in Scheme 5. Amine (G) can be coupled with a carboxylic acid (S), using standard peptide coupling reagents (e.g. HATU, DIPEA) in a solvent (e.g., DMF) to provide amide (U).

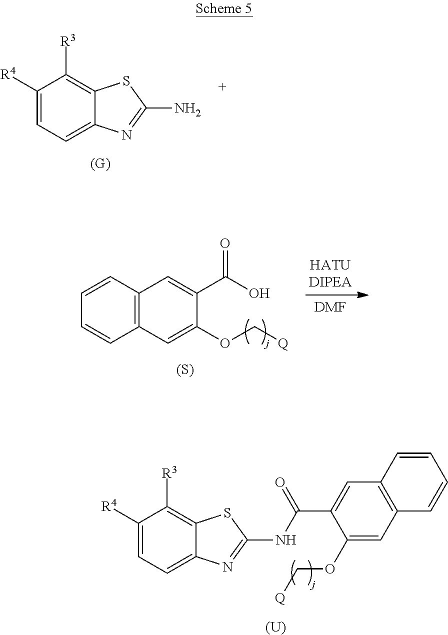

### Methods

The present disclosure provides methods of agonizing the retinoic acid-inducible gene-I pathway by contacting RIG-I with a compound of the invention, or a pharmaceutically acceptable salt thereof. In some embodiments, the invention provides methods for inducing the expression of cytokines or chemokines associated with the RIG-I pathway. Cytokines or chemokinates that are associated with the RIG-I pathway can include, for example, interferon sensitive response element (ISRE), proinflammatory cytokines, RANTES, and CXCL10.

The present disclosure further provides methods for activating interferon regulatory factor 3 (IRF3) by contacting IRF3 with a compound of the invention, or a pharmaceutically acceptable salt thereof. In some embodiments, the expression of IRF3-dependent genes is induced by a factor of about 1 to about 40-fold. In some embodiments, the expression of IRF3-dependent genes is induced by a factor in the range of 10-20-fold, 20-40-fold and greater than 40-fold.

In some embodiments, the expression of CXCL-10 (IP-10) is induced by a factor of about 10 to greater than about 1,600 pg/mL. In some embodiments, the expression of CXCL-10 (IP-10) is induced by a factor of 400-800 pg/mL, 800-1,600 pg/mL and greater than 1,600 pg/mL. In some embodiments, the induction of expression of IRF3 occurs within about 24 h following administration of a compound described herein or a pharmaceutically acceptable salt thereof. In some embodiments, the compounds described herein induce the expression of CXCL10 in cancer cells. In some embodiments, the cancer cells are colon carcinoma cells. In some embodiments, the compounds described herein stimulate the release of DAMPs.

In some embodiments, the contacting can be administering to a patient a compound provided herein, or a pharmaceutically acceptable salt thereof. In certain embodiments, the compounds of the present disclosure, or pharmaceutically acceptable salts thereof, are useful for therapeutic administration to enhance, stimulate and/or increase immunity in cancer. For example, a method of treating a disease or disorder can include administering to a patient in need thereof a therapeutically effective amount of a compound provided herein, or a pharmaceutically acceptable salt thereof. The compounds of the present disclosure can be used alone, in combination with other agents or therapies or as an adjuvant or neoadjuvant for the treatment of diseases or disorders, including cancers. For the uses described herein, any of the compounds of the disclosure, including any of the embodiments thereof, may be used.

Diseases and disorders that are treatable using compounds of the present disclosure include, but are not limited to, cell-proliferation disorders and immune-related diseases. In some embodiments, the cell-proliferation disorder is cancer, benign papillomatosis, a gestational trophoblastic disease, or a benign neoplastic disease (e.g., skin papilloma [warts] and genital papilloma). In some embodiments, the cell-proliferation disorder is a cancer.

Examples of cancers that are treatable using compounds of the present disclosure include, but are not limited to, brain cancer, cancer of the spine, cancer of the head, cancer of the neck, leukemia, blood cancers, cancer of the reproductive system, gastrointestinal cancer, liver cancer, bile duct cancer, kidney cancer, bladder cancer, bone cancer, lung cancer, malignant mesothelioma, sarcomas, lymphomas, glandular cancer, thyroid cancer, heart cancer, malignant neuroendocrine (carcinoid) tumors, midline tract cancers, and metastazied cancers.

In specific embodiments, cancers of the brain and spine include anaplastic astrocytomas, glioblastomas, astrocytomas, and estheosioneuroblastomas (also known as olfactory blastomas). In particular embodiments, the brain cancer includes astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, cancers of the head and neck include nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers ofthe larynx (e.g., laryngeal squamous cell carcinomas, rhabdomyosarcomas), and cancers of the eye or ocular cancers (e.g., intraocular melanoma and retinoblastoma).

In specific embodiments, leukemia and cancers of the blood include myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blastphase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic

In specific embodiments, skin cancers include melanoma, squamous cell cancers, and basal cell cancers.

In specific embodiments, reproductive system cancers include breast cancers, cervical cancers, vaginal cancers, ovarian cancers, prostate cancers, penile cancers, and testicular cancers. In specific instances of these embodiments, breast cancer includes ductal carcinomas and phyllodes tumors. In specific instances of these embodiments, the breast cancer may be male breast cancer or female breast cancer. In specific instances of these embodiments, cervical cancer includes squamous cell carcinomas and adenocarcinomas. In specific instances of these embodiments, the cancer is an ovarian cancer selected from the group consisting of epithelial cancers.

In specific embodiments, gastrointestinal cancers include esophageal cancers, gastric cancers (also known as stomach cancers), gastrointestinal carcinoid tumors, pancreatic cancers, gallbladder cancers, colorectal cancers, and anal cancer, and can include esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the liver cancer is hepatocellular carcinoma.

In particular embodiments, the cancer is bile duct cancer (also known as cholangiocarcinoma) including intrahepatic cholangiocarcinoma and extrahepatic cholangiocarcinoma.

In specific embodiments, kidney and bladder cancers include renal cell cancer, Wilms tumors, and transitional cell cancers. In particular embodiments, the cancer is a bladder cancer, including urethelial carcinoma (a transitional cell carcinoma), squamous cell carcinomas, and adenocarcinomas.

In specific embodiments, bone cancers include osteosarcoma, malignant fibrous histiocytoma of bone, Ewing sarcoma, and chordoma (cancer of the bone along the spine).

In specific embodiments, lung cancers include non-small cell lung cancer, small cell lung cancers, bronchial tumors, and pleuropulmonary blastomas.

In specific embodiments, the cancer is selected from malignant mesothelioma, consisting of epithelial mesothelioma and sarcomatoids.

In specific embodiments, sarcomas include central chondrosarcoma, central and periosteal chondroma, fibrosarcoma, clear cell sarcoma of tendon sheaths, and Kaposi's sarcoma.

In specific embodiments, lymphoma cancers include Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, glandular cancers include adrenocortical cancer (also known as adrenocortical carcinoma or adrenal cortical carcinoma), pheochromocytomas, paragangliomas, pituitary tumors, thymoma, and thymic carcinomas.

In specific embodiments, thyroid cancers include medullary thyroid carcinomas, papillary thyroid carcinomas, and follicular thyroid carcinomas.

In specific embodiments, the cancer is selected from germ cell tumors, include malignant extracranial germ cell tumors and malignant extragonadal germ cell tumors. In specific instances of these embodiments, the malignant extragonadal germ cell tumors include nonseminomas and seminomas.

In specific embodiments, heart tumor cancers include malignant teratoma, lymphoma, rhabdomyosacroma, angiosarcoma, chondrosarcoma, infantile fibrosarcoma, and synovial sarcoma.

In certain other embodiments, the methods include, but are not limited to, administering a compound described herein to a subject in order to induce immunogenic cell death of cancer cells (e.g., tumor cells). In other embodiments, the methods include but are not limited to administering the compound to induce T cell responses including memory T cell responses specific to cancer antigens.

In further aspects, the invention provides methods for inducing an innate immune response in a subject, comprising administering a therapeutically effective amount of a compound described herein or a pharmaceutically acceptable salt thereof. In certain embodiments, the subject is a human.

The present disclosure also includes the following embodiments:

a compound of Formula I, or a pharmaceutically acceptable salt thereof, as defined in any of the embodiments described herein, for use as a medicament;

a compound of Formula I, or a pharmaceutically acceptable salt thereof, as defined in any of the embodiments described herein, for use in the treatment of the here above-mentioned indication; and

a compound of Formula I, or a pharmaceutically acceptable salt thereof, as defined in any of the embodiments described herein, for use in the treatment of a cell proliferation disorder, such as cancer;

the use of a compound of Formula I, or a pharmaceutically acceptable salt thereof, as defined in any of the embodiments described herein, for the manufacture of a medicament for treating a disease or condition for which an activator of the RIG-I pathway is indicated;

a compound of Formula I, or a pharmaceutically acceptable salt thereof, as defined in any of the embodiments described herein, for use in the treatment of a disease or condition for which an activator of the RIG-I pathway is indicated; and

a pharmaceutical composition for the treatment of a disease or condition for which an activator of the RIG-I pathway is indicated, comprising a compound of Formula I, or a pharmaceutically acceptable salt thereof, as defined in any of the embodiments described herein.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “prophylactic” refers to preventing the disease, i.e. causing the clinical symptoms or signs of the disease not to develop in a subject, such as a mammal that may be exposed to or predisposed to the disease but does not yet experience or display symptoms/signs of the disease;

### Combination Therapy

The compounds of the present disclosure can be administered with one or more agents. In certain embodiments, the one or more agents include an immune stimulator, including but not limited to a stimulator of T cells or dendritic cells. The one or more agents can be selected from, inter alia, the group consisting of adjuvants, CTLA-4 and PD-I pathway antagonists and other immunomodulatory agents, lipids, liposomes, peptides, anti-cancer and chemotherapeutic agents.

The CLTA-4 and PD-I pathways are important negative regulators of immune response. Activated T-cells up-regulate CTLA-4, which binds on antigen-presenting cells and inhibits T-cell stimulation, IL-2 gene expression, and T-cell proliferation; these anti-tumor effects have been observed in mouse models of colon carcinoma, metastatic prostate cancer, and metastatic melanoma. PD-I binds to active T-cells and suppresses T-cell activation; PD-I antagonists have demonstrated anti-tumor effects as well. CTLA-4 and PD-I pathway antagonists that may be used in combination with the compounds described herein, or the pharmaceutically acceptable salts thereof, include ipilimumab, tremelimumab, nivolumab, pembrolizumab, CT-011, AMP-224, and MDX1106.

“PD-1 antagonist” or “PD-1 pathway antagonist” refers to any chemical compound or biological molecule that blocks binding of PD-L1 expressed on a cancer cell to PD-I expressed on an immune cell (T-cell, B-cell, or NKT-cell), blocks binding of PD-L2 expressed on a cancer cell to the immune-cell expressed PD-L. Synonyms for PD-L include PD-I: PDCDI, PD1, CD279, and SLEB2 for PD-1; PDCD1L1, PDLI, B7H1, B7-4, CD274, and B7-H for PD-L1; and PDCD1L2, PDL2, B7-DC, Btdc, and CD273 for PD-L2.

Additionally, the use of cytotoxic agents may be used in combination with the compounds described herein, or pharmaceutically acceptable salts thereof, include, but are not limited to, arsenic trioxide (Trisenox®), asparaginase (also known as L-asparaginase, and Erwinia L-asparaginase, Elspar® and Kidrolase®).

Chemotherapeutic agents that may be used in combination with the compounds described herein, or pharmaceutically acceptable salts thereof, include abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-Lprolyl-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea and taxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine.

Examples of vascular endothelial growth factor (VEGF) receptor inhibitors that may be used with the compounds described herein include, but are not limited to, bevacizumab (AVASTIN by Genentech/Roche), axitinib, Brivanib Alaninate (BMS-582664), motesanib (SO 230), and sorafenib (NEXAVAR). Such inhibitors may be provided as a pharmaceutically acceptable salt, where appropriate.

Examples of topoisomerase II inhibitors that may be used with the compounds described herein include, but are not limited to, etoposide (also known as VP-16 and Etoposide phosphate, TOPOSAR, VEPESID, and ETOPOPFiOS), and teniposide (VUMON). Such inhibitors may be provided as a pharmaceutically acceptable salt, where appropriate.

Examples of alkylating agents that may be used with the compounds described herein include, but are not limited to, 5-azacytidine (VIDAZA), decitabine (DECOGEN), temozolomide (TEMODAR and TEMODAL), dactinomycin (COSMEGEN), melphalan (ALKERAN), altretamine (FiEXALEN), carmustine (BCNU), bendamustine (TREANDA), busulfan (Busuefex® and Myleran®), carboplatin (Paraplatin®), lomustine (CeeNU®), cisplatin (Platinol® and Platinol®-AQ), chlorambucil (Leukeran®), cyclophosphamide (Cytoxan® and Neosar®), dacarbazine (DTICDome), altretamine (FIexalen®), ifosfamide (Ifex®), procarbazine (Matulane®), mechlorethamine (Mustargen®), streptozocin (Zanosar®), thiotepa (Thioplex®). Such alkylating agents may be provided as a pharmaceutically acceptable salt, where appropriate.

Examples of anti-tumor antibiotics that may be used with the compounds described herein include, but are not limited to, doxorubicin (Adriamycin® and Rubex®), bleomycin (Lenoxane®), daunorubicin (Cerubidine®), daunorubicin liposomal (DaunoXome®), mitoxantrone (Novantrone®), epirubicin (Ellence™), idarubicin (Idamycin®, Idamycin PFS®), and mitomycin C (Mutamycin®). Such anti-tumor antibiotics may be provided as a pharmaceutically acceptable salt, where appropriate.

Examples of anti-metabolites that may be used with the compounds described herein include, but are not limited to, claribine (Leustatin®), 5-fluorouracil (Adrucil®, 6-thioguanine (Purinethol®), pemetrexed (Alimta®), cytarabine (Cytosar-U®), cytarabine liposomal (DepoCyt®), decitabine (Dacogen®), hydroxyurea and (Flydrea®, Droxia™ and Mylocel™) fludarabine (Fludara®), floxuridine (FUDR®), cladribine Leustatin™), methotrexate (Rheumatrex® and Trexall™), and pentostatin (Nipent®). Such anti-metabolites may be provided as a pharmaceutically acceptable salt, where appropriate.

Examples of retinoids that may be used with the compounds described herein include, but are not limited to, alitretinoin (Panretin®), tretinoin (Vesanoid®), Isotretinoin (Accutane®), and bexarotene (Targretin®). Such compounds may be provided as a pharmaceutically acceptable salt, where appropriate.

Immuno-oncology therapy agents (e.g., a checkpoint inhibitor) may also be used in combination with the compounds described herein. Representative immuno-oncology therapy agents include, for example, those targeting the adenosine A2A receptor, Activin Receptor-Like Kinase Receptor 5 (ALK5), BRAF, B7-H3, B7-H4, B and T lymphocyte attenuator (BTLA), cytotoxic T lymphocyte associated protein 4 (CTLA4), CSF1, CXCR2, CXCR4, chemokine receptor type 2 (CCR2), chemokine receptor type 5 (CCR5), indoleamine 2,3-dioxygenase (IDO), killer cell immunoglobulin-like receptor (KIR), lymphocyte activation gene 3 (LAG3), PDE5, plasminogen-related growth factor receptor 2 (PRGFR-2), T cell immunoglobulin and mucin domain 3 (TIM3), or V-domain Ig suppressor of T cell activation (VISTA).

Antigens and adjuvants that may be used in combination with the compounds described herein include B7 costimulatory molecule, interleukin-2, interferon-y, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly TC, used separately or in combination with the described compositions are also potential adjuvants. Such antigens and anjuvants may be provided as a pharmaceutically acceptable salt, where appropriate.

### Administration, Pharmaceutical Formulations, Dosage Forms

The compounds of the invention can be administered to patients (e.g., animals and humans) in need of such treatment in appropriate dosages that will provide prophylactic and/or therapeutic efficacy. The dose required for use in the treatment or prevention of any particular disease or disorder will typically vary from patient to patient depending on, for example, particular compound or composition selected, the route of administration, the nature of the condition being treated, the age and condition of the patient, concurrent medication or special diets then being followed by the patient, and other factors. The appropriate dosage can be determined by the treating physician.

A compound of this invention can be administered orally, subcutaneously, topically, parenterally, intratumorally or by inhalation spray or rectally in dosage unit formulations containing conventional non-toxic pharmaceutically acceptable carriers, adjuvants and vehicles. Parenteral administration can involve subcutaneous injections, intravenous or intramuscular injections or infusion techniques.

Treatment duration can be as long as deemed necessary by a treating physician. The compositions can be administered one to four or more times per day. A treatment period can terminate when a desired result, for example a particular therapeutic effect, is achieved. Or a treatment period can be continued indefinitely.

Pharmaceutical compositions that include the compounds of the invention are also provided. For example, the present invention provides a pharmaceutical composition comprising a compound of the invention, or a pharmaceutically acceptable salt thereof, and at least one pharmaceutically acceptable carrier.

In some embodiments, the pharmaceutical compositions can be prepared as solid dosage forms for oral administration (e.g., capsules, tablets, pills, dragees, powders, granules and the like). A tablet can be prepared by compression or molding. Compressed tablets can include one or more binders, lubricants, glidants, inert diluents, preservatives, disintegrants, or dispersing agents. Tablets and other solid dosage forms, such as capsules, pills and granules, can include coatings, such as enteric coatings.

Compositions for inhalation or insufflation include solutions and suspensions in pharmaceutically acceptable aqueous or organic solvents, or mixtures thereof, and powders. Liquid dosage forms for oral administration can include, for example, pharmaceutically acceptable emulsions, microemulsions, solutions, suspensions, syrups and elixirs. Suspensions can include one or more suspending agents

Dosage forms for transdermal administration of a subject composition include powders, sprays, ointments, pastes, creams, lotions, gels, solutions, patches and inhalants.

Compositions and compounds of the present invention can be administered by aerosol which can be administered, for example, by a sonic nebulizer.

Pharmaceutical compositions of this invention suitable for parenteral administration include a compound of the invention together with one or more pharmaceutically acceptable sterile isotonic aqueous or non-aqueous solutions, dispersions, suspensions or emulsions. Alternatively, the composition can be in the form of a sterile powder which can be reconstituted into a sterile injectable solutions or dispersion just prior to use.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

¹H NMR Spectra were acquired on one or more of three instruments: (1) Agilent UnityInova 400 MHz spectrometer equipped with a 5 mm Automation Triple Broadband (ATB) probe (the ATB probe was simultaneously tuned to ¹H, ¹⁹F and ¹³C); (2) Agilent UnityInova 400 MHz spectrometer; or (3) Varian Mercury Plus 400 MHz spectrometer. Several NMR probes were used with the 400 MHz NMR spectrometer, including both 3 mm and 5 mm ¹H, ¹⁹F and ¹³C probes and a 3 mm X¹H¹⁹F NMR probe (usually X is tuned to ¹³C). For typical ¹H NMR spectra, the pulse angle was 45 degrees, 8 scans were summed and the spectral width was 16 ppm (−2 ppm to 14 ppm). Typically, a total of about 32768 complex points were collected during the 5.1 second acquisition time, and the recycle delay was set to 1 second. Spectra were collected at 25° C. ¹H NMR Spectra were typically processed with 0.3 Hz line broadening and zero-filling to about 131072 points prior to Fourier transformation. Chemical shifts were expressed in ppm relative to tetramethylsilane. The following abbreviations are used herein: br=broad signal, s=singlet, d=doublet, dd=double doublet, ddd=double double doublet, dt=double triplet, t=triplet, td=triple doublet, tt=triple triplet q=quartet, m=multiplet.

Liquid chromatography—mass spectrometry (LCMS) experiments to determine retention times and associated mass ions were performed using one or more of the following Methods A, B, and C:An API 150EX mass spectrometer linked to a Shimadzu LC-10AT LC system with a diode array detector was used. The spectrometer had an electrospray source operating in positive and negative ion mode. LC was carried out using an Agilent ZORBAX XDB 50×2.1 mm C18 column and a 0.5 mL/minute flow rate. Solvent A: 95% water, 5% acetonitrile containing 0.01% formic acid; Solvent B: acetonitrile. The gradient was shown as below. 0-0.5 min: 2% solvent (B); 0.5-2.5 min: 2% solvent B to 95% solvent (B); 2.5-4.0 min: 95% solvent (B); 4.0-4.2 min: 95% solvent (B) to 2% solvent B; 4.2-6.0 min: 2% solvent (B). Compounds which required column chromatography were purified manually or fully automatically using either a Biotage SP1™ Flash Purification system with Touch Logic Control™ or a Combiflash Companion® with pre-packed silica gel Isolute® SPE cartridge, Biotage SNAP cartridge or Redisep® Rf cartridge respectively.

### Exemplary Preparation of Benzothiophene Intermediates

The following amines shown in Table 1 were used in preparing the compounds of the invention. They are either commercially available or can be prepared by known synthetic procedures. CAS registry numbers are provided for each intermediate.

TABLE 1
Commercial benzothiazoles.

| Intermediate | | | |
|---|---|---|---|
| No. | Structure | Name | CAS No. |
| 1 | 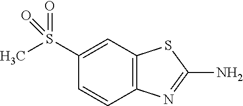 | 6-(methylsulfonyl)-2- benzothiazolamine | 17557-67-4 |
| 2 | 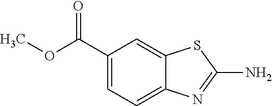 | 6-Carbomethoxy-2- aminobenzothiazole | 66947-92-0 |
| 3 | 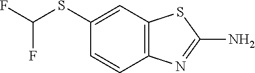 [SMILES: Nc1nc2ccc(SC(F)F)cc2s1] | 6-[(difluoromethyl)thio]- 2-benzothiazolamine | 325731-49-5 |
| 4 | 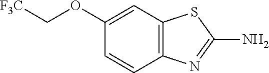 [SMILES: Nc1nc2ccc(OCC(F)(F)F)cc2s1] | 6-(2,2,2-trifluoroethoxy)- 2-benzothiazolamine | 131395-08-9 |
| 5 | 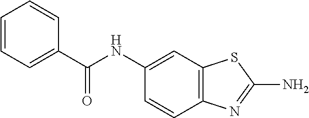 [SMILES: Nc1nc2ccc(NC(=O)c3ccccc3)cc2s1] | N-(2-amino-6- benzothiazolyl)- benzamide | 52603-58-4 |
| 6 | 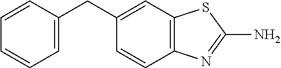 [SMILES: Nc1nc2ccc(Cc3ccccc3)cc2s1] | 6-(phenylmethyl)-2- benzothiazolamine | 129121-46-6 |
| 7 | 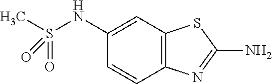 | N-(2-amino-6- benzothiazolyl)- methanesulfonamide | 108792-21-8 |
| 8 | 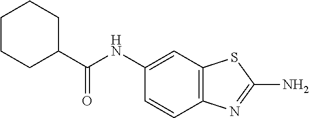 [SMILES: Nc1nc2ccc(NC(=O)C3CCCCC3)cc2s1] | N-(2-amino-6- benzothiazolyl)- cyclohexanecarboxamide | 351437-66-6 |
| 9 | 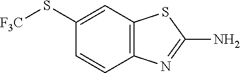 [SMILES: Nc1nc2ccc(SC(F)(F)F)cc2s1] | 6-[(trifluoromethyl)thio]- 2-benzothiazolamine | 326-45-4 |
| 10 | 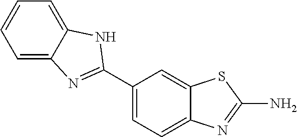 [SMILES: Nc1nc2ccc(-c3nc4ccccc4[nH]3)cc2s1] | 6-(1H-benzimidazol-2-yl)- 2-benzothiazolamine, | 314033-42-6 |
| 11 | 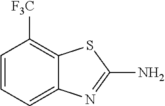 [SMILES: Nc1nc2cccc(C(F)(F)F)c2s1] | 7-(trifluoromethyl)-2- benzothiazolamine | 60388-39-8 |
| 12 | 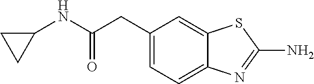 [SMILES: Nc1nc2ccc(CC(=O)NC3CC3)cc2s1] | 2-amino-N-cyclopropyl- 6-benzothiazoleacetamide | 1225700-12-8 |
| 13 | 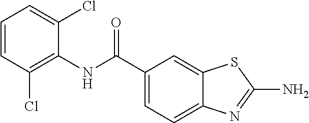 [SMILES: Nc1nc2ccc(C(=O)Nc3c(Cl)cccc3Cl)cc2s1] | 2-amino-N-(2,6- dichlorophenyl)-6- benzothiazolecarboxamide | 769961-89-9 |
| 14 | 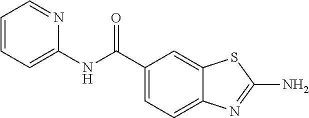 [SMILES: Nc1nc2ccc(C(=O)Nc3ccccn3)cc2s1] | 2-amino-N-2-pyridinyl-6- benzothiazolecarboxamide | 1225698-71-4 |
| 15 | 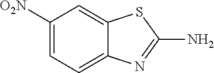 [SMILES: Nc1nc2ccc([N+](=O)[O-])cc2s1] | 6-nitro-2- benzothiazolamine | 6285-57-0 |
| 16 | 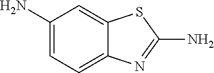 [SMILES: Nc1ccc2nc(N)sc2c1] | 2,6-benzothiazolediamine | 5407-51-2 |
| 17 | 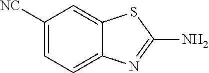 [SMILES: N#Cc1ccc2nc(N)sc2c1] | 2-amino-6- benzothiazolecarbonitrile | 19759-66-1 |
| 18 | 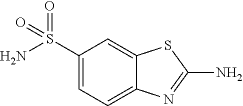 [SMILES: Nc1nc2ccc(S(N)(=O)=O)cc2s1] | 2-Amino-6- benzothiazolesulfonamide | 18101-58-1 |
| 18′ | 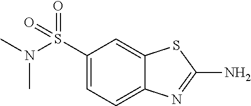 [SMILES: CN(C)S(=O)(=O)c1ccc2nc(N)sc2c1] | 2-amino-N,N-dimethyl- 1,3-benzothiazole-6- sulfonamide | 17901-13-2 |

### Intermediate 19: 3-(2-amino-1,3-benzothiazol-7-yl)-1-phenylthiourea

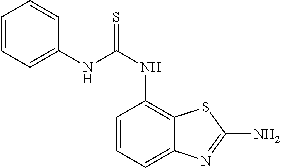 [SMILES: Nc1nc2cccc(NC(=S)Nc3ccccc3)c2s1]

Step 1: To a suspension of 2-amino-6-nitrobenzothiazole (CAS No. 6285-57-0, 1.8 g, 9.22 mmol) in ethanol (20 mL) and acetic acid (5 mL) was added iron powder (3.1 g, 55.32 mmol). The resulting mixture was refluxed under N₂ for 3 h. After cooling to room temperature, the reaction mixture was filtered through Celite and the filtrate was basified with 4 N aq. NaOH solution and extracted with ethyl acetate. The combined organic phases were washed with brine, dried over Na₂SO₄, and concentrated. The residue was purified through column chromatography (dichloromethane/methanol=30/1) to afford benzo[d]thiazole-2,7-diamine as a white solid (1.0 g, 66%). LC/MS (ES⁺) calcd for C₇H₇N₃S: 165.0; found: 166.0 [M+H]. ¹H NMR (400 MHz, DMSO-d6): δ 7.24 (s, 2H), 6.90 (t, J=7.8 Hz, 1H), 6.62 (d, J=8.0 Hz, 1H), 6.30 (d, J=8.0 Hz, 1H), 5.11 (s, 211).

Step 2: To a solution of benzo[d]thiazole-2,7-diamine (110 mg, 0.56 mmol) in NMP (5 mL) was added isothiocyanatobenzene (CAS No. 103-72-0, 114 mg, 0.85 mmol). The resulting mixture was stirred at 50° C. under N₂ for 3 h. The reaction was quenched with water, and extracted with ethyl acetate. The organic phases were washed with water, dried over Na₂SO₄, and concentrated under reduced pressure. The residue was purified through column chromatography (dichloromethane/methanol=30/1) to afford 1-(2-aminobenzo[d]thiazol-7-yl)-3-phenylthiourea as a white solid (110 mg, 65%). LC/MS (ES⁺) calcd for C₁₄H¹²N₄S₂: 300.1; found: 301.1 [M+H]. ¹H NMR (400 MHz, DMSO-d6): δ 9.84 (s, 1H), 9.72 (s, 1H), 7.50-7.44 (m, 4H), 7.32 (t, J=7.8 Hz, 2H), 7.21-7.18 (m, 2H), 7.12 (t, J=7.4 Hz, 1H), 6.99-6.94 (m, 1H).

### Intermediate 20: 3-(2-amino-1,3-benzothiazol-6-yl)-1-phenylthiourea

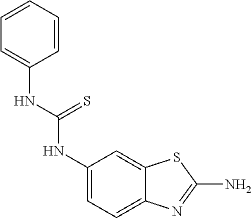 [SMILES: Nc1nc2ccc(NC(=S)Nc3ccccc3)cc2s1]

This compound can be prepared as described for 3-(2-amino-1,3-benzothiazol-7-yl)-1-phenylthiourea (Intermediate 19) substituting 2-amino-5-nitrobenzothiazole (CAS No. 1072-98-6) for 2-amino-6-nitrobenzothiazole in step 1. LC/MS (ES⁺) calcd for C₁₄H₁₂N₄S₂: 300.4; found: 301.4 [M+H]. ¹H NMR (400 MHz, DMSO-d6): δ 8.63 (s, 1H), 8.57 (s, 1H), 8.33-8.29 (m, 1H), 7.65-7.58 (m, 2H), 7.47-7.41 (m, 2H), 7.32-7.24 (m, 2H), 7.20 (d, J=6.96 Hz, 1H), 6.99-6.91 (m, 2H).

### Intermediate 21: 6-(azetidin-1-ylsulfonyl)benzo[d]thiazol-2-amine

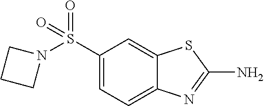 [SMILES: Nc1nc2ccc(S(=O)(=O)N3CCC3)cc2s1]

Step 1: To a solution of 4-nitrobenzene-1-sulfonyl chloride (CAS No. 98-74-8, 9.7 g, 43.8 mmol) in THF (30 mL) was added dropwise to a solution of azetidine (5 g, 87 mmol) in water (50 mL) at 0° C. The resulting suspension was stirred at room temperature for 1 h. The precipitates were filtered and washed with water, dried to afford 1-((4-nitrophenyl) sulfonyl)azetidine (8.8 g, 84%) as a light yellow solid. LCMS (ES⁺): m/z calculated for C₉H₁₀N₂O₄S: 242.0; found: 243.0 [M+H]. ¹H NMR (400 MHz, CDCl₃) δ 8.43 (d, J=8.8 Hz, 2H), 8.04 (d, J=8.8 Hz, 2H), 3.87 (t, J=7.6 Hz, 4H), 2.12-2.19 (m, 2H).

Step 2: A mixture of 1-((4-nitrophenyl)sulfonyl)azetidine (8.8 g, 36.3 mmol) and Pd/C (0.8 g) in methanol (50 mL) was stirred at room temperature for 12 hr under hydrogen atmosphere. The catalyst Pd/C was filtered, and the filtrate was concentrated to afford 4-(azetidin-1-ylsulfonyl) aniline (7.3 g, 94%) as a light pink solid. LCMS (ES⁺): m/z calculated for C₉H₁₂N₂O₂S: 212.1; found: 213.0 [M+H]. ¹H NMR (400 MHz, CDCl₃) δ 7.63 (d, J=8.4 Hz, 2H), 6.74 (d, J=8.8 Hz, 2H), 4.17 (br, 2H), 3.73 (t, J=7.6 Hz, 4H), 2.03-2.07 (m, 2H).

Step 3: A solution of 4-(azetidin-1-ylsulfonyl)aniline (2.8 g, 13.2 mmol) and NH₄SCN (3.0 g, 39.6 mmol) in acetic acid (30 mL) was stirred at room temperature for 1 h followed by the addition of a solution of Br₂ (2.1 g, 13.2 mmol) in acetic acid (5 mL) dropwise. The resulting mixture was stirred at room temperature for 12 hr. After this time, acetic acid was removed under reduced pressure. The residue was diluted with EtOAc and Sat. aqueous NaHCO₃ solution. The precipitates formed was filtered, washed with water, and dried to 6-(azetidin-1-ylsulfonyl)benzo [d]thiazol-2-amine as a yellow solid (1.9 g, 53%). LCMS (ES⁺): m/z calculated for C₁₀H₁₁N₃O₂S₂: 269.0; found: 270.2 [M+H]. ¹H NMR (400 MHz, DMSO-d6) δ 8.17 (d, J=1.6 Hz, 1H), 8.01 (br, 2H), 7.61-7.59 (dd, J=8.4 Hz, 2.0 Hz, 1H), 7.51 (d, J=8.4 Hz, 1H), 3.63 (t, J=7.6 Hz, 4H), 1.91-2.00 (m, 2H).

### Intermediate 22: 6-(pyrrolidine-1-sulfonyl)-1,3-benzothiazol-2-amine

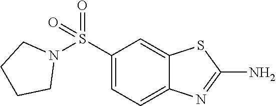 [SMILES: Nc1nc2ccc(S(=O)(=O)N3CCCC3)cc2s1]

This compound can be made as described for intermediate 21 (6-(azetidin-1-ylsulfonyl)benzo [d]thiazol-2-amine) by substituting pyrrolidine (CAS No. 123-75-1) for azetidine in Step 1. LCMS (ES+): m/z calculated for C₁₁H₁₃N₃O₃S₂: 283.4; found: 284.4 [M+H]. ¹H NMR (400 MHz, DMSO-d6) δ 8.21 (d, J=1.55 Hz, 1H), 7.84 (dd, J=7.50, 1.46 Hz, 1H), 7.74 (d, J=7.51 Hz, 1H), 7.11 (d, J=6.94 Hz, 1H), 6.94 (d, J=6.95 Hz, 1H), 3.21-3.11 (m, 5H), 1.76-1.66 (m, 5H).

### Intermediate 23: 6-(morpholine-4-sulfonyl)-1,3-benzothiazol-2-amine

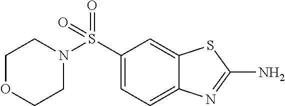 [SMILES: Nc1nc2ccc(S(=O)(=O)N3CCOCC3)cc2s1]

This compound can be made as described for intermediate 21 (6-(azetidin-1-ylsulfonyl)benzo [d]thiazol-2-amine) by substituting morpholine (CAS No. 5117-12-4) for azetidine in Step 1. LCMS (ES+): m/z calculated for C₁₁H₁₃N₃O₃S₂: 299.4; found: 300.4 [M+H]. ¹H NMR (400 MHz, DMSO-d6) δ 8.28-8.24 (m, 1H), 7.83-7.75 (m, 2H), 7.09 (d, J=6.96 Hz, 1H), 6.94 (d, J=6.96 Hz, 1H), 3.68 (t, J=7.11 Hz, 4H), 2.96 (t, J=7.06 Hz, 4H).

### Intermediate 24: 6-{2-oxa-6-azaspiro[3.3]heptane-6-sulfonyl}-1,3-benzothiazole-2-amine

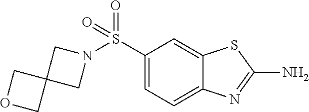 [SMILES: Nc1nc2ccc(S(=O)(=O)N3CC4(COC4)C3)cc2s1]

This compound can be made as described for intermediate 21 (6-(azetidin-1-ylsulfonyl)benzo [d]thiazol-2-amine) by substituting 2-oxa-6-azaspiro[3.3]heptane hemioxalate (CAS No. 1045709-32-7) for azetidine in Step 1. LCMS (ES⁺): m/z calculated for C₁₂H₁₃N₃O₃S₂: 311.0; found: 312.1 [M+H]. ¹H NMR (400 MHz, DMSO-d6) δ 8.17 (d, J=1.6 Hz, 1H), 8.02 (br, 2H), 7.59 (dd, J=8.4 Hz, 2.0 Hz, 1H), 7.48 (d, J=8.8 Hz, 1H), 4.42 (s, 4H), 3.85 (s, 4H).

### Intermediate 24′: 6-(piperidine-1-sulfonyl)-1,3-benzothiazol-2-amine

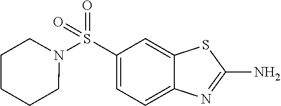 [SMILES: Nc1nc2ccc(S(=O)(=O)N3CCCCC3)cc2s1]

This compound can be made as described for intermediate 21 (6-(azetidin-1-ylsulfonyl)benzo [d]thiazol-2-amine) by substituting piperidine (CAS No. 110-89-4) for azetidine in Step 1. LCMS (ES+): m/z calculated for C₁₂H₁₅N₃O₃S₂: 297.4; found: 298.4 [M+H]. ¹H NMR (400 MHz, DMSO-d6) δ 8.21 (d, J=1.51 Hz, 1H), 7.81 (dd, J=7.51, 1.65 Hz, 1H), 7.76 (d, J=7.44 Hz, 1H), 7.09 (d, J=6.94 Hz, 1H), 6.94 (d, J=6.95 Hz, 1H), 2.97 (t, J=7.05 Hz, 4H), 1.65-1.56 (m, 4H), 1.49-1.39 (m, 2H).

### Intermediate 25: 1-[(2-amino-1,3-benzothiazol-6-yl)sulfonyl]pyrrolidin-3-ol

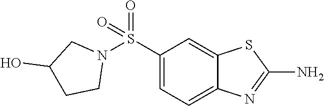 [SMILES: Nc1nc2ccc(S(=O)(=O)N3CCC(O)C3)cc2s1]

Step 1: To a mixture of 4-nitrobenzene-1-sulfonyl chloride (CAS No. 98-74-8, 7.0 g, 31.57 mmol), Na₂CO₃ (4.56 g, 43.05 mmol), pyrrolidin-3-ol (CAS No. 40499-83-0, 2.5 g, 28.70 mmol) in MeCN (15 mL) was stirred at 0° C. for 2 h. The reaction was quenched with water and extracted with ethyl acetate. The organic layer was washed with brine, dried over Na₂SO₄, and concentrated under reduce pressure. The crude product was purified through silica gel column chromatography (dichloromethane/methanol=20/1) to afford 1-((4-nitrophenyl) sulfonyl) pyrrolidin-3-ol as a white solid (6.0 g, 77%). LC/MS (ES⁺) calcd for C₁₀H₁₂N₂O₅S: 272.3; found: 273.1 [M+H]. ¹H NMR (400 MHz, CDCl₃) δ 8.35-8.40 (m, 2H), 8.01-8.05 (m, 2H), 4.42-4.47 (m, 1H), 3.47-3.54 (m, 2H), 3.33-3.41 (m, 1H), 3.28-3.33 (m, 2H), 1.94-2.06 (m, 1H), 184-1.93 (m, 1H), 1.49 (d, J=3.6 Hz, 1H).

Step 2: A mixture of 1-((4-nitrophenyl)sulfonyl)pyrrolidin-3-ol (6.0 g, 22.04 mmol), iron powder (6.17 g, 1.10 mol) and NH₄Cl(5.9 g, 1.10 mol) in EtOH/H₂O (2/1, 90 mL) was stirred at 80° C. for 2 hr. The resulting mixture was filtered through Celite, and the filter cake was rinsed with EtOH. The combined filtrate was concentrated under reduced pressure to remove most of the ethanol, and then extracted with DCM. The organic phase was dried over Na₂SO₄, concentrated, and purified through silica gel column chromatography (DCM/MeOH=50/1) to afford 1-((4-aminophenyl)sulfonyl)pyrrolidin-3-olas a white solid (5.0 g, 94%). LC/MS (ES+) calcd for C₁₀H₁₄N₂O₃S: 242.3; found: 243.1[M+H]. ¹H NMR (400 MHz, DMSO-d₆) δ 7.40 (d, J=8.4 Hz, 2H), 6.62 (d, J=8.8 Hz, 2H), 6.00 (s, 2H), 4.89 (d, J=4.0 Hz, 1H), 4.09-4.15 (m, 1H), 3.14-3.18 (m, 1H), 3.11 (dd, J=5.6, 8.4 Hz, 2H), 2.85-2.90 (m, 1H), 1.65-1.77 (m, 1H), 1.53-1.61 (min, 1H).

Step 3: To a solution of 1-((4-aminophenyl)sulfonyl)pyrrolidin-3-ol (1.5 g, 6.20 mmol) in MeCN (20 mL) was added benzoyl isothiocyanate (1.1 g, 6.51 mmol) at room temperature. The resulting mixture was stirred at room temperature for 0.5 h. The reaction mixture was filtered, and the filter cake was washed with water, dried to afford N-((4-((3-hydroxypyrrolidin-1-yl)sulfonyl)phenyl)carbamothioyl)benzamideas a white solid (2.4 g, 95%). LC/MS (ES⁺) calcd for C₁₈H₁₉N₃O₄S₂: 405.5; found: 406.1[M+H]. ¹H NMR (400 MHz, DMSO-d₆) δ 12.79 (s, 1H), 11.71 (s, 1H), 8.04 (d, J=8.8 Hz, 2H), 7.99 (d, J=7.6 Hz, 2H), 7.84 (d, J=8.4 Hz, 2H), 7.67 (t, J=7.4 Hz, 1H), 7.55 (t, J=7.8 Hz, 2H), 4.90 (br, 1H), 4.14-4.20 (m, 1H), 3.21-3.31 (m, 3H), 3.01-3.06 (m, 1H), 1.70-1.80 (m, 1H), 1.61-1.69 (m, 1H).

Step 4: A mixture of N-((4-((3-hydroxypyrrolidin-1-yl)sulfonyl)phenyl)carbamothioyl) benzamide (1.0 g, 2.47 mmol) and aq. NaOH (2N, 6 mL, 12.33 mmol) in MeOH (10 mL) was stirred at 70° C. for 1 h. The reaction mixture was concentrated under reduce pressure. The crude product was purified through silica gel column chromatography (dichloromethane/methanol=30/1) to 1-(4-((3-hydroxypyrrolidin-1-yl)sulfonyl)phenyl)thiourea as a white solid (800 mg, 89%). LC/MS (ES⁺) calcd for C₁₁H₁₅N₃O₃S₂: 301.4; found: 302.1 [M+H]. ¹H NMR (400 MHz, DMSO-d₆) δ 10.09 (s, 1H), 7.77 (d, J=8.8 Hz, 2H), 7.71 (d, J=8.8 Hz, 2H), 4.91 (d, J=3.2 Hz, 1H), 4.12-4.18 (m, 1H), 3.15-3.26 (m, 3H), 2.96-3.01 (m, 1H), 1.68-1.78 (m, 1H), 1.57-1.66 (m, 1H).

Step 5: A mixture of 1-(4-((3-hydroxypyrrolidin-1-yl)sulfonyl)phenyl)thiourea (800 mg, 2.65 mmol) and Br₂ (467 mg, 2.92 mmol) in CHCl₃ (10 mL) was stirred at 70° C. for 12 hr. The reaction mixture was concentrated under reduce pressure and diluted with dichloromethane. The solution was neutralized with aq. NH₃.H₂O solution. The organic phase was concentrated under reduce pressure and purified through silica gel column chromatography (dichloromethane/methanol=30/1) to afford 1-((2-aminobenzo[d]thiazol-6-yl)sulfonyl)pyrrolidin-3-ol as a white solid (430 mg, 54%). LC/MS (ES⁺) calcd for C₁₁H₁₃N₃O₃S₂: 299.4; found: 300.1 [M+H]. 1H NMR (400 MHz, DMSO-d₆) δ 8.15 (d, J=2.0 Hz, 1H), 7.94 (s, 2H), 7.59 (dd, J=1.8, 8.6 Hz, 1H), 7.43 (d, J=8.8 Hz, 11), 4.88 (d, J=3.6 Hz, 1H), 4.10-4.16 (m, 1H), 3.15-3.26 (m, 3H), 2.95-3.00 (m, 1H), 1.67-1.77 (m, 1H), 1.55-1.64 (m, 1H).

### Intermediate 26: (3R)-1-[(2-amino-1,3-benzothiazol-6-yl)sulfonyl]pyrrolidin-3-ol

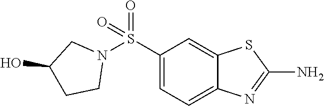 [SMILES: Nc1nc2ccc(S(=O)(=O)N3CC[C@@H](O)C3)cc2s1]

This compound can be made as described for Intermediate 50: 1-[(2-amino-1,3-benzothiazol-6-yl)sulfonyl]pyrrolidin-3-ol, by substituting (3R)-3-Pyrrolidinol (CAS No. 2799-21-5) for pyrrolidin-3-ol, Step 1. LC/MS (ES⁺) calcd for C₁₁H₁₃N₃O₃S₂: 299.4; found: 300.1 [M+H]. ¹H NMR (400 MHz, DMSO-d₆) δ 8.12 (d, J=2.0 Hz, 1H), 7.96 (s, 2H), 7.60 (dd, J=1.8, 8.6 Hz, 1H), 7.40 (d, J=8.8 Hz, 1H), 4.88 (d, J=3.6 Hz, 1H), 4.10-4.16 (m, 1H), 3.15-3.26 (m, 3H), 2.95-3.00 (m, 1H), 1.67-1.77 (m, 1H), 1.55-1.64 (m, 1H).

### Intermediate 27: (3S)-1-[(2-amino-1,3-benzothiazol-6-yl)sulfonyl]pyrrolidin-3-ol

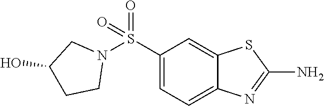 [SMILES: Nc1nc2ccc(S(=O)(=O)N3CC[C@H](O)C3)cc2s1]

This compound can be made as described for Intermediate 50: 1-[(2-amino-1,3-benzothiazol-6-yl)sulfonyl]pyrrolidin-3-ol, by substituting (3S)-3-pyrrolidinol (CAS No. 100243-39-8) for pyrrolidin-3-ol, Step 1. LC/MS (ES⁺) calcd for C₁₁H₁₃N₃O₃S₂: 299.4; found: 300.1 [M+H]. ¹H NMR (400 MHz, DMSO-d₆) δ 8.12 (d, J=2.0 Hz, 1H), 7.92 (s, 2H), 7.60 (dd, J=1.8, 8.6 Hz, 1H), 7.40 (d, J=8.8 Hz, 1H), 4.90 (d, J=3.6 Hz, 1H), 4.10-4.16 (m, 1H), 3.15-3.26 (m, 3H), 2.95-3.00 (m, 1H), 1.67-1.77 (m, 1H), 1.55-1.64 (m, 1H).

### Intermediate 28: 6-(pyrrolidine-1-sulfonyl)-1-benzothiophen-2-amine

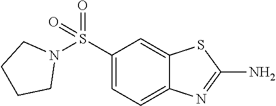 [SMILES: Nc1nc2ccc(S(=O)(=O)N3CCCC3)cc2s1]

Step 1: To a solution of methyl 4-amino-2-fluorobenzoate (64.0 g, 378 mmol) in concentrated aq. hydrochloric acid (640 mL) was added aqueous NaNO₂ solution (28.7 g, 416 mmol, in 50 mL) at 10° C. After stirring at 10° C. for 20 min, the mixture was added dropwise at 0° C. into a solution of CuCl (375 mg, 3.8 mmol) in HOAc (500 mL) which was saturated with SO₂ gas. The resulting mixture was warmed up to room temperature, and stirred for 2 h. The reaction mixture was poured into ice-water and extracted with ethyl acetate. The combined organic phase was washed with saturated aq. NaHCO₃ solution and then brine, dried over Na₂SO₄, and concentrated under reduced pressure to afford methyl 4-(chlorosulfonyl)-2-fluorobenzoateas a brown oil (86.1 g, 90%). ¹H NMR (400 MHz, CDCl₃) δ 8.18 (dd, J=6.8 Hz, 8.4 Hz, 1H), 7.89 (dd, J=2.0 Hz, 8.4 Hz, 1H), 7.83 (dd, J=2.0 Hz, 8.8 Hz, 1H), 4.00 (s, 3H).

Step 2: To a solution of pyrrolidine (26.6 g, 375 mmol) and DIPEA (88 g, 682 mmol) in DCM (720 mL) was added methyl 4-(chlorosulfonyl)-2-fluorobenzoate (86.0 g, 341 mmol) at room temperature. The resulting mixture was stirred at room temperature for 1 h. After the reaction was completed, the reaction mixture was diluted with DCM, washed with hydrochloric acid (1 N), saturated aqueous NaHCO₃ solution, and brine respectively. The organic layer was dried over Na₂SO₄, and concentrated under reduced pressure to afford methyl 2-fluoro-4-(pyrrolidin-1-ylsulfonyl)benzoate as a light red solid (90.0 g, 91%). LC/MS (ES⁺) calcd for C₁₂H₁₄FNO₄S: 287.1; found: 287.9 [M+H]. ¹H NMR (400 MHz, CDCl₃) δ 8.08 (dd, J=6.8 Hz, 8.0 Hz, 1H), 7.65 (dd, J=1.4 Hz, 8.2 Hz, 1H), 7.61 (dd, J=1.4 Hz, 9.8 Hz, 1H), 3.97 (s, 3H), 3.31-3.24 (m, 4H), 1.83-1.77 (m, 4H).

Step 3: To a suspension of LiAlH₄ (19.8 g, 522 mmol) in THE (300 mL) was added a solution of methyl 2-fluoro-4-(pyrrolidin-1-ylsulfonyl)benzoate (50.0 g, 174 mmol) in THE (200 mL) dropwise at −50 OC under nitrogen atmosphere. After stirring at room temperature for 1 h, the reaction was quenched with saturated aq. NH₄Cl solution, and then celite was added. The mixture was extracted with DCM. The organic phase was washed with brine, dried over Na₂SO₄, and concentrated under reduced pressure to afford [(2-fluoro-4-(pyrrolidin-1-ylsulfonyl) phenyl)methanol as a white solid (42.6 g, 94%). LC/MS (ES⁺) calcd for C₁₁H₁₄FNO₃S: 259.1; found: 260.0 [M+H]. ¹H NMR (400 MHz, CDCl₃) δ 7.66 (t, J=7.4 Hz, 1H), 7.60 (dd, J=1.6 Hz, 8.0 Hz, 1H), 7.48 (dd, J=1.6 Hz, 9.2 Hz, 1H), 4.84 (s, 2H), 3.27-3.22 (m, 4H), 1.80-1.75 (m, 4H).

Step 4: To a suspension of Dess-Martin reagent (14.0 g, 330 mmol) in DCM (450 mL) was added a solution of (2-fluoro-4-(pyrrolidin-1-ylsulfonyl)phenyl)methanol (42.6 g, 165 mmol) in DCM (400 mL) at 0° C. After stirring for 2 h, the reaction was quenched with water; the resulting mixture was filtered through ceilite, and the filter cake was rinsed with DCM. The combined organic phase was washed with brine, dried over Na₂SO₄, and concentrated under reduced pressure to afford 2-fluoro-4-(pyrrolidin-1-ylsulfonyl)benzaldehyde as a white solid (36.3 g, 86%). ¹H NMR (400 MHz, CDCl₃) δ 10.41 (s, 1H), 8.03 (dd, J=6.4 Hz, 8.0 Hz, 1H), 7.72 (d, J=8.4 Hz, 1H), 7.67 (dd, J=6.4 Hz, 9.2 Hz, 1H), 3.32-3.27 (m, 4H), 1.84-1.80 (m, 4H).

Step 5: To a solution of 2-fluoro-4-(pyrrolidin-1-ylsulfonyl)benzaldehyde (44.0 g, 171 mmol) in DMF (440 mL) were added methyl 2-mercaptoacetate (20.0 g, 188 mmol) and K₂CO₃ (47.2 g, 342 mmol) at room temperature. After being stirred 12 h, the reaction was quenched with water (2200 ml) with ice-water bath cooling, and then stirred for 1 h. The resulting mixture was filtered; the filter cake was washed with water and triturated with ethanol to afford methyl 6-(pyrrolidin-1-ylsulfonyl)benzo[b]thiophene-2-carboxylate as a white solid (47.3 g, 85%). LC/MS (ES⁺) calcd for C₁₄H₁₅NO₄S₂: 325.0; found: 325.9 [M+H]. ¹H NMR (400 MHz, DMSO-d6) δ 8.66 (s, 1H), 8.33 (s, 1H), 8.24 (d, J=8.4 Hz, 1H), 7.85 (dd, J=0.8 Hz, 8.4 Hz, 1H), 3.92 (s, 3H), 3.23-3.16 (m, 4H), 1.67-1.61 (m, 4H).

Step 6: To a solution of methyl 6-(pyrrolidin-1-ylsulfonyl)benzo[b]thiophene-2-carboxylate (47.3 g, 145 mmol) in THF (900 mL) and water (300 mL) was added LiOH.H₂O (18.3 g, 437 mmol) at room temperature. The resulting mixture was stirred at room temperature for 12 h. After this time, THF was removed under reduced pressure; the aqueous phase was acidified with hydrochloric acid (1 N) to pH-4-5, and the resulting mixture was stirred at 0° C. for 1 h. The suspension was filtered; the filter cake was washed with water and dried to afford 6-(pyrrolidin-1-ylsulfonyl)benzo[b]thiophene-2-carboxylic acid as a white solid (44.0 g, 97%). LC/MS (ES⁺) calcd for C₁₃H₁₃NO₄S₂: 311.0; found: 311.9 [M+H]. ¹H NMR (400 MHz, DMSO-d6) δ 13.77 (br, 1H), 8.63 (s, 1H), 8.22 (s, 1H), 8.24 (d, J=8.8 Hz, 1H), 7.83 (dd, J=1.6 Hz, 8.4 Hz, 1H), 3.23-3.16 (m, 4H), 1.66-1.61 (m, 4H).

Step 7: To a stirred suspension of 6-(pyrrolidin-1-ylsulfonyl)benzo[b]thiophene-2-carboxylic acid (20.0 g, 64.3 mmol) in toluene was added TEA (9.7 g, 94.4 mmol) and n (26.0 g, 94.4 mmol) at ambient temperature. The resulting mixture was heated to 80° C. under nitrogen atmosphere, and stirred for 1 h. t-BuOH (6.0 g, 90 mmol) was added slowly to the reaction mixture; the resulting mixture was heated to 100° C., and stirred 12 h. After cooled down to room temperature, the reaction mixture was washed with water and then brine, dried over Na₂SO₄, and concentrated under reduced pressure to give a residue which was purified through silica gel column chromatography (hexane/DCM=5/1˜1/2) to afford tert-butyl (6-(pyrrolidin-1-ylsulfonyl)benzo[b]thiophen-2-yl)carbamate as a white solid (19.6 g, 80%). LC/MS (ES⁺) calcd for C₁₇H₂₂N₂O₄S₂: 382.1; found: 383.0 [M+H]. ¹H NMR (400 MHz, CDCl₃) δ 8.19 (s, 1H), 7.71 (dd, J=1.6 Hz, 8.4 Hz, 1H), 7.63 (d, J=8.4 Hz, 1H), 6.77 (s, 1H), 3.28-3.24 (m, 4H), 1.76-1.71 (m, 4H), 1.56 (s, 9H).

Step 8: To a solution of tert-butyl (6-(pyrrolidin-1-ylsulfonyl)benzo[b]thiophen-2-yl)carbamate (2.0 g, 5.2 mmol) in DCM (20 mL) was added TFA (6.0 mL) at room temperature. After stirring for 2 hrs, the reaction mixture was added slowly into saturated aqueous NaHCO₃ solution (100 mL) at 0° C., and extracted with DCM. The combined organic phase was washed with brine, dried over Na_{2S}O₄, and concentrated under reduced pressure to give a residue which was purified through silica gel column chromatography (hexane/DCM=5/1˜100% DCM) to afford 6-(pyrrolidin-1-ylsulfonyl)benzo[b]thiophen-2-amine as a pink solid (1.1 g, 69%). LC/MS (ES⁺) calcd for C₁₂H₁₄N₂O₂S₂: 282.1; found: 282.9 [M+H]. ¹H NMR (400 MHz, CDCl₃) δ 8.03 (d, J=1.2 Hz, 1H), 7.64 (dd, J=1.8 Hz, 8.2 Hz, 1H), 7.45 (d, J=8.4 Hz, 1H), 6.29 (s, 1H), 4.38 (br, 2H), 3.27-3.21 (m, 4H), 1.76-1.72 (m, 4H).

### Preparation of Carboxylic Acid Intermediates

The following acids shown in Table 2 were used in preparing the compounds of the invention. They are either commercially available or can be prepared by known synthetic procedures. CAS registry numbers are provided for each.

TABLE 2
Commercial carboxylic acids

| Int. | | | |
|---|---|---|---|
| No. | Structure | Name | CAS No. |
| 29 | 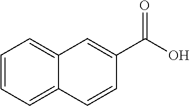 [SMILES: O=C(O)c1ccc2ccccc2c1] | 2-Naphthalenecarboxylic acid | 93-09-4 |
| 30 | 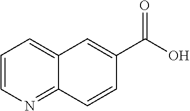 [SMILES: O=C(O)c1ccc2ncccc2c1] | 6-Quinolinecarboxylic acid | 10349-57-2 |
| 31 | 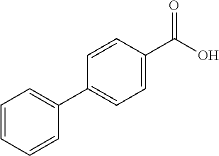 [SMILES: O=C(O)c1ccc(-c2ccccc2)cc1] | 4-Biphenylcarboxylic acid | 92-92-2 |
| 32 | 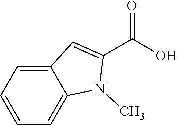 | 1-Methylindole-2-carboxylic acid | 16136-58-6 |
| 33 | 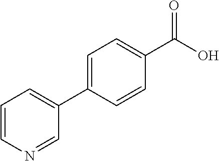 [SMILES: O=C(O)c1ccc(-c2cccnc2)cc1] | 4-(3-Pyridinyl) benzoic acid | 4385-75-5 |
| 34 | 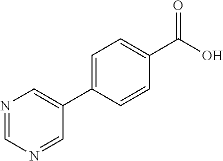 [SMILES: O=C(O)c1ccc(-c2cncnc2)cc1] | 4-(5-Pyrimidyl)benzoic acid | 216959-91-0 |
| 35 | 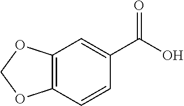 [SMILES: O=C(O)c1ccc2c(c1)OCO2] | 1,3-Benzodioxole-5-carboxylic acid | 94-53-1 |
| 36 | 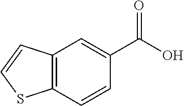 [SMILES: O=C(O)c1ccc2sccc2c1] | Benzothiophene-5-carboxylic acid | 2060-64-2 |

### Intermediate 37: 3-[3-(Morpholin-4-yl)ethoxy]naphthalene-2-carboxylic Acid

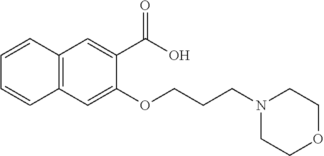 [SMILES: O=C(O)c1cc2ccccc2cc1OCCCN1CCOCC1]

Step 1: To a solution of methyl 3-hydroxy-2-naphthoate (CAS No. 92-70-6, 560 mg, 2.7 mmol), 3-morpholinopropan-1-ol (CAS No. 441-30-9, 800 mg, 5.5 mmol), and PPh₃ (1.44 g, 5.5 mmol) in THF (5.6 mL) at −5° C. was added dropwise DIAD (1.11 g, 5.5 mmol). The resulting mixture was stirred at RT for 12 h. After the solvent was removed, the residue was purified through column chromatography (eluent: DCM/MeOH from 100:1 to 40:1) to afford 3-[3-(morpholin-4-yl)ethoxy]naphthalene-2-carboxylic acid (732 mg, 80%) as a colorless oil. LC/MS (ES⁺): 330.5 [M+H]. ¹H NMR (400 MHz CDCl₃): δ 8.29 (s, 1H), 7.81 (d, J=8.13 Hz, 1H), 7.71 (d, J=8.22 Hz, 1H), 7.55-7.47 (m, 1H), 7.40-7.33 (m, 1H), 7.19 (s, 1H), 4.19 (t, J=6.13 Hz, 2H), 3.94 (s, 3H), 3.80-3.73 (m, 4H), 2.70 (t, J=7.6 Hz, 2H), 2.64-2.56 (m, 4H), 2.15-2.08 (m, 2H)

Step 2: A solution of methyl 3-(3-morpholinopropoxy)-2-naphthoate (400 mg, 1.2 mmol) and LiOH.H₂O (87 mg, 2.1 mmol) in methanol/water (2 mL/1.6 mL) was stirred at RT for 1 h. The reaction mixture was diluted with water and extracted with ethyl acetate. The aqueous phase was adjusted to pH 6-7 with diluted hydrochloric acid (1.0 N), and extracted with DCM/MeOH (3:1, 4×10 mL). The organic layer was dried over Na₂SO₄ and concentrated under vacuum to afford the 3-[3-(morpholin-4-yl)ethoxy]naphthalene-2-carboxylic acid (240 mg, 63%) as white foam. LC/MS (ES; found: 316.3 [M+H]. ¹H NMR (400 MHz, CDCl₃): δ 8.53 (s, 1H), 7.82 (d, J=8.17 Hz, 1H), 7.71 (d, J=8.24 Hz, 1H), 7.54-7.50 (m, 1H), 7.41-7.37 (m, 1H), 7.23 (s, 1H), 4.35 (t, J=5.94 Hz, 2H), 3.91-3.82 (m, 4H), 2.88 (t, J=6.79 Hz, 2H), 2.81-2.73 (m, 4H), 2.27-2.20 (m, 2H).

### Intermediate 38: 3-[2-(Morpholin-4-yl)ethoxy]naphthalene-2-carboxylic Acid

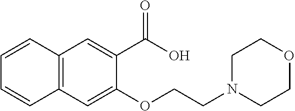 [SMILES: O=C(O)c1cc2ccccc2cc1OCCN1CCOCC1]

This compound can be prepared as described for Intermediate 62: 3-[3-(Morpholin-4-yl)ethoxy] naphthalene-2-carboxylic acid by substituting 3-morpholinopropan-1-ol step 1, with 4-morpholineethanol (CAS No. 622-40-2). LC/MS (ES; found: 303.3 [M+H]. ¹H NMR (400 MHz, Chloroform-d): δ 8.43 (d, J=2.2 Hz, 1H), 7.94-7.87 (m, 1H), 7.78-7.72 (m, 1H), 7.56 (ddd, J=8.5, 6.6, 1.1 Hz, 1H), 7.53-7.44 (m, 2H), 4.36 (t, J=6.4 Hz, 2H), 3.69 (t, J=6.0 Hz, 4H), 2.70 (t, J=6.5 Hz, 2H), 2.59-2.44 (m, 4H).

### Intermediate 39: 3-[4-(Morpholin-4-yl)butoxy]naphthalene-2-carboxylic Acid

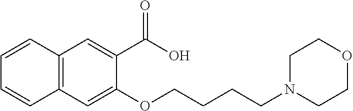 [SMILES: O=C(O)c1cc2ccccc2cc1OCCCCN1CCOCC1]

This compound can be prepared as described for Intermediate 37: 3-[3-(Morpholin-4-yl)ethoxy] naphthalene-2-carboxylic acid by substituting 3-morpholinopropan-1-ol step 1, with 4-morpholinebutanol (CAS No. 5835-79-0). LC/MS (ES; found: 330.4 [M+H]. ¹H NMR (400 MHz, CDCl₃): δ 8.47-8.42 (m, 1H), 7.94-7.87 (m, 1H), 7.75 (dt, J=7.9, 1.9 Hz, 1H), 7.56 (ddd, J=8.5, 6.8, 1.1 Hz, 1H), 7.53-7.42 (m, 2H), 4.05 (t, J=6.1 Hz, 2H), 3.78 (t, J=6.0 Hz, 4H), 2.56-2.43 (m, 6H), 1.77-1.68 (m, 2H), 1.63-1.53 (m, 2H).

### Intermediate 40: 1-methyl-5-[2-(morpholin-4-yl)ethoxy]-1H-indole-6-carboxylic Acid

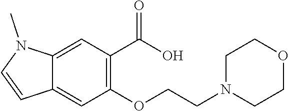 [SMILES: Cn1ccc2cc(OCCN3CCOCC3)c(C(=O)O)cc21]

Step 1: To a mixture of 2-hydroxy-4-methylbenzoic acid (80 g, 0.5 mol) and K₂CO₃ (218 g, 1.58 mol) in DMF (300 mL) was added iodomethane (224 g, 1.5 mol) dropwise at 0° C. The resulting mixture was stirred at 40° C. for 12 h. The reaction mixture was filtered, and the filtrate was partitioned into water (1,500 ml) and ethyl acetate (800 ml). The organic layer was collected, washed with water (300 ml×2) and brine (300 ml), dried over Na₂SO₄, and concentrated under reduce pressure to give a crude product which was purified through silica gel flash column chromatography (cyclohexane/ethyl acetate=10/1) to afford methyl 2-methoxy-4-methylbenzoate as a yellow oil (82 g, 86%). LC/MS (ES⁺) calcd for C₁₀H₁₂O₃: 180.1; found: 181.0 [M+H]. ¹H NMR (400 MHz, CDCl₃): δ 7.72 (d, J=8.0 Hz, 1H), 6.78-6.79 (m, 2H), 3.89 (s, 3H), 3.86 (s, 3H), 2.38 (s, 3H).

Step 2: To a mixture of methyl 2-methoxy-4-methylbenzoate (82 g, 0.46 mol) in acetic acid/acetic anhydride (1/1, 400 mL) was added nitric acid (128 mL) dropwise at 0° C. and then raised to 40° C. slowly and stirred for 12 h. The resulting mixture was poured into ice water and extracted with DCM. The organic phases were washed with brine, dried over Na₂SO₄ and concentrated under reduce pressure. The crude product was purified through silica gel flash column chromatography (cyclohexane/DCM/ethyl acetate=8/2/1) to afford methyl 2-methoxy-4-methyl-5-nitrobenzoate as an off-white solid (65 g, 63%). ¹H NMR (400 MHz, CDCl₃): δ 8.62 (s, 1H), 6.86 (s, 1H), 4.00 (s, 3H), 3.91 (s, 3H), 2.71 (s, 3H).

Step 3: A mixture of methyl 2-methoxy-4-methyl-5-nitrobenzoate (65 g, 0.29 mol) and DMF-DMA (103.7 g, 0.87 mol) in DMF (50 mL) was heated to 115° C., and stirred for 3 h. The reaction mixture was concentrated under reduced pressure to give a crude product which was triturated with diethyl ether to afford methyl 4-(2-(dimethylamino)vinyl)-2-methoxy-5-nitrobenzoate as a red solid (73 g, 90%). ¹H NMR (400 MHz, DMSO-d₆): δ 8.58 (s, 1H), 7.09 (d, J=13.6 Hz, 1H), 6.82 (s, 1H), 6.12 (d, J=13.6 Hz, 1H), 3.98 (s, 3H), 3.87 (s, 3H), 3.00 (s, 611H).

Step 4: A mixture of methyl 4-(2-(dimethylamino)vinyl)-2-methoxy-5-nitrobenzoate (43 g, 0.15 mol) and 10% Pd/C (4.3 g) in THF (80 mL) was stirred at room temperature under hydrogen atmosphere (balloon pressure) for 12 h. After this time, the Pd/C was filtered off, and the filter cake was rinsed with methanol. The combined filtrate was concentrated under reduce pressure to give a crude product was purified through silica gel flash column chromatography (cyclohexane/DCM/ethyl acetate=8/2/1) to afford methyl 5-methoxy-1H-indole-6-carboxylate as a white solid (21.9 g, 69%). LC/MS (ES⁺) calcd for C₁₁H₁₁NO₃: 205.1; found: 206.0 [M+H]. ¹H NMR (400 MHz, CDCl₃): δ 8.35 (br, 1H), 7.94 (s, 1H), 7.33-7.31 (m, 1H), 7.16 (s, 1H), 6.51-6.48 (m, 1H), 3.93 (s, 3H), 3.91 (s, 3H).

Step 5: A mixture of methyl 5-methoxy-1H-indole-6-carboxylate (21.9 g, 0.1 mol), MeONa (5.9 g, 0.11 mol), and MeI (16.5 g, 0.1 mol) in THF (50 mL) was stirred at 0° C. for 2 h. After completion, the reaction was quenched with water, and extracted with DCM, dried over Na₂SO₄, and concentrated under reduce pressure to give a crude product which was purified through silica gel flash column chromatography (cyclohexane/DCM/ethyl acetate=8/2/1) to afford methyl 5-methoxy-1-methyl-1H-indole-6-carboxylate as a white solid (20.6 g, 88%). LC/MS (ES⁺) calcd for C₁₂H₁₃NO₃: 219.1; found: 220.0 [M+H]. ¹H NMR (400 MHz, CDCl₃): δ 7.87 (s, 1H), 7.15 (d, J=2.8, 1H), 7.14 (s, 1H), 6.40 (dd, J=0.8 Hz, 2.8 Hz, 1H), 3.93 (d, J=1.6 Hz, 6H), 3.80 (s, 3H).

Step 6: To a solution of methyl 5-methoxy-1-methyl-1H-indole-6-carboxylate (7 g, 30 mmol) in DCM (50 mL) was added dropwise BBr₃ in DCM (1.0 N, 150 ml, 150 mmol) at −70° C. under nitrogen atmosphere. After stirring at −70° C. for 30 min, the reaction was quenched slowly with methanol (30 ml) −70° C., and then warmed to room temperature, stirred for an additional 30 min. The reaction mixture was partitioned between water and DCM, the organic phase was collected, and the aqueous phase was extracted with DCM (100 ml×2). The combined organic layer was washed with brine, dried over Na₂SO₄, and concentrated under reduce pressure to give a crude product which was purified through silica gel flash column chromatography (cyclohexane/ethyl acetate=10/1) to afford methyl 5-hydroxy-1-methyl-1H-indole-6-carboxylate as a white solid (1.6 g, 22%). LC/MS (ES⁺) calcd for C₁₁H₁₁NO₃: 205.1; found: 206.0 [M+H].

Step 7: A mixture of methyl 5-hydroxy-1-methyl-1H-indole-6-carboxylate (1.6 g, 7.8 mmol), 4-(2-chloroethyl)morpholine hydrochloride (1.7 g, 9.4 mmol), and cesium carbonate (7.6 g, 23.4 mmol) in DMF (20 mL) was stirred at 85° C. under nitrogen atmosphere for 3 h. The reaction mixture was filtered, and the filter cake was rinsed ethyl acetate. The combined filtrate was washed with water and then brine, dried over Na₂SO₄, and concentrated under reduce pressure to give a crude product was purified through silica gel flash column chromatography (DCM/MeOH/Et₃N=100/1/5%) to afford methyl 1-methyl-5-(2-morpholinoethoxy)-1H-indole-6-carboxylate as a white solid (2.1 g, 85%). LC/MS (ES⁺) calcd. for C₁₇H₂₂N₂O₄: 318.2; found: 319.3 [M+H]. ¹H NMR (400 MHz, CDCl₃): δ 7.86 (s, 1H), 7.16-7.14 (m, 2H), 6.41-6.38 (m, 1H), 4.21 (t, J=5.6 Hz, 2H), 3.91 (s, 3H), 3.80 (s, 3H), 3.77-3.73 (m, 4H), 2.88 (t, J=5.6 Hz, 2H), 2.66-2.62 (m, 4H).

Step 8: To a solution of methyl 1-methyl-5-(2-morpholinoethoxy)-1H-indole-6-carboxylate (2.1 g, 6.6 mmol) in THF/MeOH/H₂O (3/1/1, v/v/v, 20 mL) was added sodium hydroxide (0.66 g, 16.4 mmol), the resulting mixture was stirred at room temperature for 2 h. After the starting material disappeared, THF and methanol were removed under reduced pressure. The residue was acidified with hydrochloric acid (1N, 16.4 ml). The precipitate formed was collected through filtration and dried to afford 1-methyl-5-(2-morpholinoethoxy)-1H-indole-6-carboxylic acid as a yellow solid (750 mg, 37%). LC/MS (ES⁺) calcd for C₁₆H₂₀N₂O₄: 304.1; found: 305.1 [M+H].

### Intermediate 41: 1-methyl-5-[4-(morpholin-4-yl)butoxy]-1H-indole-6-carboxylic Acid

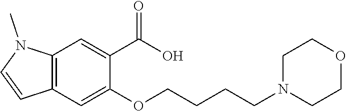 [SMILES: Cn1ccc2cc(OCCCCN3CCOCC3)c(C(=O)O)cc21]

This compound can be prepared as described above for Intermediate 40: 1-methyl-5-[2-(morpholin-4-yl)ethoxy]-1H-indole-6-carboxylic acid by substituting 2-(2-chloroethyl)morpholine with 4-(4-chlorobutyl)-morpholine (CAS No. 734495-59-1). LC/MS (ES⁺) calcd for C₁₈H₂₄N₂O₄: 332.4; found: 333.5 [M+H]. 1H NMR (400 MHz, DMSO-d6): δ 7.97 (s, 1H), 7.67 (d, J=1.79 Hz, 1H), 7.27-7.21 (m, 1H), 6.22 (dd, J=7.56, 1.60 Hz, 1H), 4.02 (t, J=7.09 Hz, 2H), 3.79 (s, 2H), 3.59 (t, J=7.11 Hz, 4H), 2.60 (t, J=7.11 Hz, 2H), 2.46 (t, J=7.11 Hz, 4H), 1.84 (p, J=7.12 Hz, 2H), 1.58 (p, J=7.04 Hz, 2H).

### Intermediate 42: 2-[2-(morpholin-4-yl)ethoxy]-4-phenylbenzoic Acid

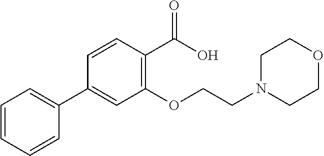 [SMILES: O=C(O)c1ccc(-c2ccccc2)cc1OCCN1CCOCC1]

Step 1: To a solution of methyl 4-bromo-2-methoxybenzoate (CAS No. 139102-34-4, 50 g, 204.02 mmol) and phenylboronic acid (29.85 g, 244.83 mmol) in toluene/EtOH/H₂O (195 ml/50 ml/25 ml) was added Na₂CO₃ (86.5 g, 810.1 mmol) and Pd(PPh₃)₄ (4.7 g, 4.1 mmol) under nitrogen atmosphere. The resulting mixture was heated to 100° C. under nitrogen atmosphere, and stirred for 4 h. After the completion of the reaction, the reaction mixture was filtered through celite, and the filter cake was rinsed with ethyl acetate. The organic phase was collected, and the aqueous phase was extracted with ethyl acetate. The combined organic phases were dried over Na₂SO₄ and concentrated under reduced pressure to give a residue which was purified through silica gel flash column chromatography (eluent: hexane/DCM=2/1˜1/1) to afford methyl 3-methoxy-[1,1′-biphenyl]-4-carboxylate as a yellow solid (49.22 g, 91%). LC/MS (ES⁺) calcd for C₁₅H₁₄O₃: 242.1; found: 243.0 [M+H]. ¹H NMR (400 MHz, DMSO-d6): δ 7.78-7.72 (m, 1H), 7.53-7.46 (m, 2H), 7.46-7.40 (m, 1H), 7.36 (d, J=1.2 Hz, 1H), 7.30 (dd, J=1.2 Hz, 12.0 Hz, 1H), 3.93 (s, 3H), 3.80 (s, 3H).

Step 2: To a solution of methyl 3-methoxy-[1,1′-biphenyl]-4-carboxylate (49.2 g, 203.1 mmol) in DCM (200 ml) was added dropwise a solution of BBr₃ (137.8 g, 550 mmol) in DCM (250 ml) with dry ice-acetone bath. The resulting mixture was stirred at −70° C. for 10 min, and then quenched with methanol (100 ml) slowly. The reaction mixture was washed with water (300 ml), and the aqueous phase was extracted with DCM. The combined organic phases were washed with brine, dried over Na₂SO₄, and concentrated under reduced pressure to give a residue which was purified through silica gel flash column chromatography (eluent: hexane/DCM=2/1) to afford methyl 3-hydroxy-[1,1′-biphenyl]-4-carboxylate as a white solid (44.62 g, 96%). LC/MS (ES⁺) calcd for C₁₄H₁₂O₃: 228.1; found: 229.0 [M+H]. ¹H NMR (400 MHz, DMSO-d6): δ 10.59 (s, 1H), 7.88-7.84 (m, 1H), 7.74-7.69 (m, 2H), 7.52-7.46 (m, 2H), 7.45-7.40 (m, 1H), 7.29-7.25 (m, 2H), 3.91 (s, 3H).

Step 3: To a stirred solution of methyl 3-hydroxy-[1,1′-biphenyl]-4-carboxylate (14.46 g, 63.35 mmol) and 4-(2-chloroethyl)morpholine HCl salt (14.06 g, 76.0 mmol) in DMF (240 mL) was added Cs₂CO₃ (61.9 g, 190.1 mmol), the resulting mixture was stirred at 85° C. under nitrogen atmosphere for 3 h. The reaction mixture was cooled down to room temperature and filtered; the filter cake was rinsed with ethyl acetate. The combine organic phase was washed with water and then brine, dried over Na₂SO₄, and concentrated under reduced pressure to give a residue which was purified through silica gel flash column chromatography (eluent: DCM/ethyl acetate=5/1) to afford methyl 3-(2-morpholinoethoxy)-[1,1′-biphenyl]-4-carboxylate as a yellow oil (21.69 g, 100%). LC/MS (ES⁺) calcd for C₂₀H₂₃NO₄: 341.2; found: 342.4 [M+H]. ¹H NMR (400 MHz, CDCl₃): δ 7.87 (d, J=8.0 Hz, 1H), 7.61-7.56 (m, 2H), 7.48-7.42 (m, 2H), 7.42-7.36 (m, 1H), 7.21 (dd, J=1.6 Hz, 8.0 Hz, 1H), 7.17 (d, J=1.6 Hz, 1H), 4.26 (t, J=5.8 Hz, 2H), 3.89 (s, 3H), 3.76-3.71 (m, 4H), 2.89 (t, J=5.6 Hz, 1H), 2.67-2.60 (m, 4H).

Step 4: To a solution of methyl 3-(2-morpholinoethoxy)-[1,1′-biphenyl]-4-carboxylate (24.46 g, 71.6 mmol) in THF/MeOH/H₂O (140 ml/40 ml/40 ml) was added NaOH (7.1 g, 179 mmol). After stirring at room temperature for 2 h, THF and methanol were removed under reduced pressure, and the remained aqueous phase was acidified with hydrochloric acid (1 N. The precipitate formed was collected through filtration, washed with water, dried to give 3-(2-morpholinoethoxy)-[1,1′-biphenyl]-4-carboxylic acid as a white solid (22.8 g, 88%). LC/MS (ES⁺) calcd for C₁₉H₂₁NO₄: 327.2; found: 328.3 [M+H]. 11H NMR (400 MHz, DMSO-d6): δ 12.09 (br, 2H), 7.81 (d, J=8.4 Hz, 1H), 7.79-7.74 (m, 2H), 7.54-7.48 (m, 2H), 7.46-7.41 (m, 2H), 7.38 (dd, J=1.6 Hz, 8.0 Hz, 1H), 4.65 (t, J=4.8 Hz, 2H), 3.96-3.84 (m, 4H), 3.61-3.56 (m, 2H), 3.37-3.20 (m, 4H).

### Intermediate 43: 2-[4-(morpholin-4-yl)butoxy]-4-phenylbenzoic Acid

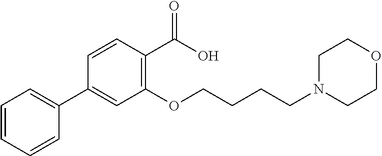 [SMILES: O=C(O)c1ccc(-c2ccccc2)cc1OCCCCN1CCOCC1]

This compound can be prepared as described above for Intermediate 42 by substituting 4-(2-chloroethyl)morpholine with 4-(4-chlorobutyl)-morpholine (CAS No. 734495-59-1) step 3. LC/MS (ES⁺) calcd for C21H25NO₄: 355.4; found: 355.5 [M+H]. ¹H NMR (400 MHz, DMSO-d6): δ 7.82 (d, J=7.49 Hz, 1H), 7.67-7.61 (m, 2H), 7.54 (dd, J=7.50, 1.45 Hz, 1H), 7.50-7.43 (m, 2H), 7.43-7.35 (m, 1H), 7.31 (d, J=1.46 Hz, 1H), 4.02 (t, J=7.08 Hz, 2H), 3.59 (t, J=7.08 Hz, 4H), 2.61 (t, J=7.10 Hz, 2H), 2.47 (t, J=7.11 Hz, 4H), 1.80 (p, J=7.12 Hz, 2H), 1.58 (p, J=7.23 Hz, 2H).

### Intermediate 44: 6-[2-(morpholin-4-yl)ethoxy]-1-benzothiophene-5-carboxylic Acid

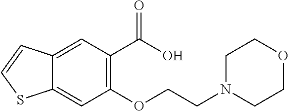 [SMILES: O=C(O)c1cc2ccsc2cc1OCCN1CCOCC1]

Step 1: To a solution of Br₂ (50 g, 0.311 mol) and KBr (92.6 g, 0.779 mol) in water (480 mL) was added 2-fluoro-4-methoxybenzaldehyde (CAS No. 331-64-6, 24 g, 0.16 mol) in portions at 0° C., the resulting mixture was stirred at room temperature for 4 h. The reaction mixture was filtered, and the filter cake was washed with water, dried to afford 5-bromo-2-fluoro-4-methoxybenzaldehyde as a white solid (28.9 g, 80%). LC/MS (ES⁺) calcd for C₈H₆BrFO₂: 232.0; found: 233.0 [M+H].

Step 2: To a mixture of 5-bromo-2-fluoro-4-methoxybenzaldehyde (20 g, 86 mmol) and K₂CO₃ (17.8, 129 mmol) in DMF (200 mL) was added methyl 2-mercaptoacetate (9.6 g, 90 mmol). The resulting mixture was stirred at 60° C. under N₂ for 30 min. The reaction mixture was quenched with water, and the precipitate formed was filtered. The filter cake was washed with water and dried to afford methyl 5-bromo-6-methoxybenzo[b]thiophene-2-carboxylate as a white solid (16.2 g, 63%). LC/MS (ES⁺) calcd for C₁₁H9BrO₃S: 300.0; found: 300.9 [M+H]. ¹H NMR (400 MHz, DMSO-d6): δ 8.29 (s, 1H), 8.08 (s, 1H), 7.81 (s, 1H), 3.94 (s, 3H), 3.87 (s, 3H).

Step 3: To a solution of methyl 5-bromo-6-methoxybenzo[b]thiophene-2-carboxylate (15 g, 49.8 mmol) in THF (200 mL) and water (80 mL) was added LiOH.H₂O (20.9 g, 498 mmol). The resulting mixture was stirred at 50° C. under N₂ for 3 h. The reaction mixture was cooled to room temperature, and acidified with hydrochloric acid (2 N) under ice-water bath. The precipitate formed was filtered and dried to afford 1-(2-aminobenzo[d]thiazol-7-yl)-3-phenylthiourea as a white solid (13.6 g, 95%). LC/MS (ES⁺) calcd for C₁₀H₇BrO₃S: 286.0; found: 286.9 [M+H]. ¹H NMR (400 MHz, DMSO-d6): δ 8.26 (s, 1H), 7.98 (s, 1H), 7.80 (s, 1H), 3.93 (s, 3H).

Step 4: To a suspension of 5-bromo-6-methoxybenzo[b]thiophene-2-carboxylic acid (20.7 g, 72 mmol) in quinoline (200 mL) was added copper powder (8.0 g, 126 mmol). The resulting mixture was stirred at 190° C. under N₂ for 3 h. After cooled to room temperature, the mixture was diluted with water, and acidified with hydrochloric acid (4 N) to adjust the pH to 3-4. The aqueous phase was extracted with ethyl acetate (80 ml×3); the combined organic phase was washed with brine, dried over Na₂SO₄, and concentrated under reduced pressure to give a residue which was purified through silica gel flash column chromatography (hexane/ethyl acetate=20/1) to afford 5-bromo-6-methoxybenzo[b]thiophene as a brown solid (11.3 g, 64%). LC/MS (ES⁺) calcd for C₉H₇BrOS: 241.9; found: 244.9. ¹H NMR (400 MHz, CDCl₃): δ 7.96 (s, 1H), 7.33 (s, 1H), 7.28 (d, J=5.6 Hz, 1H), 7.16 (d, J=5.2 Hz, 1H), 3.94 (s, 3H).

Step 5: To a solution of 5-bromo-6-methoxybenzo[b]thiophene (5.0 g, 20.6 mmol), diethyl oxalate (6.0 g, 41.1 mmol), and DMAP (7.5 g, 61.7 mol) in NMP (60 mL) was added Pd(PPh₃)₂Cl₂ (1.5 g, 2.1 mmol). The resulting mixture was stirred at 155° C. under N₂ for 12 h. After cooled to room temperature, the reaction mixture was diluted with ethyl acetate (200 ml), and filtered through celite. The filtrate was washed with water (300 ml×2) and brine (100 ml), dried over Na₂SO₄, and concentrated under reduced pressure to give a residue which was purified through silica gel flash column chromatography (hexane/ethyl acetate=20/1) to afford ethyl 6-methoxybenzo[b]thiophene-5-carboxylate as a yellow solid (2.4 g, 49%). LC/MS (ES⁺) calcd for C₁₂H₁₂O₃S: 236.1; found: 237.1 [M+H]. ¹H NMR (400 MHz, CDCl₃): δ 8.24 (s, 1H), 7.41 (s, 1H), 7.30 (d, J=5.6 Hz, 1H), 7.28 (d, J=5.6 Hz, 1H), 4.40 (q, J=7.4 Hz, 2H), 3.96 (s, 3H), 1.41 (t, J=7.4 Hz, 3H).

Step 6: To a solution of ethyl 6-methoxybenzo[b]thiophene-5-carboxylate (3.3 g, 14.0 mmol) in dichloromethane (30 mL) was added dropwise a solution of BBr₃ (8.7 g, 34.9 mmol) in dichloromethane (20 mL) with dry ice-acetone bath. The resulting mixture was stirred at −70° C. under N₂ for 1 h. The reaction was quenched with methanol slowly at −10° C., and stirred at the same temperature for 30 min. The reaction mixture was partitioned between DCM and water; the organic phase was collected, and the aqueous phase was extracted with DCM. The combined organic phases was dried over Na₂SO₄ and concentrated under reduced pressure to give a residue which was purified through silica gel flash column chromatography (hexane/ethyl acetate=50/1) to afford ethyl 6-hydroxybenzo[b]thiophene-5-carboxylate as a white solid (2.3 g, 74%). LC/MS (ES⁺) calcd for C₁₁H₁₀O₃S: 222.0; found: 223.0 [M+H]. ¹H NMR (400 MHz, DMSO-d6): δ 10.59 (s, 1H), 8.37 (s, 1H), 7.61-7.58 (m, 2H), 7.46 (d, J=5.2 Hz, 1H), 4.41 (q, J=7.0 Hz, 2H), 1.38 (t, J=7.0 Hz, 3H).

Step 7: To a mixture of ethyl 6-hydroxybenzo[b]thiophene-5-carboxylate (2.0 g, 9 mmol) and 4-(2-chloroethyl)morpholine HCl salt (2.0 g, 10.8 mmol) in DMF (20 mL) was added Cs₂CO₃ (8.8 g, 27 mmol) at room temperature. The resulting mixture was heated to 85° C., and stirred for 3 hrs. The reaction mixture was cooled down to room temperature and filtered; the filtrate was diluted with ethyl acetate (80 ml), washed with water (100 ml×3) and brine (60 ml), dried over Na₂SO₄, and concentrated under reduced pressure to give a residue which was purified through silica gel flash column chromatography (DCM/MeOH=50/1) to afford ethyl 6-(2-morpholinoethoxy)benzo[b]thiophene-5-carboxylate as an off-white solid (2.79 g, 92%). LC/MS (ES⁺) calcd for C₁₇H₂₁NO₄S: 335.1; found: 336.4 [M+H]. ¹H NMR (400 MHz, CDCl₃): δ 8.22 (s, 1H), 7.41 (s, 1H), 7.31 (d, J=5.6 Hz, 1H), 7.28 (d, J=5.6 Hz, 1H), 4.38 (q, J=7.2 Hz, 2H), 4.23 (t, J=5.8 Hz, 2H), 3.76-3.71 (m, 4H), 2.89 (t, J=5.8 Hz, 2H), 2.65-2.60 (m, 4H), 1.40 (t, J=7.2 Hz, 3H).

Step 8: To a solution of ethyl 6-(2-morpholinoethoxy)benzo[b]thiophene-5-carboxylate (2.7 g, 8.3 mmol) in THF/MeOH/H₂O (4:1:1, 30 mL) was added LiOH.H₂O (2.1 g, 50 mmol) at room temperature. The resulting mixture was stirred at 60° C. for 3 h. THF and MeOH were removed under reduced pressure, and the residue was neutralized with HOAc to adjust the pH to 6. The resulting mixture was extracted with DCM-MeOH mixture (10:1 V/V); the combined organic phase was washed with brine, dried over Na₂SO₄, and concentrated under reduced pressure to give a residue which was triturated with diethyl ether to afford 6-[2-(morpholin-4-yl)ethoxy]-1-benzothiophene-5-carboxylic acid as a white solid (1.92 g, 75%). LC/MS (ES⁺) calcd for C₁₅H₁₇NO₄S: 307.1; found: 308.1 [M+H]. ¹H NMR (400 MHz, DMSO-d6): δ 8.22 (s, 1H), 7.85 (s, 1H), 7.67 (d, J=5.6 Hz, 1H), 7.45 (d, J=5.6 Hz, 1H), 4.57-4.52 (m, 2H), 3.89-3.84 (m, 4H), 3.62-3.57 (m, 2H), 3.37-3.26 (m, 4H).

### Intermediate 45: 6-[4-(morpholin-4-yl)butoxy]-1-benzothiophene-5-carboxylic Acid

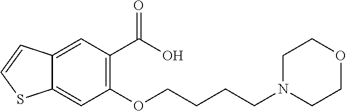 [SMILES: O=C(O)c1cc2ccsc2cc1OCCCCN1CCOCC1]

This compound can be prepared as described above for Intermediate 44: 6-[2-(morpholin-4-yl)ethoxy]-1-benzothiophene-5-carboxylic acid by substituting 4-(2-chloroethyl)morpholine with 4-(4-chlorobutyl)-morpholine (CAS No. 734495-59-1) step 7. LC/MS (ES⁺) calcd for C₁₇H₂₁NO₄S: 335.4; found: 3336.4 [M+H]. ¹H NMR (400 MHz, DMSO-d6): δ 8.37 (d, J=1.79 Hz, 1H), 7.57 (dd, J=7.55, 1.44 Hz, 1H), 7.49 (d, J=7.41 Hz, 1H), 7.42 (s, 1H), 4.03 (t, J=7.13 Hz, 2H), 3.59 (t, J=7.09 Hz, 4H), 2.60 (t, J=7.11 Hz, 2H), 2.47 (t, J=7.09 Hz, 4H), 1.84 (p, J=7.04 Hz, 2H), 1.58 (p, J=7.04 Hz, 2H).

### Intermediate 46: 6-[2-(morpholin-4-yl)ethoxy]-2H-1,3-benzodioxole-5-carboxylic Acid

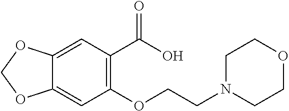 [SMILES: O=C(O)c1cc2c(cc1OCCN1CCOCC1)OCO2]

Step 1: A solution of benzo[d][1,3]dioxole-5-carboxylic acid (CAS No. 326-56-7, 15 g, 90.3 mmol) and concentrated sulfuric acid (0.1 mL) in methanol (200 mL) was stirred at 70° C. under nitrogen for 12 h. After completion of the reaction, the reaction mixture was cooled to room temperature, and concentrated under reduced pressure. The residue was diluted with water, neutralized with saturated aqueous Na₂CO₃ solution, and extracted with ethyl acetate. The combined organic layers were washed with brine, dried over sodium sulfate, and concentrated under reduced pressure to afford methyl benzo[d][1,3]dioxole-5-carboxylate as a white solid (16.0 g, 98%). LC/MS (ES⁺) calcd for C₉H₈O₄: 180.0; found: 181.0 [M+H]. ¹H NMR (400 MHz, DMSO-d6): δ 7.56 (dd, J=1.2, 8.0 Hz, 1H), 7.38 (d, J=0.8 Hz, 1H), 7.03 (d, J=8.4 Hz, 1H), 6.14 (s, 2H), 3.80 (s, 3H).

Step 2: To a stirred solution of methyl benzo[d][1,3]dioxole-5-carboxylate (16 g, 88.8 mmol) in acetic acid (100 mL) was added dropwise fuming nitric acid (111.5 g, 1.7 mol) at 20-25° C. under nitrogen. The resulting mixture was stirred at 20° C. for 30 min. After completion of the reaction, the reaction mixture was poured into ice-water. The precipitate was collected through filtration, washed with water, and dried to afford methyl 6-nitrobenzo[d][1,3]dioxole-5-carboxylate as a yellow solid (19.3 g, 97%). LC/MS (ES⁺) calcd for C₉H₇NO₆: 225.0; found: 226.1 [M+H]. ¹H NMR (400 MHz, DMSO-d6): δ 7.70 (s, 1H), 7.34 (s, 1H), 6.30 (s, 2H), 3.81 (s, 3H).

Step 3: A mixture of methyl 6-nitrobenzo[d][1,3]dioxole-5-carboxylate (19.3 g, 85.7 mmol) and Pd/C (10%, 1.9 g) in ethyl acetate/methanol (200 mL/100 mL) was stirred at 50° C. under hydrogen atmosphere (hydrogen balloon) for 12 h. After this time, the Pd/C was removed through celite and washed with methanol. The combined filtrate was concentrated under reduced pressure to afford methyl 6-aminobenzo[d][1,3]dioxole-5-carboxylate as an off-white solid (15 g, 90%). LC/MS (ES⁺) calcd for C₉H₉NO₄: 195.1; found: 196.1 [M+H]. ¹H NMR (400 MHz, DMSO-d6) δ 7.07 (s, 1H), 6.66 (s, 2H), 6.35 (s, 1H), 5.93 (s, 2H), 3.72 (s, 3H).

Step 4: To a mixture of methyl 6-aminobenzo[d][1,3]dioxole-5-carboxylate (11 g, 56.4 mmol) and concentrated sulfuric acid (12 mL) in water (60 mL) cooled with an ice-bath was added a mixture of sodium nitrite (3.9 g, 56.4 mmol) in water (25 mL). The resulting mixture was stirred at 0° C. for 15 minutes. After diluted with water (60 mL), the mixture was added into a boiling solution of cupric sulfate pentahydrate (56.4 g, 225.6 mmol) in water (130 mL). The resulting mixture was refluxed for 10 min, and then cooled down to room temperature with ice-bath. The reaction mixture was extracted with ethyl acetate (100 ml×2). The combined organic layer was washed with brine, dried over sodium sulfate, and concentrated under reduced pressure to give a crude product which was purified through silica gel flash column chromatography (hexane/ethyl acetate=50/1) to afford methyl 6-hydroxybenzo[d][1,3]dioxole-5-carboxylate as a white solid (7.5 g, 68%). LC/MS (ES⁺) calcd for C₉H₈O₅: 196.0; found: 197.0 [M+H]. ¹H NMR (400 MHz, DMSO-d6): δ 10.90 (s, 1H), 7.17 (s, 1H), 6.62 (s, 1H), 6.07 (s, 2H), 3.86 (s, 3H).

Step 5: To a mixture of methyl 6-hydroxybenzo[d][1,3]dioxole-5-carboxylate (3.0 g, 15.3 mmol) and cesium carbonate (10.0 g, 30.6 mmol) in DMF (50 mL) was added 1,2-dibromoethane (14.3 g, 76.5 mmol). The resulting mixture was stirred at 85° C. under nitrogen for 12 h. After completion of the reaction, the reaction mixture was cooled to room temperature and filtered. The filtrate was diluted with ethyl acetate (200 ml), washed with water (300 ml×2) and then brine (100 ml), dried over sodium sulfate, and concentrated under reduced pressure to give a crude product which was purified through silica gel flash column chromatography (hexane/ethyl acetate=20/1) to afford methyl 6-(2-bromoethoxy)benzo[d][1,3]dioxole-5-carboxylate as a white solid (1.5 g, 32%). LC/MS (ES⁺) calcd for C₁₁H₁₁BrO₅: 302.0; found: 304.9 [M+3]. ¹H NMR (400 MHz, DMSO-d6): δ 7.18 (s, 1H), 6.89 (s, 1H), 6.06 (s, 2H), 4.01 (t, J=6.0 Hz, 2H), 3.73 (s, 3H), 3.62 (t, J=6.8 Hz, 2H), 2.04-1.96 (m, 2H), 1.84-1.76 (m, 2H).

Step 6: A solution of methyl 6-(2-bromoethoxy)benzo[d][1,3]dioxole-5-carboxylate (1.5 g, 4.9 mmol) and morpholine (8.5 g, 98.0 mmol) in toluene (20 mL) was stirred at 100° C. 12 h. After completion of the reaction, the reaction mixture was cooled to room temperature, and concentrated under reduced pressure to give a residue which was purified through silica gel flash column chromatography (hexane/ethyl acetate=1/1) to afford methyl 6-(2-morpholinoethoxy)benzo[d][1,3]dioxole-5-carboxylate as a yellow oil (1.5 g, 98%). LC/MS (ES⁺) calcd for C₁₅H₁₉NO₆: 309.1; found: 310.3 [M+H]. ¹H NMR (400 MHz, DMSO-d6): δ 7.16 (s, 1H), 6.91 (s, 1H), 6.06 (s, 2H), 4.08 (t, J=5.6 Hz, 2H), 3.72 (s, 3H), 3.56 (t, J=4.4 Hz, 4H), 2.66 (t, J=5.6 Hz, 2H), 2.49-2.46 (m, 4H).

Step 7: To a stirred solution of methyl 6-(2-morpholinoethoxy)benzo[d][1,3]dioxole-5-carboxylate (1.5 g, 4.8 mmol) in methanol/water (1/1, 20 mL) was added LiOH.H₂O (1 g, 24.2 mmol). The resulting mixture was stirred at room temperature for 12 h. After completion of the reaction, the methanol was removed under reduced pressure, and the residue was acidified with diluted hydrochloric acid (1N) to pH 5-6. After concentration under reduced pressure, the crude product was purified through silica gel flash column chromatography (DCM/MeOH=10/1) to afford 6-(2-morpholinoethoxy)benzo[d][1,3]dioxole-5-carboxylic acid as an off-white solid (1.4 g, 98%). LC/MS (ES⁺) calcd for C₁₄H₁₇NO₆: 295.1; found: 296.3 [M+H]. H NMR (400 MHz, DMSO-d6): δ 12.40 (br, 1H), 7.20 (s, 1H), 6.98 (s, 1H), 6.07 (s, 2H), 4.48 (t, J=4.8 Hz, 2H), 3.89 (t, J=4.8 Hz, 4H), 3.55-3.47 (m, 6H).

### Intermediate 47: 6-[4-(morpholin-4-yl)butoxy]-2H-1,3-benzodioxole-5-carboxylic Acid

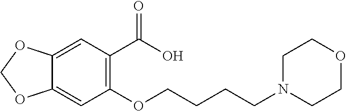 [SMILES: O=C(O)c1cc2c(cc1OCCCCN1CCOCC1)OCO2]

This compound can be prepared as described above for Intermediate 46: 6-[2-(morpholin-4-yl)ethoxy]-2H-1,3-benzodioxole-5-carboxylic acid by substituting 1,2-dibromoethane with 1,2-dibromobutane in step 5, LC/MS (ES⁺) calcd for C₁₆H₂₁NO₆: 323.3; found: 324.4 [M+H]. ¹H NMR (400 MHz, DMSO-d6): δ 7.56 (s, 1H), 6.71 (s, 1H), 6.06 (s, 1H), 4.03 (t, J=7.11 Hz, 1H), 3.59 (t, J=7.09 Hz, 2H), 2.60 (t, J=7.07 Hz, 1H), 2.46 (t, J=7.11 Hz, 2H), 1.82 (p, J=6.99 Hz, 1H), 1.58 (p, J=7.10 Hz, 1H).

### Intermediate 48: 3-[2-(morpholin-4-yl)ethoxy]-1-benzothiophene-2-carboxylic Acid

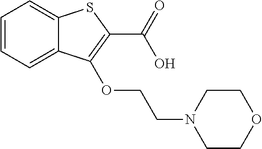 [SMILES: O=C(O)c1sc2ccccc2c1OCCN1CCOCC1]

This compound can be prepared as described above for Intermediate 46. LC/MS (ES⁺) calcd for C₁₅H₂₇NSO₄: 307.4; found: 308.4 [M+H]. ¹H NMR (400 MHz, DMSO-d6): δ 7.93 (1H, ddd, J=8.0, 1.5, 0.5 Hz), 7.72 (ddd, J=7.9, 1.5, 0.5 Hz), 7.69-7.83 (2H, 7.78 (ddd, J=8.0, 7.8, 1.5 Hz), 7.47 (1H, ddd, J=7.9, 7.8, 1.5 Hz), 4.18 (2H, t, J=5.9 Hz), 3.60 (4H, ddd, J=11.8, 10.2, 2.5 Hz), 2.91 (2H, t, J=5.9 Hz), 2.45 (4H, ddd, J=10.2, 9.7, 2.5 Hz).

### Intermediate 49 3-[2-(4,4-difluoropiperidin-1-yl)ethoxy]naphthalene-2-carboxylic Acid

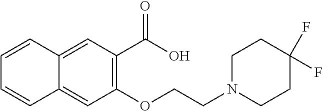 [SMILES: O=C(O)c1cc2ccccc2cc1OCCN1CCC(F)(F)CC1]

This compound can be prepared as described for Intermediate 37: 3-[3-(Morpholin-4-yl)ethoxy] naphthalene-2-carboxylic acid by substituting 3-morpholinopropan-1-ol step 1, with 4,4-Difluoro-1-piperidineethanol (CAS No. 276862-11-4). LC/MS (ES⁺) calcd for C₂₀H₂₃NF₂O₃: 306.4; found: 307.4 [M+H]. ¹H NMR (400 MHz, DMSO-d6): δ 8.58-8.54 (m, 1H), 7.93 (dt, J=7.29, 1.50 Hz, 1H), 7.78 (dt, J=7.40, 1.51 Hz, 1H), 7.57-7.50 (m, 2H), 7.49-7.42 (m, 1H), 4.08 (t, J=7.11 Hz, 2H), 3.00 (t, J=7.11 Hz, 2H), 2.67 (t, J=7.05 Hz, 4H), 2.19 (dtt, J=33.31, 20.85, 7.06 Hz, 4H).

### Intermediate 50: 3-[4-(4,4-difluoropiperidin-1-yl)butoxy]naphthalene-2-carboxylic Acid

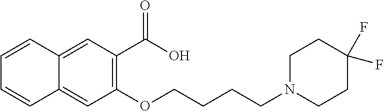 [SMILES: O=C(O)c1cc2ccccc2cc1OCCCCN1CCC(F)(F)CC1]

This compound can be prepared as described for Intermediate 37: 3-[3-(Morpholin-4-yl)ethoxy] naphthalene-2-carboxylic acid by substituting 3-morpholinopropan-1-ol step 1, with 4,4-Difluoro-1-piperidineethanol (CAS No. 276862-11-4). LC/MS (ES⁺) calcd for C₁₈H₁₉NF₂O₃: 363.4; found: 364.5 [M+H]. ¹H NMR (400 MHz, DMSO-d6): δ 8.58-8.54 (m, 1H), 7.93 (dt, J=7.29, 1.45 Hz, 1H), 7.76 (dt, J=7.61, 1.55 Hz, 1H), 7.59-7.52 (m, 2H), 7.46 (td, J=7.49, 1.57 Hz, 1H), 4.03 (t, J=7.06 Hz, 2H), 2.68 (t, J=7.14 Hz, 4H), 2.57 (t, J=7.11 Hz, 2H), 2.14 (tt, J=20.89, 7.00 Hz, 4H), 1.80 (p, J=7.09 Hz, 2H), 1.57 (p, J=7.03 Hz, 2H).

### Intermediate 51: 3-{2-[2-(methoxymethyl)morpholin-4-yl]ethoxy}naphthalene-2-carboxylic Acid

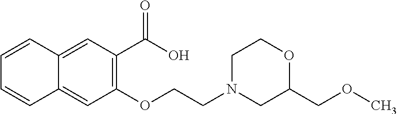 [SMILES: COCC1CN(CCOc2cc3ccccc3cc2C(=O)O)CCO1]

This compound can be prepared as described for Intermediate 37: 3-[3-(Morpholin-4-yl)ethoxy] naphthalene-2-carboxylic acid by substituting 3-morpholinopropan-1-ol step 1, with 2-(methoxymethyl)-4-morpholineethanol (CAS No. 2148484-23-3). LC/MS (ES⁺) calcd for C₁₉H₂₃NO₈: 345.9; found: 346.8 [M+H]. ¹H NMR (400 MHz, DMSO-d6): δ 8.46 (d, J=1.9 Hz, 1H), 7.99 (dt, J=8.3, 1.7 Hz, 1H), 7.82-7.74 (m, 2H), 7.70 (ddd, J=8.2, 6.7, 1.1 Hz, 1H), 7.57 (ddd, J=8.3, 7.1, 1.4 Hz, 1H), 4.25 (t, J=5.7 Hz, 2H), 3.99-3.87 (m, 2H), 3.75-3.65 (m, 2H), 3.58 (dd, J=11, 5, 4.4 Hz, 1H), 3.44 (s, 3H), 3.08 (dt, J=12.6, 5.7 Hz, 1H), 2.97 (dt, J=12.6, 5.6 Hz, 1H), 2.88 (ddd, J=12.5, 6.1, 3.4 Hz, 1H), 2.54 (dd, J=12.4, 3.8 Hz, 1H), 2.38 (ddd, J=12.4, 6.1, 3.4 Hz, 1H), 2.29 (dd, J=12.4, 3.8 Hz, 1H).

### Intermediate 52: 3-(2-{2-oxa-5-azabicyclo[2.2.2.2]octan-5-yl}ethoxy)naphthalene-2-carboxylic Acid

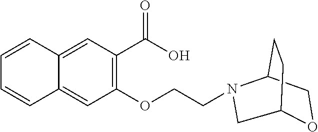 [SMILES: O=C(O)c1cc2ccccc2cc1OCCN1CC2CCC1CO2]

This compound can be prepared as described for Intermediate 37: 3-[3-(Morpholin-4-yl)ethoxy] naphthalene-2-carboxylic acid by substituting 3-morpholinopropan-1-ol step 1, with 3-Oxa-8-azabicyclo[3.2.1]octane-8-ethanol (CAS No. 1975173-15-9). LC/MS (ES⁺) calcd for C₁₉H₂₁NO₄: 327.4; found: 328.5 [M+H]. ¹H NMR (400 MHz, DMSO-d6): δ 8.46 (d, J=1.9 Hz, 1H), 7.99 (dt, J=8.1, 1.7 Hz, 1H), 7.79 (dt, J=7.2, 1.0 Hz, 1H), 7.78-7.74 (m, 1H), 7.70 (ddd, J=8.2, 6.7, 1.1 Hz, 1H), 7.57 (ddd, J=7.5, 6.8, 1.5 Hz, 1H), 4.20 (td, J=5.9, 2.5 Hz, 2H), 3.73 (dd, J=12.3, 3.7 Hz, 1H), 3.57 (tt, J=4.3, 2.6 Hz, 1H), 3.48 (dd, J=12.3, 3.7 Hz, 1H), 3.08 (dt, J=12.7, 5.9 Hz, 1H), 2.97 (dt, J=12.8, 6.0 Hz, 1H), 2.54 (dd, J=12.5, 2.6 Hz, 1H), 2.29 (dd, J=12.5, 2.6 Hz, 1H), 1.97 (tt, J=5.3, 3.7 Hz, 1H), 1.83-1.73 (m, 1H), 1.68-1.58 (m, 1H), 1.53 (ddt, J=12.6, 7.5, 5.1 Hz, 1H), 1.49-1.40 (m, 1H).

### Intermediate 53: 3-(2-{6-oxa-3-azabicyclo[3.1.1]heptan-3-yl}ethoxy)naphthalene-2-carboxylic Acid

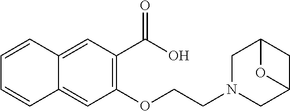 [SMILES: O=C(O)c1cc2ccccc2cc1OCCN1CC2CC(C1)O2]

This compound can be prepared as described for Intermediate 37: 3-[3-(Morpholin-4-yl)ethoxy] naphthalene-2-carboxylic acid by substituting 3-morpholinopropan-1-ol step 1, with 6-Oxa-3-azabicyclo[3.1.1]heptane-3-ethanol (CAS No. 1780777-65-2). LC/MS (ES⁺) calcd for C₁₈H₁₉NO₄: 313.4; found: 314.5 [M+H]. ¹H NMR (400 MHz, DMSO-d6): δ 8.46 (d, J=1.9 Hz, 1H), 7.99 (dt, J=8.0, 1.8 Hz, 1H), 7.79 (dt, J=7.4, 1.0 Hz, 1H), 7.78-7.74 (m, 1H), 7.73-7.67 (m, 1H), 7.57 (ddd, J=8.3, 6.8, 1.4 Hz, 1H), 4.68 (tt, J=4.6, 2.4 Hz, 2H), 4.25 (t, J=5.5 Hz, 2H), 3.08 (dt, J=12.6, 5.5 Hz, 1H), 2.97 (dt, J=12.6, 5.5 Hz, 1H), 2.73 (dt, J=12.4, 4.5 Hz, 1H), 2.54 (dd, J=12.4, 2.5 Hz, 2H), 2.48 (dt, J=12.5, 4.5 Hz, 1H), 2.29 (dd, J=12.4, 2.5 Hz, 2H).

### Intermediate 55: 3-{2-[(1S,4S)-2-oxa-5-azabicyclo[2.2.1]heptan-5-yl]ethoxy}naphthalene-2-carboxylic Acid

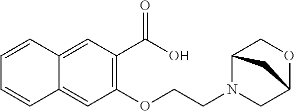 [SMILES: O=C(O)c1cc2ccccc2cc1OCCN1C[C@@H]2C[C@H]1CO2]

This compound can be prepared as described for Intermediate 37: 3-[3-(Morpholin-4-yl)ethoxy] naphthalene-2-carboxylic acid by substituting 3-morpholinopropan-1-ol step 1, with (1R,4R)-2-oxa-5-azabicyclo[2.2.1]heptane-5-ethanol, (CAS No. 743438-26-8). LC/MS (ES⁺) calcd for C₁₈H₁₉NO₄: 313.4; found: 314.5 [M+H]. ¹H NMR (400 MHz, DMSO-d6): δ 8.46 (d, J=2.0 Hz, 1H), 7.99 (dt, J=8.1, 1.8 Hz, 1H), 7.82-7.74 (m, 2H), 7.70 (ddd, J=8.1, 6.9, 1.3 Hz, 1H), 7.57 (ddd, J=8.0, 6.7, 1.2 Hz, 1H), 4.20 (td, J=6.0, 2.5 Hz, 2H), 3.94 (dd, J=12.3, 2.8 Hz, 1H), 3.72-3.66 (m, 2H), 3.08 (dt, J=12.7, 5.9 Hz, 1H), 2.97 (dt, J=12.8, 5.9 Hz, 1H), 2.63-2.55 (m, 2H), 2.35 (d, J=12.4 Hz, 1H), 1.98 (ddd, J=12.1, 4.0, 1.4 Hz, 1H), 1.73 (ddd, J=12.0, 3.9, 1.4 Hz, 1H).

### Intermediate 56: 6-[2-(morpholin-4-yl)ethoxy]-2,3-dihydro-1H-indene-5-carboxylic Acid

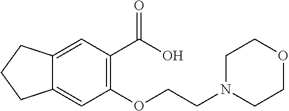 [SMILES: O=C(O)c1cc2c(cc1OCCN1CCOCC1)CCC2]

This compound can be prepared as described above for Intermediate 37: 3-[3-(Morpholin-4-yl)ethoxy] naphthalene-2-carboxylic acid by substituting by substituting methyl 3-hydroxy-2-naphthoate with 2,3-dihydro-6-hydroxy-1H-Indene-5-carboxylic acid (CAS No. 99058-98-7) in step 1. LC/MS (ES⁺) calcd for C₁₆H₂₁NO₄: 291.3.3; found: 292.4 [M+H]. ¹H NMR (400 MHz, DMSO-d6): δ 8.09 (t, J=1.0 Hz, 1H), 6.97 (t, J=1.0 Hz, 1H), 4.15 (t, J=6.0 Hz, 2H), 3.72 (dd, J=5.6, 3.8 Hz, 4H), 2.99-2.93 (m, 2H), 2.89-2.83 (m, 2H), 2.68 (t, J=5.9 Hz, 2H), 2.54-2.42 (m, 4H), 2.17-2.01 (m, 2H).

### Exemplary Preparation of Representative Compounds

Example 1: N-(6-methanesulfonyl-1,3-benzothiazol-2-yl)naphthalene-2-carboxamide

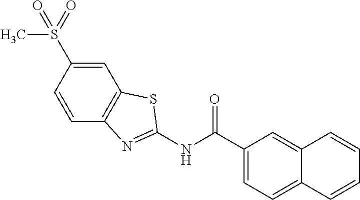 [SMILES: CS(=O)(=O)c1ccc2nc(NC(=O)c3ccc4ccccc4c3)sc2c1]

A mixture of 2-naphthalenecarboxylic acid (CAS No. 93-09-4, 300 mg, 1.7 mmol), TBTU (75 mg, 0.87 mmol), and DIEA (322 mg, 2.5 mmol) in acetonitrile (10 mL) was stirred at RT for 15 min, followed by addition of 6-(methylsulfonyl)-2-benzothiazolamine (Intermediate amine 2) (435 mg, 1.9 mmol) in one portion at RT. The resulting mixture was stirred at RT for 12 h. The reaction mixture was diluted with DCM (12 mL) and filtered. The filter cake was purified through column chromatography (eluent: DCM:MeOH from 50:1 to 20:1) to afford the desired product (519 mg, 75%) as a white solid. LC/MS (ES+): found: 383.5 [M+H]. ¹H NMR (400 MHz, DMSO-d6): δ 400 MHz, DMSO-d6) δ 8.45-8.39 (m, 2H), 8.08 (dt, J=7.54, 1.59 Hz, 1H), 8.00 (dd, J=7.77, 1.45 Hz, 1H), 7.96-7.89 (m, 4H), 7.61 (dtd, J=21.70, 7.45, 1.59 Hz, 2H), 4.33 (s, 1H), 3.22 (s, 2H).

The following compounds in Table 3 were prepared as described above for N-(6-methanesulfonyl-1,3-benzothiazol-2-yl)naphthalene-2-carboxamide (Example 1) with the appropriate carboxylic acid.

TABLE 3
Benzothiazolyl Compounds Prepared

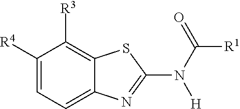

| | Amine | Acid | | | | |
|---|---|---|---|---|---|---|
| Ex. | (Int. | (Int. | | | | |
| No. | No.) | No.) | Name | R³ | R⁴ | R¹ |
| 1 | 1 | 29 | N-(6- methanesulfonyl- 1,3-benzothiazol- 2-yl)naphthalene- 2-carboxamide | H | 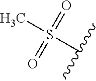 [SMILES: *S(C)(=O)=O] | 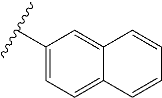 [SMILES: *c1ccc2ccccc2c1] |
| 2 | 2 | 29 | methyl 2- (naphthalene-2- amido)-1,3- benzothiazole-6- carboxylate | H | 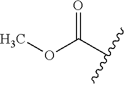 [SMILES: *C(=O)OC] | 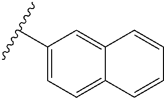 [SMILES: *c1ccc2ccccc2c1] |
| 3 | 23 | 29 | N-[6- (morpholine-4- sulfonyl)-1,3- benzothiazol-2- yl]naphthalene-2- carboxamide | H | 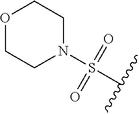 [SMILES: *S(=O)(=O)N1CCOCC1] | 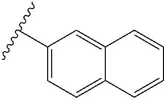 [SMILES: *c1ccc2ccccc2c1] |
| 4 | 3 | 29 | N-{6- [(difluoromethyl) sulfanyl]-1,3- benzothiazol-2- yl}naphthalene-2- carboxamide | H | 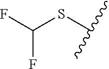 [SMILES: *SC(F)F] | 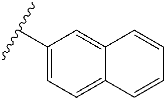 [SMILES: *c1ccc2ccccc2c1] |
| 5 | 4 | 29 | N-[6-(2,2,2- trifluoroethoxy)- 1,3-benzothiazol- 2-yl]naphthalene- 2-carboxamide | H | 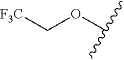 [SMILES: *OCC(F)(F)F] | 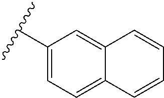 [SMILES: *c1ccc2ccccc2c1] |
| 6 | 5 | 29 | N-(6-benzamido- 1,3-benzothiazol- 2-yl)naphthalene- 2-carboxamide | H | 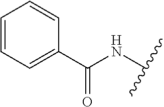 [SMILES: *NC(=O)c1ccccc1] | 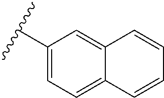 [SMILES: *c1ccc2ccccc2c1] |
| 7 | 82 | 29 | N-(6-methane sulfonamido-1,3- benzothiazol-2- yl)naphthalene-2- carboxamide | H | 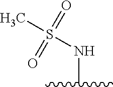 [SMILES: *NS(C)(=O)=O] | 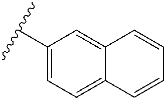 [SMILES: *c1ccc2ccccc2c1] |
| 8 | 8 | 29 | N-(6- cyclohexaneamido- 1,3- benzothiazol-2- yl)naphthalene-2- carboxamide | H | 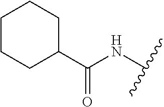 [SMILES: *NC(=O)C1CCCCC1] | 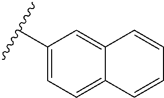 [SMILES: *c1ccc2ccccc2c1] |
| 9 | 3 | 29 | N-{6- [(trifluoromethyl) sulfanyl]-1,3- benzothiazol-2- yl}naphthalene-2- carboxamide | H | 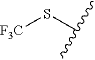 [SMILES: *SC(F)(F)F] | 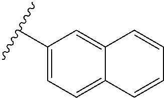 [SMILES: *c1ccc2ccccc2c1] |
| 10 | 10 | 29 | N-[6-(1H-1,3- benzodiazol-2- yl)-1,3- benzothiazol-2- yl]naphthalene-2- carboxamide | H | 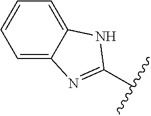 [SMILES: *c1nc2ccccc2[nH]1] | 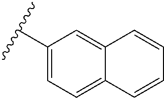 [SMILES: *c1ccc2ccccc2c1] |
| 11 | 11 | 26 | N-[7-(trifluoro methyl)-1,3- benzothiazol-2- yl]naphthalene-2- carboxamide | —CF₃ | H | 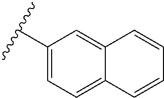 [SMILES: *c1ccc2ccccc2c1] |
| 12 | 12 | 29 | N-{6- [(cyclopropyl- carbamoyl)methyl]- 1,3-benzothiazol- 2-yl}naphthalene- 2-carboxamide | H | 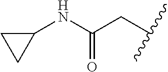 [SMILES: *CC(=O)NC1CC1] | 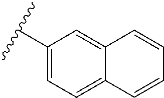 [SMILES: *c1ccc2ccccc2c1] |
| 13 | 13 | 29 | N-(2,6- dichlorophenyl)- 2-(naphthalene-2- amido)-1,3- benzothiazole-6- carboxaraide | H | 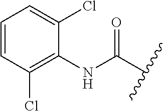 [SMILES: *C(=O)Nc1c(Cl)cccc1Cl] | 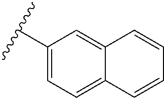 [SMILES: *c1ccc2ccccc2c1] |
| 14 | 14 | 29 | 2-(naphthalene-2- amido)-N- (pyridin-2-yl)- 1,3-benzo- thiazole-6- carboxamide | H | 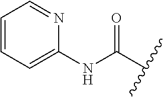 [SMILES: *C(=O)Nc1ccccn1] | 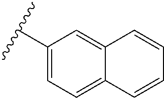 [SMILES: *c1ccc2ccccc2c1] |
| 15 | 13 | 30 | N-{6-[(2,6- dichlorophenyl) carbamoyl]-1,3- benzothiazol-2- yl}quinoline-6- carboxamide | H | 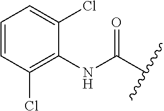 [SMILES: *C(=O)Nc1c(Cl)cccc1Cl] | 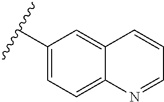 [SMILES: *c1ccc2ncccc2c1] |
| 16 | 23 | 38 | 3-[2-(morpholin- 4-yl)ethoxy]-N- [6-(morpholine-4- sulfonyl)-1,3- benzothiazol-2- yl]naphthalene-2- carboxamide | H | 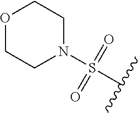 [SMILES: *S(=O)(=O)N1CCOCC1] | 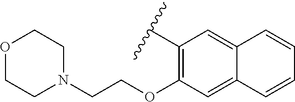 [SMILES: *c1cc2ccccc2cc1OCCN1CCOCC1] |
| 17 | 23 | 39 | 3-[4-(morpholin- 4-yl)butoxy]-N- [6-(morpholine-4- sulfonyl)-1,3- benzothiazol-2- yl]naphthalene-2- carboxamide | H | 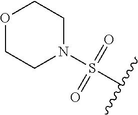 [SMILES: *S(=O)(=O)N1CCOCC1] | 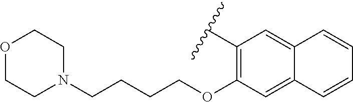 [SMILES: *c1cc2ccccc2cc1OCCCCN1CCOCC1] |
| 18 | 6 | 38 | N-(6-benzyl-1,3- benzothiazol-2- yl)-3-[2- (morpholin-4- yl)ethoxy] naphthalene-2- carboxamide | H | 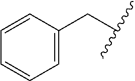 [SMILES: *Cc1ccccc1] | 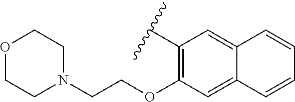 [SMILES: *c1cc2ccccc2cc1OCCN1CCOCC1] |
| 19 | 13 | 39 | N-(2,6- dichlorophenyl)- 2-{3-[4- (morpholin-4- yl)butoxy] naphthalene-2- amido}-1,3- | H | 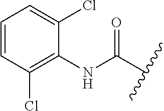 [SMILES: *C(=O)Nc1c(Cl)cccc1Cl] | 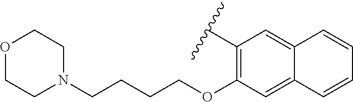 [SMILES: *c1cc2ccccc2cc1OCCCCN1CCOCC1] |
| | | | benzothiazole-6- | | | |
| | | | carboxamide | | | |
| 20 | 15 | 38 | 3-[2-(morpholin- 4-yl)ethoxy]-N- (6-nitro-1,3- benzothiazol-2- yl)naphthalene-2- carboxamide | H | 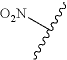 [SMILES: *[N+](=O)[O-]] | 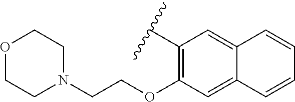 [SMILES: *c1cc2ccccc2cc1OCCN1CCOCC1] |
| 21 | 8 | 38 | N-(6- cyclohexaneamido- 1,3- benzothiazol-2- yl)-3-[2- (morpholin-4- yl)ethoxy] | H | 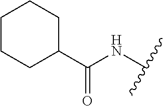 [SMILES: *NC(=O)C1CCCCC1] | 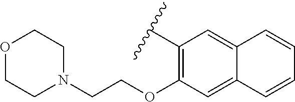 [SMILES: *c1cc2ccccc2cc1OCCN1CCOCC1] |
| | | | naphthalene-2- | | | |
| | | | carboxamide | | | |
| 22 | 8 | 39 | N-(6- cyclohexaneamido- 1,3- benzothiazol-2- yl)-3-[4- (morpholin-4- yl)butoxy] | H | 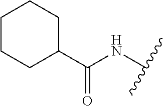 [SMILES: *NC(=O)C1CCCCC1] | 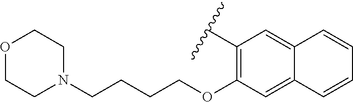 [SMILES: *c1cc2ccccc2cc1OCCCCN1CCOCC1] |
| | | | naphthalene-2- | | | |
| | | | carboxamide | | | |
| 23 | 15 | 39 | 3-[4-(morpholin- 4-yl)butoxy]-N- (6-nitro-1,3- benzothiazol-2- yl)naphthalene-2- carboxamide | H | 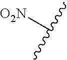 [SMILES: *[N+](=O)[O-]] | 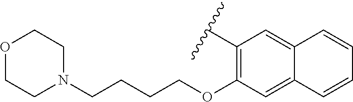 [SMILES: *c1cc2ccccc2cc1OCCCCN1CCOCC1] |
| 26 | 20 | 38 | 3-[2-(morpholin- 4-yl)ethoxy]-N- {7- [(phenylcarbamothioyl) amino]- 1,3-benzothiazol- 2-yl}naphthalene- 2-carboxamide | H | 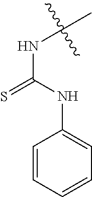 [SMILES: *NC(=S)Nc1ccccc1] | 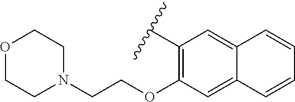 [SMILES: *c1cc2ccccc2cc1OCCN1CCOCC1] |
| 27 | 7 | 39 | N-(6- methanesulfonamido- 1,3- benzothiazol-2- yl)-3-[4- (morpholin-4- yl)butoxy] | H | 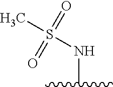 [SMILES: *NS(C)(=O)=O] | 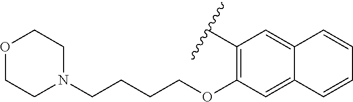 [SMILES: *c1cc2ccccc2cc1OCCCCN1CCOCC1] |
| | | | naphthalene-2- | | | |
| | | | carboxamide | | | |
| 28 | 7 | 38 | N-(6- methanesulfonamido- 1,3- benzothiazol-2- yl)-3-[2- (morpholin-4- yl)ethoxy] | H | 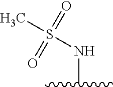 [SMILES: *NS(C)(=O)=O] | 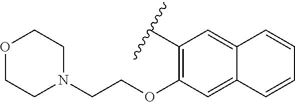 [SMILES: *c1cc2ccccc2cc1OCCN1CCOCC1] |
| | | | naphthalene-2- | | | |
| | | | carboxamide | | | |
| 29 | 22 | 38 | 3-[2-(morpholin- 4-yl)ethoxy]-N- [6-(pyrrolidine-1- sulfonyl)-1,3- benzothiazol-2- yl]naphthalene-2- carboxamide | H | 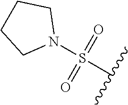 [SMILES: *S(=O)(=O)N1CCCC1] | 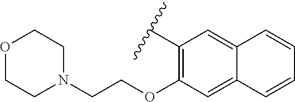 [SMILES: *c1cc2ccccc2cc1OCCN1CCOCC1] |
| 30 | 18′ | 38 | N-[6- (dimethylsulfamoyl)- 1,3- benzothiazol-2- yl]-3-[2- (morpholin-4- yl)ethoxy] | H | 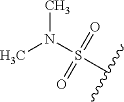 [SMILES: *S(=O)(=O)N(C)C] | 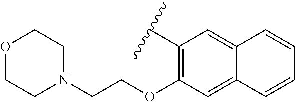 [SMILES: *c1cc2ccccc2cc1OCCN1CCOCC1] |
| | | | naphthalene-2- | | | |
| | | | carboxamide | | | |
| 31 | 22 | 39 | 3-[4-(morpholin- 4-yl)butoxy]-N- [6-(pyrrolidine-1- sulfonyl)-1,3- benzothiazol-2- yl]naphthalene-2- carboxamide | H | 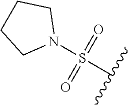 [SMILES: *S(=O)(=O)N1CCCC1] | 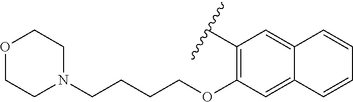 [SMILES: *c1cc2ccccc2cc1OCCCCN1CCOCC1] |
| 32 | 18 | 39 | 3-[4-(morpholin- 4-yl)butoxy]-N- (6-sulfamoyl-1,3- benzothiazol-2- yl)naphthalene- 2-carboxamide | H | 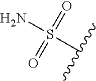 [SMILES: *S(N)(=O)=O] | 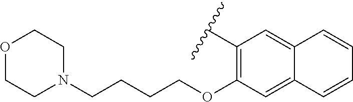 [SMILES: *c1cc2ccccc2cc1OCCCCN1CCOCC1] |
| 33 | 19 | 39 | 3-[4-(morpholin- 4-yl)butoxy]-N- {7- [(phenylcarbamothioyl) amino]- 1,3-benzothiazol- 2-yl}naphthalene- 2-carboxamide | H | 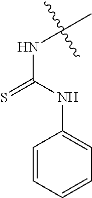 [SMILES: *NC(=S)Nc1ccccc1] | 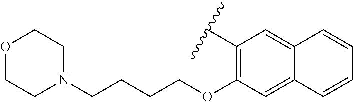 [SMILES: *c1cc2ccccc2cc1OCCCCN1CCOCC1] |
| 34 | 19 | 38 | 3-[2-(morpholin- 4-yl)ethoxy]-N- (7-{[(pyridin-2- yl)carbamothioyl] amino}-1,3- benzothiazol-2- yl)naphthalene-2- carboxamide | H | 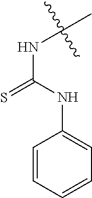 [SMILES: *NC(=S)Nc1ccccc1] | 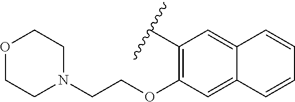 [SMILES: *c1cc2ccccc2cc1OCCN1CCOCC1] |
| 35 | 21 | 38 | N-[6-(azetidine-1- sulfonyl)-1,3- benzothiazol-2- yl]-3-[2- (morpholin-4- yl)ethoxy] | H | 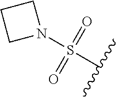 [SMILES: *S(=O)(=O)N1CCC1] | 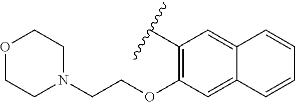 [SMILES: *c1cc2ccccc2cc1OCCN1CCOCC1] |
| | | | naphthalene-2- | | | |
| | | | carboxamide | | | |
| 36 | 21 | 39 | N-[6-(azetidine-1- sulfonyl)-1,3- benzothiazol-2- yl]-3-[4- (morpholin-4- yl)butoxy] | H | 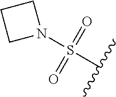 [SMILES: *S(=O)(=O)N1CCC1] | 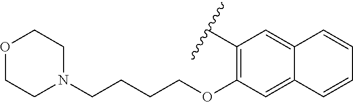 [SMILES: *c1cc2ccccc2cc1OCCCCN1CCOCC1] |
| | | | naphthalene-2- | | | |
| | | | carboxamide | | | |
| 37 | 24′ | 38 | 3-[2-(morpholin- 4-yl)ethoxy]-N- [6-(piperidine-1- sulfonyl)-1,3- benzothiazol-2- yl]naphthalene- 2-carboxamide | H | 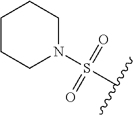 [SMILES: *S(=O)(=O)N1CCCCC1] | 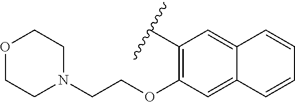 [SMILES: *c1cc2ccccc2cc1OCCN1CCOCC1] |
| 38 | 24′ | 39 | 3-[4-(morpholin- 4-yl)butoxy]-N- [6-(piperidine-1- sulfonyl)-1,3- benzothiazol-2-yl] naphthalene-2- carboxamide | H | 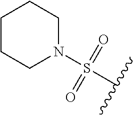 [SMILES: *S(=O)(=O)N1CCCCC1] | 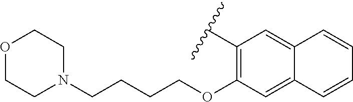 [SMILES: *c1cc2ccccc2cc1OCCCCN1CCOCC1] |
| 39 | 22 | 44 | 6-[2-(morpholin- 4-yl)ethoxy]-N- [6-(pyrrolidine-1- sulfonyl)-1,3- benzothiazol-2- yl]-1- benzothiophene- | H | 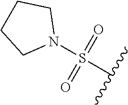 [SMILES: *S(=O)(=O)N1CCCC1] | 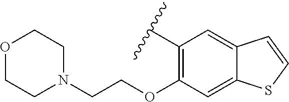 [SMILES: *c1cc2ccsc2cc1OCCN1CCOCC1] |
| | | | 5-carboxamide | | | |
| 41 | 22 | 43 | 3-[4-(morpholin- 4-yl)butoxy]-N- [6-(pyrrolidine-1- sulfonyl)-1,3- benzothiazol-2- yl]-[1,1′- biphenyl]-4- carboxamide | H | 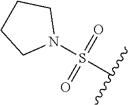 [SMILES: *S(=O)(=O)N1CCCC1] | 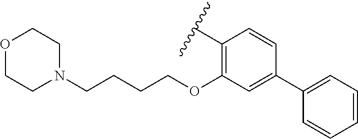 [SMILES: *c1ccc(-c2ccccc2)cc1OCCCCN1CCOCC1] |
| 42 | 22 | 45 | 6-[4-(morpholin- 4-yl)butoxy]-N- [6-(pyrrolidine-1- sulfonyl)-1,3- benzothiazol-2- yl]-1- benzothiophene- | H | 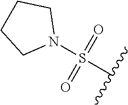 [SMILES: *S(=O)(=O)N1CCCC1] | 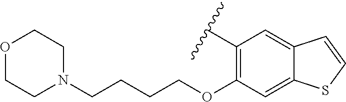 [SMILES: *c1cc2ccsc2cc1OCCCCN1CCOCC1] |
| | | | 5-carboxamide | | | |
| 43 | 22 | 38 | 6-[4-(morpholin- 4-yl)butoxy]-N- [6-(pyrrolidine-1- sulfonyl)-1,3- benzothiazol-2- yl]-2H-1,3- benzodioxole-5- | H | 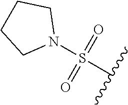 [SMILES: *S(=O)(=O)N1CCCC1] | 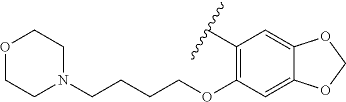 [SMILES: *c1cc2c(cc1OCCCCN1CCOCC1)OCO2] |
| | | | carboxamide | | | |
| 44 | 7 | 38 | N-(6- methanesulfonyl- 1,3-benzothiazol- 2-yl)-3-[2- (morpholin-4- yl)ethoxy] | H | 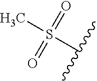 [SMILES: *S(C)(=O)=O] | 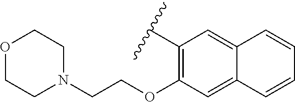 [SMILES: *c1cc2ccccc2cc1OCCN1CCOCC1] |
| | | | naphthalene-2- | | | |
| | | | carboxamide | | | |
| 45 | 7 | 39 | N-(6- methanesulfonyl- 1,3-benzothiazol- 2-yl)-3-[4- (morpholin-4- yl)butoxy] | H | 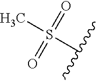 [SMILES: *S(C)(=O)=O] | 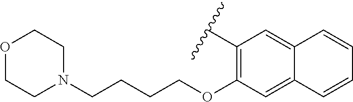 [SMILES: *c1cc2ccccc2cc1OCCCCN1CCOCC1] |
| | | | naphthalene-2- | | | |
| | | | carboxamide | | | |
| 46 | 21 | 29 | N-[6-(azetidine-1- sulfonyl)-1,3- benzothiazol-2- yl]naphthalene-2- carboxamide | H | 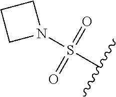 [SMILES: *S(=O)(=O)N1CCC1] | 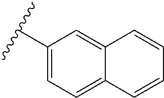 [SMILES: *c1ccc2ccccc2c1] |
| 47 | 22 | 43 | 3-[2-(morpholin- 4-yl)ethoxy]-N- [6-(pyrrolidine-1- sulfonyl)-1,3- benzothiazol-2- yl]-[1,1′- biphenyl]-4- carboxamide | H | 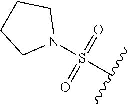 [SMILES: *S(=O)(=O)N1CCCC1] | 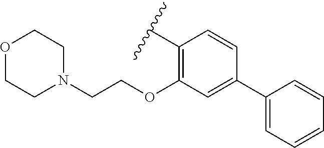 [SMILES: *c1ccc(-c2ccccc2)cc1OCCN1CCOCC1] |
| 48 | 4 | 38 | N-{6- [(difluoromethyl) sulfanyl]-1,3- benzothiazol-2- yl}-3-[2- (morpholin-4-yl) ethoxy]naphthalene- 2-carboxamide | H | 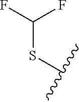 [SMILES: *SC(F)F] | 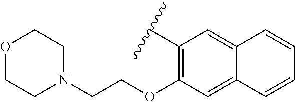 [SMILES: *c1cc2ccccc2cc1OCCN1CCOCC1] |
| 49 | 4 | 39 | N-{6- [(difluoromethyl) sulfanyl]-1,3- benzothiazol-2- yl}-3-[4- (morpholin-4-yl) butoxy]naphthalene- | H | 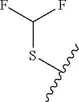 [SMILES: *SC(F)F] | 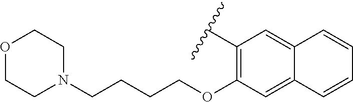 [SMILES: *c1cc2ccccc2cc1OCCCCN1CCOCC1] |
| | | | 2-carboxamide | | | |
| 50 | 21 | 31 | N-[6-(azetidine-1- sulfonyl)-1,3- benzothiazol-2- yl]-[1,1′- biphenyl]-4- carboxamide | H | 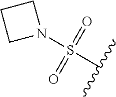 [SMILES: *S(=O)(=O)N1CCC1] | 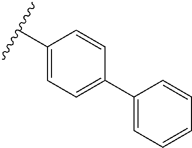 [SMILES: *c1ccc(-c2ccccc2)cc1] |
| 51 | 21 | 32 | N-[6-(azetidine-1- sulfonyl)-1,3- benzothiazol-2- yl]-1-methyl-1H- indole-2- carboxamide | H | 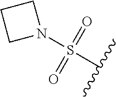 [SMILES: *S(=O)(=O)N1CCC1] | 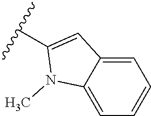 [SMILES: *c1cc2ccccc2n1C] |
| 52 | 22 | 32 | 1-methyl-N-[6- (pyrrolidine-1- sulfonyl)-1,3- benzothiazol-2- yl]-1H-indole-2- carboxamide | H | 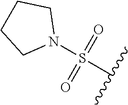 [SMILES: *S(=O)(=O)N1CCCC1] | 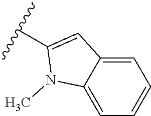 [SMILES: *c1cc2ccccc2n1C] |
| 55 | 24 | 39 | 3-[4-(morpholin- 4-yl)butoxy]-N- (6-{2-oxa-6- azaspiro[3.3]heptane- 6-sulfonyl}- 1,3-benzothiazol- 2-yl)naphthalene- 2-carboxamide | H | 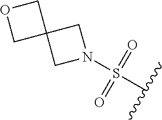 [SMILES: *S(=O)(=O)N1CC2(COC2)C1] | 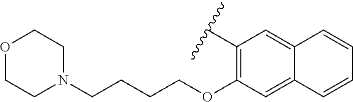 [SMILES: *c1cc2ccccc2cc1OCCCCN1CCOCC1] |
| 56 | 25 | 38 | N-{6-[(3- hydroxypyrrolidin- 1-yl)sulfonyl]- 1,3-benzothiazol- 2-yl}-3-[2- (morpholin-4- yl)ethoxy] naphthalene-2- carboxamide | H | 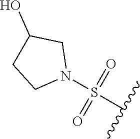 [SMILES: *S(=O)(=O)N1CCC(O)C1] | 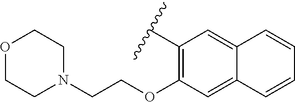 [SMILES: *c1cc2ccccc2cc1OCCN1CCOCC1] |
| 57 | 25 | 39 | N-{6-[(3- hydroxypyrrolidin- 1-yl)sulfonyl]- 1,3-benzothiazol- 2-yl}-3-[4- (morpholin-4- yl)butoxy] naphthalene-2- carboxamide | H | 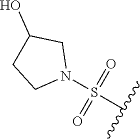 [SMILES: *S(=O)(=O)N1CCC(O)C1] | 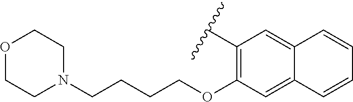 [SMILES: *c1cc2ccccc2cc1OCCCCN1CCOCC1] |
| 58 | 4 | 39 | 3-[4-(morpholin- 4-yl)butoxy]-N- [6-(2,2,2- trifluoroethoxy)- 1,3-benzothiazol- 2-yl]naphthalene- 2-carboxamide | H | 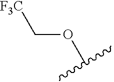 [SMILES: *OCC(F)(F)F] | 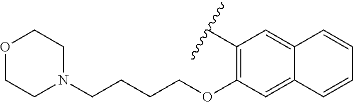 [SMILES: *c1cc2ccccc2cc1OCCCCN1CCOCC1] |
| 59 | 21 | 43 | N-[6-(azetidine-1- sulfonyl)-1,3- benzothiazol-2- yl]-3-[4- (morpholin-4- yl)butoxyl-[1,1′- biphenyl]-4- carboxamide | H | 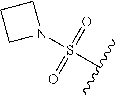 [SMILES: *S(=O)(=O)N1CCC1] | 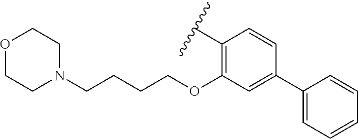 [SMILES: *c1ccc(-c2ccccc2)cc1OCCCCN1CCOCC1] |
| 60 | 21 | 42 | N-[6-(azetidine-1- sulfonyl)-1,3- benzothiazol-2- yl]-3-[2- (morpholin-4- yl)ethoxy]-[1,1′- biphenyl]-4- carboxamide | H | 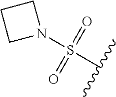 [SMILES: *S(=O)(=O)N1CCC1] | 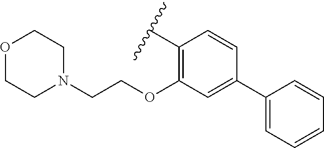 [SMILES: *c1ccc(-c2ccccc2)cc1OCCN1CCOCC1] |
| 61 | 21 | 47 | N-[6-(azetidine-1- sulfonyl)-1,3- benzothiazol-2- yl]-6-[2- (morpholin-4- yl)ethoxy]-2H- | H | 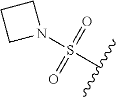 [SMILES: *S(=O)(=O)N1CCC1] | 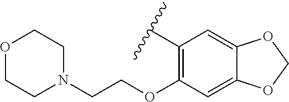 [SMILES: *c1cc2c(cc1OCCN1CCOCC1)OCO2] |
| | | | 1,3-bezodioxole- | | | |
| | | | 5-carboxamide | | | |
| 62 | 21 | 44 | N-[6-(azetidine-1- sulfonyl)-1,3- benzothiazol-2- yl]-6-[2- (morpholin-4- yl)ethoxy]-1- | H | 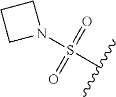 [SMILES: *S(=O)(=O)N1CCC1] | 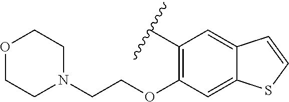 [SMILES: *c1cc2ccsc2cc1OCCN1CCOCC1] |
| | | | benzothiophene- | | | |
| | | | 5-carboxamide | | | |
| 63 | 21 | 45 | N-[6-(azetidine-1- sulfonyl)-1,3- benzothiazol-2- yl]-6-[4- (morpholin-4- yl)butoxy]-1- | H | 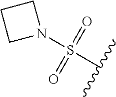 [SMILES: *S(=O)(=O)N1CCC1] | 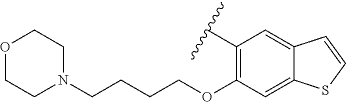 [SMILES: *c1cc2ccsc2cc1OCCCCN1CCOCC1] |
| | | | benzothiophene- | | | |
| | | | 5-carboxamide | | | |
| 64 | 21 | 46 | N-[6-(azetidine-1- sulfonyl)-1,3- benzothiazol-2- yl]-6-[4- (morpholin-4- yl)butoxy]-2H- | H | 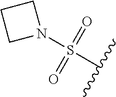 [SMILES: *S(=O)(=O)N1CCC1] | 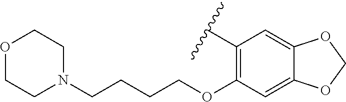 [SMILES: *c1cc2c(cc1OCCCCN1CCOCC1)OCO2] |
| | | | 1,3-benzodioxole- | | | |
| | | | 5-carboxamide | | | |
| 65 | 24 | 38 | 3-[2-(morpholin- 4-yl)ethoxy]-N- (6-{2-oxa-6- azaspiro[3.3]heptane- 6-sulfonyl}- 1,3-benzothiazol- 2-yl)naphthalene- 2-carboxamide | H | 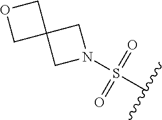 [SMILES: *S(=O)(=O)N1CC2(COC2)C1] | 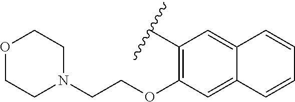 [SMILES: *c1cc2ccccc2cc1OCCN1CCOCC1] |
| 66 | 2 | 39 | methyl 2-{3-[4- (morpholin-4- yl)butoxy] naphthalene- 2-amido}-1,3- benzothiazole-6- carboxylate | H | 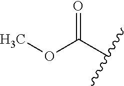 [SMILES: *C(=O)OC] | 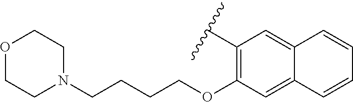 [SMILES: *c1cc2ccccc2cc1OCCCCN1CCOCC1] |
| 67 | 22 | 41 | 1-methyl-5-[2- (morpholin-4- yl)ethoxy]-N-[6- (pyrrolidine-1- sulfonyl)-1,3- benzothiazol-2- | H | 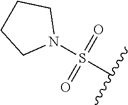 [SMILES: *S(=O)(=O)N1CCCC1] | 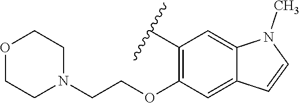 [SMILES: *c1cc2c(ccn2C)cc1OCCN1CCOCC1] |
| | | | yl]-1H-indole-6- | | | |
| | | | carboxamide | | | |
| 69 | 2 | 38 | methyl 2-{3-[2- (morpholin-4- yl)ethoxy] naphthalene- 2-amido}-1,3- benzothiazole-6- carboxylate | H | 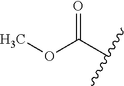 [SMILES: *C(=O)OC] | 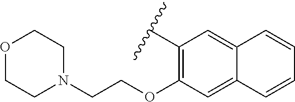 [SMILES: *c1cc2ccccc2cc1OCCN1CCOCC1] |
| 77 | 26 | 38 | N-(6-{[(3R)-3- hydroxypyrrolidin- 1-yl]sulfonyl}- 1,3-benzothiazol- 2-yl)-3-[2- (morpholin-4- yl)ethoxy] naphthalene-2- carboxamide | H | 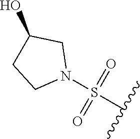 [SMILES: *S(=O)(=O)N1CC[C@@H](O)C1] | 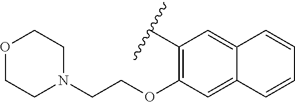 [SMILES: *c1cc2ccccc2cc1OCCN1CCOCC1] |
| 78 | 26 | 39 | N-(6-{[(3R)-3- hydroxypyrrolidin- 1-yl]sulfonyl}- 1,3-benzothiazol- 2-yl)-3-[4- (morpholin-4- yl)butoxy] naphthalene-2- carboxamide | H | 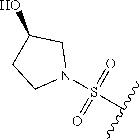 [SMILES: *S(=O)(=O)N1CC[C@@H](O)C1] | 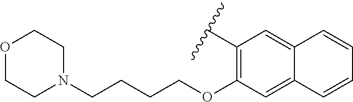 [SMILES: *c1cc2ccccc2cc1OCCCCN1CCOCC1] |
| 79 | 22 | 49 | 3-[2-(4,4- difluoropiperidin- 1-yl)ethoxy]-N- [6-(pyrrolidine-1- sulfonyl)-1,3- benzothiazol-2- yl]naphthalene-2- | H | 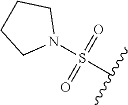 [SMILES: *S(=O)(=O)N1CCCC1] | 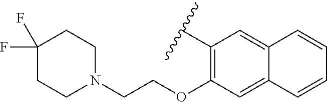 [SMILES: *c1cc2ccccc2cc1OCCN1CCC(F)(F)CC1] |
| | | | carboxamide | | | |
| 80 | 22 | 50 | 3-[4-(4,4- difluoropiperidin- 1-yl)butoxy]-N- [6-(pyrrolidine-1- sulfonyl)-1,3- benzothiazol-2- yl]naphthalene-2- | H | 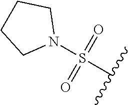 [SMILES: *S(=O)(=O)N1CCCC1] | 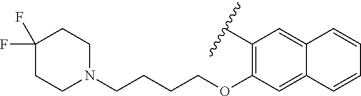 [SMILES: *c1cc2ccccc2cc1OCCCCN1CCC(F)(F)CC1] |
| | | | carboxamide | | | |
| 81 | 22 | 31 | N-[6-(pyrrolidine- 1-sulfonyl)-1- benzothiophen-2- yl]-[1,1′- biphenyl]-4- carboxamide | H | 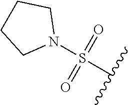 [SMILES: *S(=O)(=O)N1CCCC1] | 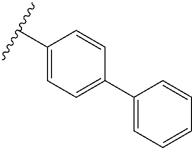 [SMILES: *c1ccc(-c2ccccc2)cc1] |
| 82 | 27 | 44 | N-(6-{[(3S)-3- hydroxypyrrolidin- 1-yl]sulfonyl}- 1,3-benzothiazol- 2-yl)-3-[2- (morpholin-4- yl)ethoxy] naphthalene-2- carboxamide | H | 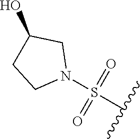 [SMILES: *S(=O)(=O)N1CC[C@@H](O)C1] | 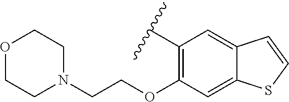 [SMILES: *c1cc2ccsc2cc1OCCN1CCOCC1] |
| 83 | 27 | 45 | N-(6-{[(3S)-3- hydroxypyrrolidin- 1-yl]sulfonyl}- 1,3-benzothiazol- 2-yl)-3-[4- (morpholin-4- yl)butoxy] naphthalene-2- carboxamide | H | 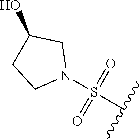 [SMILES: *S(=O)(=O)N1CC[C@@H](O)C1] | 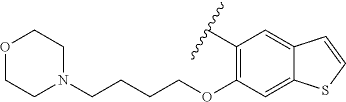 [SMILES: *c1cc2ccsc2cc1OCCCCN1CCOCC1] |
| 84 | 22 | 48 | 3-[2-(morpholin- 4-yl)ethoxy]-N- [6-(pyrrolidine-1- sulfonyl)-1,3- benzothiazol-2- yl]-1- benzothiophene- 2-carboxamide | H | 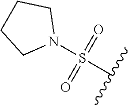 [SMILES: *S(=O)(=O)N1CCCC1] | 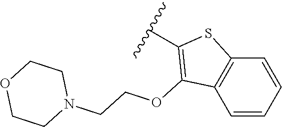 [SMILES: *c1sc2ccccc2c1OCCN1CCOCC1] |
| 24 | 17 | 38 | N-(6-cyano-1,3- benzothiazol-2- yl)-3-[2- (morpholin-4- yl)ethoxy] naphthalene-2- carboxamide | H | 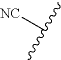 [SMILES: *C#N] | 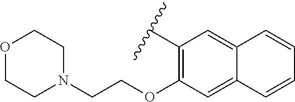 [SMILES: *c1cc2ccccc2cc1OCCN1CCOCC1] |
| 25 | 17 | 39 | N-(6-cyano-1,3- benzothiazol-2- yl)-3-[4- (morpholin-4- yl)butoxy] naphthalene-2- carboxamide | H | 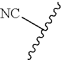 [SMILES: *C#N] | 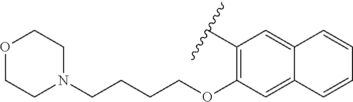 [SMILES: *c1cc2ccccc2cc1OCCCCN1CCOCC1] |
| 54 | 4 | 38 | 3-[2-(morpholin- 4-yl)ethoxy]-N- [6-(2,2,2- trifluoroethoxy)- 1,3-benzothiazol- 2-yl]naphthalene- 2-carboxamide | H | 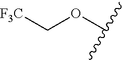 [SMILES: *OCC(F)(F)F] | 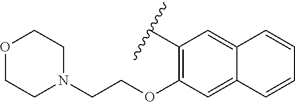 [SMILES: *c1cc2ccccc2cc1OCCN1CCOCC1] |
| 40 | 22 | 46 | 6-[2-(morpholin- 4-yl)ethoxy]-N- [6-(pyrrolidine-1- sulfonyl)-1,3- benzothiazol-2- yl]-2H-1,3- benzodioxole-5- | H | 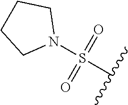 [SMILES: *S(=O)(=O)N1CCCC1] | 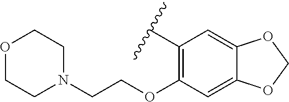 [SMILES: *c1cc2c(cc1OCCN1CCOCC1)OCO2] |
| | | | carboxamide | | | |
| 53 | 22 | 33 | 4-(pyridin-3-yl)- N-[6-(pyrrolidine- 1-sulfonyl)-1,3- benzothiazol-2- yl]benzamide | H | 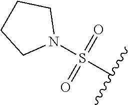 [SMILES: *S(=O)(=O)N1CCCC1] | 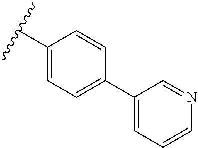 [SMILES: *c1ccc(-c2cccnc2)cc1] |
| 87 | 22 | 51 | 3-{2-[2- (methoxymethyl) morpholin-4- yl]ethoxy}-N-[6- (pyrrolidine-1- sulfonyl)-1,3- benzothiazol-2- yl]naphthalene-2- | H | 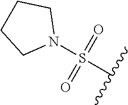 [SMILES: *S(=O)(=O)N1CCCC1] | 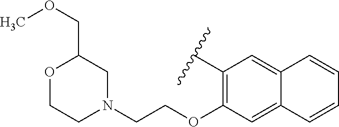 [SMILES: *c1cc2ccccc2cc1OCCN1CCOC(COC)C1] |
| | | | carboxamide | | | |
| 88 | 22 | 52 | 3-(2-{2-oxa-5- azabicyclo[2.2.2] octan-5- yl}ethoxy)-N-[6- (pyrrolidine-1- sulfonyl)-1,3- benzothiazol-2- | H | 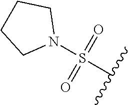 [SMILES: *S(=O)(=O)N1CCCC1] | 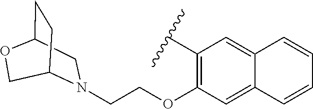 [SMILES: *c1cc2ccccc2cc1OCCN1CC2CCC1CO2] |
| | | | yl]naphthalene-2- | | | |
| | | | carboxamide | | | |
| 89 | 22 | 53 | 3-(2-{6-oxa-3- azabicyclo[3.1.1] heptan-3- yl}ethoxy)-N-[6- (pyrrolidine-1- sulfonyl)-1,3- benzothiazol-2- | H | 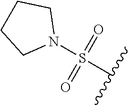 [SMILES: *S(=O)(=O)N1CCCC1] | 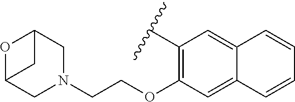 [SMILES: *c1cc2ccccc2cc1OCCN1CC2CC(C1)O2] |
| | | | yl]naphthalene-2- | | | |
| | | | carboxamide | | | |
| 90 | 22 | 55 | | H | 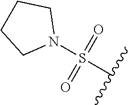 [SMILES: *S(=O)(=O)N1CCCC1] | 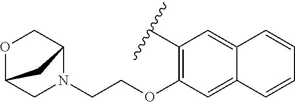 [SMILES: *c1cc2ccccc2cc1OCCN1C[C@H]2C[C@@H]1CO2] |
| 91 | 22 | 56 | 6-[2-(morpholin- 4-yl)ethoxy]-N- [6-(pyrrolidine-1- sulfonyl)-1,3- benzothiazol-2- yl]-2,3-dihydro- 1H-indene-5- | H | 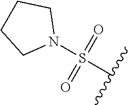 [SMILES: *S(=O)(=O)N1CCCC1] | 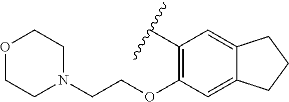 [SMILES: *c1cc2c(cc1OCCN1CCOCC1)CCC2] |
| | | | carboxamide | | | |

NMR and LCMS mass spectrometry data for the benzothiazolyl compounds of Table 3 are provided in Table 4 below.

TABLE 4

| Characterization of Benzothiazolyl Compound | | |
|---|---|---|
| | | LCMS |
| Ex. No. | ¹H NMR | (MH⁺) |
| 1 | ¹H NMR (400 MHz, DMSO-d₆) δ 8.55 (d, J = 1.52 Hz, 1H), 8.35 | 381.45 |
| | (t, J = 1.50 Hz, 1H), 8.12-8.04 (m, 1H), 7.98-7.89 (m, 5H), | |
| | 7.80 (dd, J = 7.89, 1.55 Hz, 1H), 7.65-7.56 (m, 3H), 3.20 (s, | |
| | 3H). | |
| 2 | ¹H NMR (400 MHz, DMSO-d₆) δ 8.39-8.33 (m, 3H), 8.14 (dd, J = | 362.40 |
| | 7.41, 1.55 Hz, 1H), 8.07 (ddd, J = 6.45, 3.58, 1.66 Hz, 1H), 7.98- | |
| | 7.88 (m, 3H), 7.83 (dd, J = 7.98, 1.43 Hz, 1H), 7.79 (d, J = 7.51 | |
| | Hz, 1H), 7.65-7.55 (m, 3H), 3.91 (s, 3H). | |
| 3 | ¹H NMR (400 MHz, DMSO-d₆) δ 8.35 (t, J = 1.47 Hz, 1H), 8.30 | 453.53 |
| | (d, J = 1.65 Hz, 1H), 8.10 (ddd, J = 5.37, 3.83, 1.60 Hz, 1H), 7.99- | |
| | 7.85 (m, 5H), 7.61 (dt, J = 5.76, 3.75 Hz, 2H), 3.68 (t, J = 7.11 | |
| | Hz, 4H), 2.96 (t, J = 7.09 Hz, 4H). | |
| 4 | ¹H NMR (400 MHz, DMSO-d₆) δ 8.35 (t, J = 1.42 Hz, 1H), 8.13- | 386.43 |
| | 8.05 (m, 1H), 7.98-7.89 (m, 3H), 7.82 (dd, J = 7.98, 1.46 Hz, | |
| | 1H), 7.72 (d, J = 7.49 Hz, 1H), 7.65-7.56 (m, 3H), 6.66 (dd, J = | |
| | 50 Hz 1H). | |
| 5 | ¹H NMR (400 MHz, DMSO-d₆) δ 8.35 (t, J = 1.48 Hz, 1H), 8.10 | 402.39 |
| | (ddd, J = 5.87, 2.85, 1.46 Hz, 1H), 7.94 (ddd, J = 7.18, 5.37, 1.98 | |
| | Hz, 2H), 7.83 (dd, J = 7.69, 1.42 Hz, 1H), 7.68 (d, J = 7.52 Hz, | |
| | 1H), 7.65-7.57 (m, 2H), 7.43 (d, J = 1.53 Hz, 1H), 7.00 (dd, J = | |
| | 7.51, 1.45 Hz, 1H), 4.78-4.63 (m, 2H). | |
| 6 | ¹H NMR (400 MHz, DMSO-d₆) δ 8.92 (s, 1H), 8.42 (t, J = 1.00 | 423.49 |
| | Hz, 1H), 8.35 (t, J = 1.53 Hz, 1H), 8.13-8.06 (m, 1H), 7.95 (ddd, | |
| | J = 7.43, 5.81, 1.64 Hz, 4H), 7.88 (dd, J = 7.84, 1.49 Hz, 1H), | |
| | 7.74 (d, J = 1.08 Hz, 2H), 7.61 (dd, J = 5.70, 3.31 Hz, 2H), 7.61- | |
| | 7.54 (m, 1H), 7.54-7.46 (m, 2H). | |
| 7 | ¹H NMR (400 MHz, DMSO-d₆) δ 9.29 (s, 1H), 8.35 (t, J = 1.50 | 397.47 |
| | Hz, 1H), 8.14-8.07 (m, 1H), 7.98-7.91 (m, 2H), 7.82 (dd, J = | |
| | 7.95, 1.51 Hz, 1H), 7.73 (d, J = 7.60 Hz, 1H), 7.65-7.57 (m, | |
| | 2H), 7.50 (d, J = 1.46 Hz, 1H), 7.14 (dd, J = 7.50, 1.46 Hz, 1H), | |
| | 2.95 (s, 2H). | |
| 8 | ¹H NMR (400 MHz, DMSO-d₆) δ 12.93 (br, 1H), 10.00 (s, 1H), | 429.54 |
| | 8.83 (s, 1H), 8.39 (s, 1H), 8.17-7.95 (m, 4H), 7.75-7.50 (m, 4H), | |
| | 2.41-2.31 (m, 1H), 1.86-1.73 (m, 4H), 1.51-1.48 (m, 2H), 1.31- | |
| | 1.21 (m, 4H). | |
| 9 | ¹H NMR (400 MHz, DMSO-d₆) δ 8.35 (t, J = 1.52 Hz, 1H), 8.15 | 404.43 |
| | (d, J = 1.45 Hz, 1H), 8.09 (ddd, J = 6.15, 3.18, 1.58 Hz, 1H), 7.98- | |
| | 7.91 (m, 2H), 7.84 (ddd, J = 15.26, 7.68, 1.47 Hz, 2H), 7.74 (d, | |
| | J = 7.54 Hz, 1H), 7.65-7.57 (m, 2H). | |
| 10 | ¹H NMR (400 MHz, DMSO-d₆) δ 8.35 (t, J = 1.55 Hz, 1H), 8.26 | 420.49 |
| | (d, J = 1.51 Hz, 1H), 8.10 (ddt, J = 7.51, 5.18, 2.54 Hz, 1H), 7.98- | |
| | 7.89 (m, 3H), 7.88 (dd, J = 7.73, 1.56 Hz, 1H), 7.72 (d, J = 7.49 | |
| | Hz, 1H), 7.64-7.57 (m, 3H), 7.58 (dd, J = 7.33, 1.79 Hz, 1H), | |
| | 7.19 (dtd, J = 21.97, 7.48, 1.65 Hz, 2H). | |
| 11 | ¹H NMR (400 MHz, DMSO-d₆) δ 8.35 (t, J = 1.56 Hz, 1H), 8.10- | 372.37 |
| | 8.04 (m, 1H), 7.93 (ddd, J = 7.94, 3.67, 1.43 Hz, 2H), 7.82 (dd, | |
| | J = 7.60, 1.56 Hz, 2H), 7.65-7.58 (m, 2H), 7.57 (t, J = 7.42 Hz, | |
| | 1H), 7.52 (dd, J = 7.51, 1.65 Hz, 1H). | |
| 12 | ¹H NMR (400 MHz, DMSO-d₆) δ 8.35 (t, J = 1.48 Hz, 1H), 8.14- | 401.48 |
| | 8.07 (m, 1H), 7.98-7.91 (m, 2H), 7.86 (dd, J = 7.94, 1.48 Hz, | |
| | 1H), 7.81 (d, J = 7.50 Hz, 1H), 7.74 (d, J = 1.44 Hz, 1H), 7.65- | |
| | 7.57 (m, 2H), 7.41-7.32 (m, 2H), 3.61 (s, 1H), 2.71 (dp, J = | |
| | 9.15, 6.99 Hz, 1H), 0.79-0.68 (m, 2H), 0.70-0.63 (m, 1H), 0.66- | |
| | 0.59 (m, 1H). | |
| 13 | ¹H NMR (400 MHz, DMSO-d₆) δ 9.90 (s, 1H), 8.35 (t, J = 1.48 | 492.37 |
| | Hz, 1H), 8.10-8.04 (m, 2H), 7.95-7.87 (m, 3H), 7.75 (dd, J = | |
| | 7.51, 1.47 Hz, 1H), 7.70 (d, J = 7.44 Hz, 1H), 7.66-7.57 (m, | |
| | 2H), 7.51 (d, J = 7.40 Hz, 2H), 7.38 (dd, J = 7.87, 6.95 Hz, 1H). | |
| 14 | ¹H NMR (400 MHz, DMSO-d6): δ 13.22 (s, 1H), 10.80 (s, 1H), | 424.48 |
| | 8.87 (s, 1H), 8.77 (s, 1H), 8.41 (dd, J = 4.8 Hz, 1H), 8.23 (d, J = | |
| | 8.0 Hz, 1H), 8.21-8.13 (m, 2H), 8.12-8.07 (m, 2H), 8.05 (d, J = | |
| | 8.0 Hz, 1H), 7.92-7.82 (m, 2H), 7.74-7.63 (m, 2H), 7.18 (dd, J = | |
| | 7.2 Hz, 5.6 Hz, 1H). | |
| 15 | ¹H NMR (400 MHz, DMSO-d₆) δ 9.56 (s, 1H), 8.81 (dd, J = 7.51, | 493.36 |
| | 1.46 Hz, 1H), 8.38 (dt, J = 7.53, 1.69 Hz, 1H), 8.34 (t, J = 1.41 | |
| | Hz, 1H), 8.09 (d, J = 1.46 Hz, tH), 8.03 (d, J = 7.51 Hz, 1H), 7.88 | |
| | (dd, J = 7.51, 1.46 Hz, 1H), 7.76 (dd, J = 7.51, 1.47 Hz, 1H), 7.71 | |
| | (d, J = 7.42 Hz, 1H), 7.67 (t, J = 7.48 Hz, 1H), 7.08-7.03 (m, | |
| | 1H), 7.03-6.98 (m, 2H), 2.27 (d, J = 0.71 Hz, 4H). | |
| 16 | ¹H NMR (400 MHz, DMSO-d₆) 12.75 (s, 1H), 10.32 (s, 1H), 8.58 | 583.69 |
| | (s, 1H), 8.34 (s, 1H), 8.02 (dd, 2H), 7.97 (d, 1H), 7.81 (d, 1H), | |
| | 7.78 (t, 1H), 7.48 (s, 1H), 7.48 (t, 1H), 4.28 (bs, 2H), 3.91 (dd, | |
| | 2H), 3.62 (bs, 6H), 3.16 (bs, 2H), 2.91 (bs, 6H), 1.90 (bs, 4H). | |
| 17 | ¹H NMR (400 MHz, DMSO-d₆) 12.24 (s, 1H), 8.52 (s, 1H), 8.03 | 6H.74 |
| | (d, 1H), 7.91 (bs, 2H), 7.62 (d, 1H), 7.59 (bs, 2H), 7.35 (t, tH), | |
| | 7.32-7.25 (m, 5H), 7.19 (bs, 1H), 4.46 (bs, 2H), 4.07 (s, 2H), 3.59 | |
| | (bs, 6H), 2.87 (bs, 2H), 2.50 (s, 2H). | |
| 18 | ¹H NMR (400 MHz, DMSO-d₆) δ 12.66 (s, 1H), 11.09 (s, 1H), | 524.65 |
| | 10.43 (s, 1H), 8.73 (s, 1H), 8.36 (s, 1H), 8.14 (d, 1H, J = 8.37 Hz), | |
| | 8.01 (d, 1H, J = 8.09 Hz), 7.92 (d, 1H, J = 8.45 Hz), 7.65-7.56 | |
| | (m, 4H), 7.50-7.40 (m, 2H), 4.32-4.24 (m, 2H), 3.89 (d, 2H, J = | |
| | 10.46 Hz), 3.79 (t, 2H, J = 11.73 Hz), 3.37 (d, 2H, J = 12.10 | |
| | Hz), 3.22-3.14 (m, 2H), 3.06-2.96 (m, 2H), 2.02-1.88 (m, | |
| | 4H). | |
| 19 | ¹H NMR (400 MHz, DMSO-d₆) δ 12.83 (s, tH), 10.40 (s, 1H), | 650.59 |
| | 8.72 (s, 1H), 8.43 (d, 1H, J = 67.82 Hz), 8.13 (d, 1H, J = 8.23 Hz), | |
| | 8.04 (d, 1H, J = 7.97 Hz), 7.91 (t, 2H, J = 8.97 Hz), 7.62 (d, 2H, | |
| | J = 8.14 Hz), 7.48 (t, 2H, J = 7.52 Hz), 7.46-7.39 (m, 2H), 4.54 | |
| | (m, 2H), 3.99-3.52 (m, 2H), 3.18 (s, 1H), 2.87 (s, 2H), 2.56 (s, 1H). | |
| 20 | ¹H NMR (400 MHz, CD₃OD) δ 8.35 (s, 1H), 8.08 (s, 1H), 7.96 | 478.52 |
| | (dd, J = 8.0, 16.4 Hz, 2H), 7.92 (d, J = 8.0 Hz, 1H), 7.65-7.59 (m, | |
| | 2H), 7.54-7.47 (m, 2H), 4.78-4.73 (m, 2H), 4.11-4.04 (m, 2H), | |
| | 4.01-3.92 (m, 2H), 3.83-3.78 (m, 2H), 3.77-3.71 (m, 2H), 3.40- | |
| | 3.33 (m, 2H). | |
| 21 | ¹H NMR (400 MHz, DMSO-d₆) δ 9.26 (s, 1H), 8.47 (d, J = 1.48 | 558.70 |
| | Hz, 1H), 7.96-7.89 (m, 2H), 7.85 (d, J = 7.51 Hz, 1H), 7.71 | |
| | (ddd, J = 7.51, 5.16, 1.56 Hz, 2H), 7.57 (d, J = 1.49 Hz, 1H), 7.52- | |
| | 7.44 (m, 2H), 4.03 (t, J = 7.05 Hz, 2H), 3.60 (t, J = 7.09 Hz, | |
| | 4H), 2.60 (t, J = 7.06 Hz, 2H), 2.47 (t, J = 7.09 Hz, 4H), 1.80 (p, | |
| | J = 7.11 Hz, 2H), 1.59 (p, J = 6.96 Hz, 2H). | |
| 22 | ¹H NMR (400 MHz, DMSO-d6) δ 12.33 (s, 1H), 10.02 (s, 1H), | 587.75 |
| | 8.41 (s, 1H), 8.33 (s, 1H), 7.99 (d, J = 7.2 Hz, 1H), 7.89 (d, J = 7.6 | |
| | Hz, 1H), 7.69 (d, J = 8.0 Hz, 1H), 7.65-7.50 (m, 3H), 7.49-7.40 | |
| | (m, 1H), 4.32-4.20 (m, 2H), 3.80-3.45 (m, 4H), 3.10-2.80 (m, 3H), | |
| | 2.43-2.30 (m, 3H), 1.95-1.60 (m, 9H), 1.52-1.37 (m, 2H), 1.36- | |
| | 1.15 (m, 4H). | |
| 23 | ¹H NMR (400 MHz, DMSO-d6) δ 11.24 (s, 1H), 9.61 (s, 2H), | 506.58 |
| | 8.94 (d, J = 7.2 Hz, 1H), 8.30 (s, 1H), 8.05-7.95 (m, 1H), 7.89 (d, | |
| | J = 8.0 Hz, 1H), 7.59 (t, J = 7.4 Hz, 1H), 7.53 (s, 1H), 7.48-7.41 | |
| | (m, 1H), 4.33-4.24 (m, 2H), 4.00-3.90 (m, 2H), 3.65-3.55 (m, 2H), | |
| | 3.54-3.45 (m, 2H), 3.33-3.24 (m, 2H), 3.16-3.04 (m, 2H), 1.99- | |
| | 1.87 (m, 4H). | |
| 26 | ¹H NMR (400 MHz, DMSO-d6) δ 12.35 (s, 1H), 9.83 (s, 1H), | 583.73 |
| | 8.33 (s, 1H), 7.99 (d, J = 8.0 Hz, 1H), 7.89 (d, J = 8.4 Hz, 1H), | |
| | 7.86 (d, J = 2.0 Hz, 1H), 7.73 (d, J = 8.4 Hz, 1H), 7.59 (t, J = 7.6 | |
| | Hz, 1H), 7.54 (s, 1H), 7.44 (t, J = 7.6 Hz, 1H), 7.32 (dd, J = 2.2, | |
| | 8.6 Hz, 1H), 4.24 (t, J = 6.0 Hz, 2H), 3.42-3.37 (m, 4H), 3.00 (s, | |
| | 3H), 2.29 (t, J = 7.0 Hz, 2H), 2.24-2.16 (m, 4H), 1.88-1.79 (m, | |
| | 2H), 1.69-1.59 (m, 2H). | |
| 27 | ¹H NMR (400 MHz, CDCl₃) δ 12.03 (br, 1H), 8.90 (s, 1H), 8.37 | 554.68 |
| | (d, J = 1.6 Hz, 1H), 7.95 (d, J = 8.0 Hz, 1H), 7.87 (dd, J = 1.6, 8.4 | |
| | Hz, 1H), 7.80 (d, J = 8.4 Hz, 1H), 7.77 (d, J = 8.0 Hz, 1H), 7.60 (t, | |
| | J = 7.6 Hz, 1H), 7.46 (t, J = 7.4 Hz, 1H), 7.32 (s, 1H), 4.47 (t, J = | |
| | 4.2 Hz, 2H), 3.82 (t, J = 4.4 Hz, 4H), 3.33-3.27 (m, 4H), 3.07-3.02 | |
| | (m, 2H), 2.75-2.67 (m, 4H), 1.81-1.75 (m, 4H). | |
| 28 | ¹H NMR (400 MHz, DMSO-d6) 12.62 (s, 1H), 11.38-11.26 (m, | 564.09 |
| | 1H), 9.87 (s, 1H), 8.28 (s, 1H), 8.00 (d, J = 8.4 Hz, 1H), 7.93 (d, | |
| | J = 8.4 Hz, 1H), 7.88 (d, J = 2.0 Hz, 1H), 7.74 (d, J = 8.4 Hz, 1H), | |
| | 7.61 (t, J = 7.6 Hz, 1H), 7.60 (s, 1H), 7.48 (t, J = 7.4 Hz, 1H), 7.32 | |
| | (dd, J = 2.0, 8.4 Hz, 1H), 4.69-4.62 (m, 2H), 3.98-3.78 (m, 6H), | |
| | 3.67-3.58 (m, 4H), 3.02 (s, 3H). | |
| 29 | ¹H NMR (400 MHz, CDCl₃) δ 12.02 (s, 1H), 8.91 (s, 1H), 8.33 (s, | 566.69 |
| | 1H), 7.96 (d, J = 8.0 Hz, 1H), 7.83-7.81 (m, 2H), 7.78 (d, J = 8.0 | |
| | Hz, 1H), 7.60 (t, J = 8.0 Hz, 1H), 7.46 (t, J = 8.0 Hz, 1H), 7.32 (s, | |
| | 1H), 4.48 (t, J = 5.2 Hz, 2H), 3.82 (t, J = 4.6 Hz, 4H), 3.04 (t, J = | |
| | 5.0 Hz, 2H), 2.77 (s, 6H), 2.74-2.69 (m, 4H). | |
| 30 | ¹H NMR (400 MHz, DMSO-d6) δ 12.61 (br, 1H), 8.62 (d, J = 1.6 | 540.65 |
| | Hz, 1H), 8.33 (s, 1H), 8.00 (d, J = 8.4 Hz, 1H), 7.94 (d, J = 8.4 | |
| | Hz, 1H), 7.90 (d, J = 8.4 Hz, 1H), 7.86 (dd, J = 1.6, 8.8 Hz, 1H), | |
| | 7.60 (t, J = 7.4 Hz, 1H), 7.55 (s, 1H), 7.45 (t, J = 7.4 Hz, 1H), 4.25 | |
| | (t, J = 5.8 Hz, 2H), 3.60-3.22 (m, 6H), 3.22-3.16 (m, 4H), 2.50- | |
| | 2.00 (m, 4H), 1.90-1.80 (m, 2H), 1.76-1.58 (m, 6H). | |
| 31 | ¹H NMR (400 MHz, DMSO-d₆): δ 14.12 (s, 1H), 12.52 (br, 1H), | 594.75 |
| | 11.21 (s, 1H), 8.37 (dd, J = 5.2 Hz, 1.2 Hz, 1H), 8.32 (s, 1H), 8.00 | |
| | (d, J = 8.0 Hz, 1H), 7.89-7.94 (m, 2H), 7.82 (d, J = 7.6 Hz, 1H), | |
| | 7.71 (d, J = 8.0 Hz, 1H), 7.59 (t, J = 7.6 Hz, 1H), 7.51-7.55 (m, | |
| | 2H), 7.45 (t, J = 7.6 Hz, 1H), 7.33 (d, J = 8.4 Hz, 1H), 7.19 (dd, | |
| | J = 6.8 Hz, 6.0 Hz, 1H), 4.25 (t, J = 5.6 Hz, 2H), 3.45 (br, 4H), 3.35 | |
| | (br, 2H), 2.25 (br, 4H), 1.81-1.90 (m, 2H), 1.69 (br, 2H). | |
| 32 | ¹H NMR (400 MHz, DMSO-d6) δ 12.64 (s, 1H), 10.63 (s, 1H), | 541.65 |
| | 8.56 (s, 1H), 8.34 (s, 1H), 8.00 (d, J = 8.4 Hz, 1H), 7.92 (s, 2H), | |
| | 7.91 (d, J = 8.4 Hz, 1H), 7.60 (t, J = 7.6 Hz, 1H), 7.56 (s, 1H), | |
| | 7.46 (t, J = 7.6 Hz, 1H), 7.40 (s, 2H), 4.31-4.24 (m, 2H), 3.96-3.80 | |
| | (m, 2H), 3.77-3.67 (m, 2H), 3.45-3.42 (m, 2H), 3.22-2.92 (m, 4H), | |
| | 1.94-1.85 (m, 4H). | |
| 33 | ¹H NMR (400 MHz, DMSO-d6) δ 12.43 (br, 1H), 10.09 (s, 1H), | 612.78 |
| | 9.99 (s, 1H), 8.33 (s, 1H), 8.00 (d, J = 8.4 Hz, 1H), 7.89 (d, J = 8.0 | |
| | Hz, 1H), 7.66 (d, J = 8.0 Hz, 1H), 7.59 (t, J = 7.4 Hz, 1H), 7.56- | |
| | 7.51 (m, 3H), 7.50-7.41 (m, 2H), 7.39-7.31 (m, 3H), 7.15 (t, J = | |
| | 7.4 Hz, 1H), 4.28-4.21 (m, 2H), 3.55-3.40 (m, 4H), 3.37-3.32 (m, | |
| | 2H), 2.44-2.15 (m, 4H), 1.90-1.80 (m, 2H), 1.76-1.64 (m, 2H). | |
| 34 | ¹H NMR (400 MHz, DMSO-d6) δ 12.96 (s, 1H), 11.25 (s, 1H), | 584.71 |
| | 8.66 (d, 1H, J = 1.86 Hz), 8.32 (s, 1H), 8.02 (dd, 1H, J = 8.35, | |
| | 5.89 Hz), 7.95 (d, 1H, J = 8.27 Hz), 7.86 (dd, 1H, J = 8.51, 1.91 | |
| | Hz), 7.66-7.60 (m, 2H), 7.49 (t, 1H, J = 7.52 Hz), 4.66 (t, 2H, | |
| | J = 4.53 Hz), 3.96 (d, 2H, J = 12.92 Hz), 3.85 (t, 2H, J = 12.10 Hz), | |
| | 3.71 (t, 4H, J = 7.63 Hz), 3.64 (d, 4H, J = 9.93 Hz), 3.17 (d, 2H, | |
| | J = 11.15 Hz), 1.99 (p, 2H, J = 7.48 Hz). | |
| 35 | ¹H NMR (400 MHz, DMSO-d6) δ 12.79 (s, 1H), 10.42 (s, 1H), | 553.66 |
| | 8.65 (d, 1H, J = 1.84 Hz), 8.34 (s, 1H), 8.01 (dd, 2H, J = 8.37, | |
| | 2.71 Hz), 7.91 (d, 1H, J = 8.26 Hz), 7.86 (dd, 1H, J = 8.51, 1.92 | |
| | Hz), 7.63-7.58 (m, 1H), 7.56 (s, 1H), 7.46 (t, 1H, J = 7.58 Hz), | |
| | 4.27 (d, 2H, J = 6.06 Hz), 3.88 (d, 2H, J = 12.86 Hz), 3.69 (q, 6H, | |
| | J = 12.82, 10.14 Hz), 3.17 (s, 2H), 2.99 (d, 2H, J = 11.43 Hz), | |
| | 1.99 (q, 2H, J = 7.74 Hz), 1.89 (bs, 4H). | |
| 36 | ¹H NMR (400 MHz, DMSO-d6) δ 12.85 (br, 1H), 8.80 (s. 1H), | 581.72 |
| | 8.16-8.12 (m, 1H), 8.08 (t, J = 7.6 Hz, 2H), 8.03 (d, J = 8.4 Hz, | |
| | 1H), 8.00 (s, 1H), 7.82 (s, 1H), 7.71-7.61 (m, 2H), 2.72 (s, 3H). | |
| 37 | ¹H NMR (400 MHz, DMSO-d6) δ 12.33 (s, 1H), 10.33 (br, 1H), | 581.72 |
| | 10.02 (s, 1H), 8.42 (s, 1H), 8.33 (s, 1H), 8.00 (d, J = 8.0 Hz, 1H), | |
| | 7.90 (d, J = 8.0 Hz, 1H), 7.69 (d, J = 8.4 Hz, 1H), 7.64-7.50 (m, | |
| | 3H), 7.47-7.41 (m, 1H), 4.32-4.20 (m, 2H), 3.92-3.80 (m, 2H), | |
| | 3.68 (t, J = 12.0 Hz, 2H), 3.41-3.32 (m, 2H), 3.22-3.14 (m, 2H), | |
| | 3.09-2.94 (m, 2H), 2.00-1.86 (m, 4H), 1.86-1.72 (m, 4H), 1.70- | |
| | 1.63 (m, 1H), 1.50-1.38 (m, 2H), 1.36-1.16 (m, 4H). | |
| 38 | ¹H NMR (400 MHz, CD₃OD) δ 8.39 (s, 1H), 8.26 (s, 1H), 8.01 (d, | 609.77 |
| | J = 8.0 Hz, 1H), 7.94 (d, J = 8.0 Hz, 1H), 7.86 (d, J = 8.4 Hz, 1H), | |
| | 7.72 (d, J = 8.4 Hz, 1H), 7.69-7.59 (m, 2H), 7.54 (t, J = 7.6 Hz, | |
| | 1H), 7.15 (t, J = 56.4 Hz, 1H), 4.75 (br, 2H), 4.13 (d, J = 12.8 Hz, | |
| | 2H), 3.95 (t, J = 12.0 Hz, 2H), 3.88-3.69 (m, 4H), 3.42-3.38 (m, | |
| | 2H). | |
| 39 | ¹H NMR (400 MHz, DMSO-d₆) δ 8.62 (s, 1H), 8.25 (s, 1H), 7.93 | 609.77 |
| | (d, .1 = 8.8 Hz, 1H), 7.90-7.83 (m, 2H), 7.68 (d, J = 5.2 Hz, 1H), | |
| | 7.49 (d, J = 5.6 Hz, 1H), 4.23 (t, J = 6.0 Hz, 2H), 3.46-3.35 (m, | |
| | 4H), 3.28-3.24 (m, 2H), 3.18 (t, J = 6.0 Hz, 4H), 2.30-2.10 (m, | |
| | 4H), 1.88-1.76 (m, 2H), 1.72-1.56 (m, 6H). | |
| 41 | ¹H NMR (400 MHz, DMSO-d6) δ 12.76 (s, 1H), 10.99 (br, 1H), | 621.78 |
| | 8.64 (s, 1H), 8.20 (s, 1H), 7.97-7.92 (m, 2H), 7,90-7.83 (m, 1H), | |
| | 7.73 (d, J = 5.6 Hz, 1H), 7.51 (d, J = 5.2 Hz, 1H), 4.61 (br, 2H), | |
| | 4.03-3.90 (m, 2H), 3.83-3.76 (m, 2H), 3.70-3.55 (m, 4H), 3.25- | |
| | 3.10 (m, 6H), 1.70-1.58 (m, 4H). | |
| 42 | ¹H NMR (400 MHz, DMSO-d6) δ 13.26 (s, 1H), 8.62 (d, J = 1.2 | 601.77 |
| | Hz, 1H), 8.28 (d, J = 8.4 Hz, 2H), 8.02 (d, J = 8.4 Hz, 1H), 7.91 | |
| | (d, J = 8.4 Hz, 2H), 7.86 (dd, J = 8.4 Hz, 1.2 Hz, 1H), 7.81-7.79 | |
| | (m, 2H), 7.55-7.51 (m, 2H), 7.47-7.43 (m, 1H), 3.71 (t, J = 7.6 Hz, | |
| | 4H), 2.02-1.94 (m, 2H). | |
| 43 | ¹H NMR (400 MHz, DMSO-d6) δ 12.40 (s, 1H), 11.00 (br, 1H), | 561.67 |
| | 8.60 (d, J = 1.6 Hz, 1H), 7.91 (d, J = 8.4 Hz, 1H), 7.84 (dd, J = 8.4 | |
| | Hz, 1.6 Hz, 1H), 7.26 (s, 2H), 7.08 (s, 1H), 6.14 (s, 2H), 4.53 (br, | |
| | 2H), 4.00-3.90 (m, 2H), 3.83-3.76 (m, 2H), 3.66-3.54 (m, 4H), | |
| | 3.25-3.10 (m, 6H), 1.70-1.58 (m, 4H). | |
| 44 | ¹H NMR (400 MHz, DMSO-d6) δ 12.10 (s, 1H), 8.63 (d, J = 1.6 | 512.61 |
| | Hz, 1H), 8.43 (s, 1H), 8.23 (d, J = 8.8 Hz, 1H), 8.03 (dd, J = 8.4, | |
| | 1.6 Hz, 1H), 7.96-7.91 (m, 2H), 7.70 (dd, J = 8.4, 1.6 Hz, 1H), | |
| | 7.66 (d, J = 5.2 Hz, 1H), 7.34 (s, 1H), 3.19-3.16 (m, 4H), 1.67- | |
| | 1.59 (m, 4H). | |
| 45 | ¹H NMR (400 MHz, CDCl₃) δ 11.70 (s, 1H), 8.91 (s, 1H), 8.48 (d, | 540.67 |
| | J = 1.6 Hz, 1H), 7.98 (dd, J = 8.4 Hz, 1.6 Hz, 1H), 7.95 (d, J = 8.0 | |
| | Hz, 1H), 7.91 (d, J = 8.8 Hz, 1H), 7.77 (d, J = 8.4 Hz, 1H), 7.60 (t, | |
| | J = 7.6 Hz, 1H), 7.46 (t, J = 7.6 Hz, 1H), 7.33 (s, 1H), 4.43 (t, J = | |
| | 6.0 Hz, 2H), 3.72 (br, 4H), 3.13 (s, 1H), 2.54-2.53 (br, 6H), 2.22- | |
| | 1.92 (br, 2H). | |
| 46 | ¹H NMR (400 MHz, DMSO-d6) δ 13.37 (s, 1H), 8.90 (s, 1H), | 424.51 |
| | 8.65 (s, 1H), 8.19 (dd, J = 8.8 Hz, 1.6 Hz, 1H), 8.13-8.10 (m, 2H), | |
| | 8.07-8.04 (m, 2H), 7.87 (dd, J = 8.4 Hz, 1.6 Hz, 1H), 7.74-7.66 (m, | |
| | 2H), 3.71 (t, J = 7.6 Hz, 4H), 2.03-1.95 (m, 2H). | |
| 47 | ¹H NMR (400 MHz, CDCl₃) δ 11.53 (br, 1H), 8.38 (d, J = 8.4 Hz, | 601.77 |
| | 1H), 8.36 (s, 1H), 7.92-7.84 (m, 2H), 7.67-7.60 (m, 2H), 7.53-7.47 | |
| | (m, 2H), 7.47-7.43 (m, 1H), 7.43-7.38 (m, 1H), 7.26 (s, 1H), 4.42 | |
| | (t, J = 6.0 Hz, 2H), 3.69 (t, J = 4.8 Hz, 4H), 3.34-3.25 (m, 4H), | |
| | 2.55 (t, J = 7.2 Hz, 2H), 2.52-2.45 (m, 4H), 2.18-2.11 (m, 2H), | |
| | 1.93-1.84 (m, 2H), 1.81-1.73 (m, 4H). | |
| 48 | ¹H NMR (400 MHz, CD₃OD) δ 8.48 (s, 1H), 8.20 (s, 1H), 7.94 (d, | 516.59 |
| | J = 8.0 Hz, 1H), 7.85 (d, J = 8.0 Hz, 1H), 7.80 (d, J = 8.4 Hz, 1H), | |
| | 7.67 (d, J = 8.4 Hz, 1H), 7.58 (t, J = 7.6 Hz, 1H), 7.50 (s, 1H), | |
| | 7.45 (t, J = 7.6 Hz, 1H), 7.11 (t, J = 56.4 Hz, 1H), 4.41 (br, 2H), | |
| | 3.95 (br, 2H), 3.70 (br, 2H), 3.45 (br, 2H), 3.36-3.30 (m, 2H), 3.12 | |
| | (br, 2H), 2.16-2.00 (m, 4H) | |
| 49 | ¹H NMR (400 MHz, DMSO-d6) δ 12.66 (s, 1H), 11.25 (br, 1H), | 544.65 |
| | 8.63 (d, J = 2.0 Hz, 1H), 7.94 (d, J = 8.4 Hz, 1H), 7.86 (dd, J = 8.4 | |
| | Hz, 1.6 Hz, 1H), 7.82-7.77 (m, 3H), 7.54 (t, J = 7.6 Hz, 2H), 7.50- | |
| | 7.45 (m, 3H), 4.73 (br, 2H), 3.97 (d, J = 12.0 Hz, 2H), 3.86 (t, J = | |
| | 12.0 Hz, 2H), 3.70-3.57 (m, 4H), 3.25-3.13 (m, 6H), 1.65 (t, J = | |
| | 6.4 Hz, 4H). | |
| 50 | ¹H NMR (400 MHz, CDCl₃) δ 8.37 (s, 1H), 8.16 (s, 1H), 7.78 (d, | 450.54 |
| | J = 8.4 Hz, 1H), 7.69-7.64 (m, 3H), 7.28 (s, 1H), 6.57 (br, 1H), | |
| | 3.83-3.80 (m, 7H), 2.12-2.05 (m, 2H). | |
| 51 | ¹H NMR (400 MHz, CDCl₃) δ 8.39 (d, J = 1.2 Hz, 1H), 8.10 (s, | 427.51 |
| | 1H), 7.92 (d, J = 8.0 Hz, 1H), 7.86-7.79 (m, 3H), 7.52 (t, J = 7.6 | |
| | Hz, 1H), 7.44 (t, .1 = 7.6 Hz, 1H), 3.82 (t, J = 7.6 Hz, 4H), 2.13- | |
| | 2.05 (m, 2H). | |
| 52 | ¹H NMR (400 MHz, DMSO-d6) δ 13.27 (s, 1H), 9.02 (d, J = 2.0 | 441.54 |
| | Hz, 1H), 8.65 (d, J = 4.8 Hz, 1H), 8.62 (s, 1H), 8.30 (d, J = 8.4 | |
| | Hz, 2H), 8.22 (d, J = 8.0 Hz, 1H), 7.99-7.96 (m, 3H), 7.88-7.86 | |
| | (m, 1H), 7.56-7.53 (m, 1H), 3.21-3.17 (m, 4H), 1.66-1.63 (m, 4H). | |
| 55 | ¹H NMR (400 MHz, DMSO-d6) δ 13.00 (s, 1H), 8.60 (d, J = 1.6 | 623.76 |
| | Hz, 1H), 8.00 (d, J = 8.4 Hz, 1H), 7.86-7.81 (m, 2H), 7.72 (d, J = | |
| | 1.6 Hz, 1H), 7.11 (d, J = 8.0 Hz, 1H), 6.18 (s, 2H), 3.70 (t, J = 7.6 | |
| | Hz, 4H), 2.01-1.94 (m, 2H). | |
| 56 | ¹H NMR (400 MHz, CDCl₃) δ 11.46 (s, 1H), 8.33 (s, 1H), 7.90- | 583.69 |
| | 7.82 (m, 2H), 7.71 (s, 1H), 6.65 (s, 1H), 6.07 (s, 2H), 4.27 (t, J = | |
| | 6.0 Hz, 2H), 3.74-3.65 (m, 4H), 3.33-3.25 (m, 4H), 2.58-2.42 (m, | |
| | 6H), 2.12-2.03 (m, 2H), 1.88-1.80 (m, 2H), 1.79-1.73 (m, 4H). | |
| 57 | ¹H NMR (400 MHz, DMSO-d6) δ 12.72 (s, 1H), 10.44 (br, 1H), | 6H.74 |
| | 8.62 (s, 1H), 8.34 (s, 1H), 8.01 (d, J = 8.4 Hz, 1H), 7.94-7.85 (m, | |
| | 3H), 7.63-7.56 (m, 2H), 7.48-7.44 (m, 1H), 4.91 (br, 1H), 4.26 | |
| | (br, 2H), 4.16 (s, 1H), 3.90-3.88 (m, 2H), 3.72-3.66 (m, 2H), 3.32- | |
| | 3.17 (m, 6H), 3.08-2.95 (m, 4H), 1.90 (br, 4H), 1.79-1.70 (m, 1H), | |
| | 1.67-1.60 (m, 1H). | |
| 58 | ¹H NMR (400 MHz, DMSO-d6) δ 12.36 (s, 1H), 9.45, (br, 1H), | 560.60 |
| | 8.34 (s, 1H), 8.01 (d, J = 8.0 Hz, 1H), 7.90 (d, J = 8.4 Hz, 1H), | |
| | 7.79 (d, J = 2.4 Hz, 1H), 7.74 (d, J = 8.8 Hz, 1H), 7.62-7.58 (m, | |
| | 1H), 7.55 (s, 1H), 7.46 (t, J = 7.2 Hz, 1H), 7.21 (dd, J = 8.8 Hz, | |
| | 2.8 Hz, 1H), 4.87-4.80 (q, 2H), 4.28 (br, 2H), 3.91 (t, J = 13.2 Hz, | |
| | 2H), 3.58 (t, J = 12.0 Hz, 2H), 3.39 (d, J = 13.2 Hz, 2H), 3.21 (br, | |
| | 2H), 3.07-2.99 (m, 2H), 1.89 (br, 4H). | |
| 59 | ¹H NMR (400 MHz, DMSO-d6) δ 12.42 (s, 1H), 10.22 (br, 1H), | 607.76 |
| | 8.63 (d, J = 1.6 Hz, 1H), 8.00 (d, J = 8.8 Hz 1H), 7.87-7.84 (m, | |
| | 2H), 7.87-7.79 (m, 2H), 7.55-7.52 (m, 2H), 7.48-7.44 (m, 3H), | |
| | 4.37-4.34 (m, 2H), 3.89 (d, J = 14.8 Hz, 2H), 3.70 (t, J = 7.6 Hz, | |
| | 414), 3.65 (d, J = 11.6 Hz, 2H), 3.36 (d, J = 12.0 Hz, 2H), 3.20 (br, | |
| | 2H), 3.06-2.96 (m, 2H), 2.02-1.96 (m, 2H), 1.91 (br, 4H). | |
| 60 | ¹H NMR (400 MHz, DMSO-d6) δ 12.68 (s, 1H), 10.97 (br, 1H), | 579.70 |
| | 8.63 (d, J = 1.6 Hz, 1H), 7.99 (d, J = 8.8 Hz 1H), 7.86-7.85 (m, | |
| | 2H), 7.80-7.78 (m, 2H), 7.56-7.52 (m, 2H), 7.51-7.45 (m, 3H), | |
| | 4.72 (br, 2H), 3.98 (d, J = 12.0 Hz, 2H), 3.83 (t, J = 7.6 Hz, 2H), | |
| | 3.71 (t, J = 7.6 Hz, 4H), 3.64-3.63 (m, 4H), 3.24-3.16 (m, 2H), | |
| | 2.02-1.95 (m, 2H). | |
| 61 | ¹H NMR (400 MHz, CDCl₃) δ 11.86 (br, 1H), 8.35 (d, J = 1.2 Hz, | 547.61 |
| | 1H), 7.88-7.80 (m, 2H), 7.70 (s, 1H), 6.62 (s, 1H), 6.06 (s, 2H), | |
| | 4.31 (t, J = 4.8 Hz, 2H), 3.83 (t, J = 7.6 Hz, 4H), 3.78 (t, J = 4.0 | |
| | Hz, 4H), 2.95 (t, J = 4.8 Hz, 2H), 2.67 (br, 4H), 2.13-2.05 (m, | |
| | 2H). | |
| 62 | ¹H NMR (400 MHz, CDCl₃) δ 12.06 (br, 1H), 8.81 (s, 1H), 8.42 | 559.69 |
| | (d, J = 1.2 Hz, 1H), 7.94-7.87 (m, 2H), 7.55 (s, 1H), 7.44 (s, 2H), | |
| | 4.47 (t, J = 5.2 Hz, 2H), 3.89-3.83 (m, 8H), 3.06 (t, J = 5.2 Hz, | |
| | 2H), 2.74 (br, 4H), 2.17-2.09 (m, 2H). | |
| 63 | ¹H NMR (400 MHz, CDCl₃) δ 11.64 (br, 1H), 8.79 (s, 1H), 8.38 | 585.77 |
| | (s, 1H), 7.91 (s, 2H), 7.54 (s, 1H), 7.40 (s, 2H), 4.40 (br, 2H), 3.84 | |
| | (t, J = 7.2 Hz, 2H), 3.72 (br, 2H), 2.60-2.55 (m, 4H), 2.22-1.92 | |
| | (m, 6H), 1.72-1.56 (m, 6H). | |
| 64 | ¹H NMR (400 MHz, DMSO-d₆) δ 12.06 (s, 1H), 9.89 (m, 1H), | 575.67 |
| | 8.61 (d, J = 1.6 Hz, 1H), 7.98 (d, J = 8.4 Hz, 1H), 7.84 (dd, J = 8.4 | |
| | Hz, 2.0 Hz, 1H), 7.35 (s, 1H), 7.06 (s, 1H), 6.14 (s, 2H), 4.22 (br, | |
| | 2H), 3.88 (d, J = 12.8 Hz, 2H), 3.70 (t, J = 7.6 Hz, 2H), 3.62 (t, | |
| | J = 12.8 Hz, 2H), 3.39-3.32 (m, 6H), 3.20 (br, 2H), 3.06-3.01 (m, | |
| | 2H), 2.02-1.96 (m, 2H), 1.86 (br, 2H). | |
| 65 | ¹H NMR (400 MHz, CDCl₃) δ 8.72 (s, 1H), 8.34 (s, 1H), 7.93 (d, | 520.62 |
| | J = 8.4 Hz, 1H), 7.85-7.83 (m, 2H), 7.78 (d, J = 8.0 Hz, 1H), 7.62- | |
| | 7.58 (m, 1H), 7.47 (t, J = 7.6 Hz, 1H), 7.29 (s, 1H), 4.66 (t, J = 4.0 | |
| | Hz, 2H), 4.60 (s, 4H), 4.00 (t, J = 4.4 Hz, 4H), 3.96 (s, 4H), 3.46 | |
| | (br, 2H),3.24 (br, 4H). | |
| 66 | ¹H NMR (400 MHz, DMSO-d6) δ 12.67 (s, 1H), 10.62 (br, 1H), | 520.62 |
| | 8.74 (d, J = 1.6 Hz, 1H), 8.34 (s, 1H), 8.06 (dd, J = 8.8, 2.0 Hz, | |
| | 1H), 8.01 (d, J = 8.0 Hz, 1H), 7.91 (d, J = 8.0 Hz, 1H), 7.86 (d, J = | |
| | 8.4 Hz, 1H), 7.63-7.58 (m, 1H), 7.56 (s, 1H), 7.49-7.43 (m, 1H), | |
| | 4.27 (t, J = 4.8 Hz, 2H), 3.90 (s, 3H), 3.86 (br, 2H), 3.72 (t, J = | |
| | 12.0 Hz, 2H), 3.41-3.37 (m, 2H), 3.17 (br, 2H), 3.03-2.95 (m, | |
| | 2H), 1.91 (br, 4H). | |
| 67 | ¹H NMR (400 MHz, CDCl₃) δ 12.14 (br, 1H), 8.39-8.34 (m, 2H), | 570.69 |
| | 7.87 (dd, J = 8.4, 1.6 Hz, 1H), 7.80 (d, .1 = 8.8 Hz, 1H), 7.26-7.24 | |
| | (m, 1H), 7,20 (s, 1H), 6.46 (d, J = 2.8 Hz, 1H), 4.40 (br, 2H), 3.87 | |
| | (s, 3H), 3.78 (br, 4H), 3.32-3.28 (m, 4H), 3.00 (br, 2H), 2.68 (br, | |
| | 4H), 1.80-1.76 (m, 4H). | |
| 69 | ¹H NMR (400 MHz, DMSO-d6) δ 13.82 (br, 1H), 10.25 (br, 1H), | 506.59 |
| | 8.75 (s, 1H), 8.33 (br, 1H), 8.10-7.98 (m, 2H), 7.96-7.79 (m, 2H), | |
| | 7.63 (br, 2H), 7.55-7.44 (m, 1H), 4.60 (br, 2H), 3.90 (br, 5H), | |
| | 3.80-3.49 (m, 6H), 3.25-3.11 (m, 2H). | |
| 77 | ¹H NMR (400 MHz, DMSO-d6) δ 8.61 (d, J = 1.60 Hz, 1H), 8.55 | 583.69 |
| | (s, 1H), 8.05 (d, J = 8.4 Hz, 1H), 7.91 (d, J = 8.4 Hz, 2H), 7.86 | |
| | (d, J = 2.0 Hz, 1H), 7.65-7.60 (m, 2H), 7.49-7.45 (m, 1H), 4.8 (br, | |
| | 1H), 4.44 (t, J = 5.2 Hz, 2H), 4.16 (br, 1H), 3.62-3.55 (m, 4H), | |
| | 3.27-3.20 (m, 4H), 3.08-3.05 (m, 1H), 2.86 (t, J = 5.2 Hz, 2H), | |
| | 2.55 (br, 4H), 1.81-1.70 (m, 1H), 1.68-1.59 (m, 1H). | |
| 78 | ¹H NMR (400 MHz, CDCl₃) δ 11.58 (br, 1H), 8.89 (s, 1H), 8.38 | 601.69 |
| | (s, 1H), 8.01-7.83 (m, 3H), 7.77 (d, J = 8.4 Hz, 1H), 7.69 (t, J = | |
| | 6.8 Hz, 1H), 7.4 (t, J = 7.2 Hz, 1H), 7.32 (s, 1H), 4.43-4.37(m, | |
| | 3H), 3.63 (br, 4H), 3.53-3.38 (m, 3H), 3.33-3.22 (m, 1H), 2.55- | |
| | 2.51 (m, 2H), 2.43 (br, 4H), 2.22-2.08 (m, 2H), 2.04-1.74 (m, 5H). | |
| 79 | ¹H NMR (400 MHz, CDCl₃) δ 11.88 (br, 1H), 8.90 (s, 1H), 8.37 | 601.70 |
| | (d, J = 1.6 Hz, 1H), 7.96 (d, J = 8.4 Hz, 1H), 7.88 (dd, J = 8.4, | |
| | 1.6 Hz, 1H), 7.82 (d, J = 8.4 Hz, 1H), 7.78 (d, J = 8.4 Hz, 1H), | |
| | 7.60 (t, J = 7.2 Hz, 1H), 7.48 (t, J = 7.2 Hz, 1H), 7.32 (s, 1H), | |
| | 4.46 (s, 2H), 3.32-3.28 (m, 4H), 3.06 (s, 2H), 2.82 (s, 4H), 2.35- | |
| | 2.14 (m, 4H), 1.86-1.72 (m, 4H). | |
| 80 | ¹H NMR (400 MHz, CDCl₃) δ 11.63 (s, 1H), 8.92 (s, 1H), 8.38 (d, | 629.75 |
| | J = 1.6 Hz, 1H), 7.96 (d, J = 8.0 Hz, 1H), 7.90 (dd, J = 8.4, 1.6 | |
| | Hz, 1H), 7.86 (d, J = 8.4 Hz, 1H), 7.78 (d, J = 8.4 Hz, 1H), 7.62- | |
| | 7.58 (m, 1H), 7.50-7.44 (m, 1H), 7.33 (s, 1H), 4.43 (t, J = 6.0 Hz, | |
| | 2H), 3.32-3.28 (m, 4H), 2.62 (br, 6H), 2.20-2.13 (m, 2H), 2.11- | |
| | 1.83 (m, 6H), 1.82-1.73 (m, 4H). | |
| 81 | ¹H NMR (400 MHz, DMSO-d₆) δ 12.05 (s, 1H), 8,43 (s, 1H), 8.17 | 463.58 |
| | (d, J = 8.4 Hz, 2H), 7.94-7.90 (m, 3H), 7.79 (d, J = 7.2 Hz, 2H), | |
| | 7.71 (dd, J = 8.4, 1.6 Hz, 1H), 7.53 (t, J = 7.6 Hz, 2H), 7.45 (t, J = | |
| | 7.6 Hz, 1H), 7.34 (s, 1H), 3.19-3.16 (t, J = 6.57 Hz, 4H), 1.70- | |
| | 1.58 (m, 4H). | |
| 82 | ¹H NMR (400 MHz, CDCl₃) δ 12.00 (s, 1H), 8.90 (s, 1H), 8.39 (d, | 583.69 |
| | J = 1.6 Hz, 1H), 7.95 (d, J = 8.4 Hz, 1H), 7.89 (dd, J = 8.4, 1.6 | |
| | Hz, 1H), 7.81-7.75 (m, 2H), 7.59 (t, J = 7.2 Hz, 1H), 7.46 (t, J = | |
| | 7.2 Hz, 1H), 7.31 (s, 1H), 4.47 (t, J = 5.05 Hz, 2H), 4.41 (s, 1H), | |
| | 3.89-3.75 (m, 4H), 3.51-3.40 (m, 3H), 3.32 (d, J = 10.8 Hz, 1H), | |
| | 3.04 (t, J = 5.2 Hz, 2H), 2.71 (br, 4H), 2.03-1.92 (m, 1H), 1.90- | |
| | 1.82 (m, 1H), 1.56-1.51 (m, 1H). | |
| 83 | ¹H NMR (400 MHz, CDCl₃) δ 11.59 (br, 1H), 8.89 (s, 1H), 8.39 | 6H.74 |
| | (d, J = 1.2 Hz, 1H), 7.95 (d, J = 8.0 Hz, 1H), 7.93-7.86 (m, 2H), | |
| | 7.78 (d, J = 8.4 Hz, 1H), 7.62-7.58 (m, 1H), 7.48-7.44 (m, 1H), | |
| | 7.33 (s, 1H), 4.43 (t, J = 6.0 Hz, 2H), 4.40-4.36 (m, 1H), 3.69- | |
| | 3.58 (m, 4H), 3.50-3.45(m, 2H), 3.44-3.39 (m, 1H), 3.28 (d, J = | |
| | 11.2 Hz, 1H), 2.57-2.53 (m, 2H), 2.45 (s, 4H), 2.19-2.12 (m, 2H), | |
| | 2.03-1.80 (m, 5H). | |
| 84 | ¹H NMR (400 MHz, DMSO-d6) δ 8.58 (br, 1H), 8.23-8.02 (m, | 573.71 |
| | 2H), 7.98-7.80 (m, 2H), 7.68-7.47 (m, 2H), 4.80 (br, 2H), 3.57 | |
| | (br, 4H), 3.19 (br, 4H), 2.90 (br, 2H), 2.61 (br, 4H), 1.66 (br, 4H). | |
| 24 | ¹H NMR (400 MHz, CDCl₃) δ 12.05 (s, 1H), 8.90 (s, 1H), 8.20 (s, | 459.54 |
| | 1H), 7.96 (d, J = 8.0 Hz, 1H), 7.78 (dd, J = 3.0, 8.2 Hz, 2H), 7.69 | |
| | (d, J = 8.4 Hz, 1H), 7.61 (t, J = 7.4 Hz, 1H), 7.47 (t, J = 7.6 Hz, | |
| | 1H), 7.32 (s, 1H), 4.47 (br, 2H), 3.87-3.77 (m, 4H), 3.08-3.00 (m, | |
| | 2H), 2.76-2.66 (m, 4H). | |
| 25 | ¹H NMR (400 MHz, DMSO-d6) δ 8.66 (s, 1H), 8.35 (s, 1H), 8.01 | 489.59 |
| | (d, J = 8.4 Hz, 1H), 7.94-7.87 (m, 3H), 7.61 (t, J = 7.6 Hz, 1H), | |
| | 7.56 (s, 1H), 7.46 (t, J = 7.4 Hz, 1H), 4.28-4.23 (m, 2H), 3.95-3.50 | |
| | (m, 4H), 3.20-2.70 (m, 4H), 2.40-2.10 (m, 2H), 1.91-1.81 (m, 4H). | |
| 54 | ¹H NMR (400 MHz, DMSO-d6) δ 12.58 (s, 1H), 11.26, (br, 11), | 532.55 |
| | 8.27 (s, 1H), 8.00 (d, J = 8.4 Hz, 1H), 7.93 (d, J = 8.4 Hz, 1H), | |
| | 7.79 (d, J = 2.8 Hz, 1H), 7.73 (d, J = 8.8 Hz, 1H), 7.64-7.60 (m, | |
| | 2H), 7.48 (t, J = 7.6 Hz, 1H), 7.21 (dd, J = 8.8 Hz, 2.8 Hz, 1H), | |
| | 4.87-4.79 (q, 2H), 4.66 (br, 2H), 3.93-3.80 (m, 4H), 3.64-3.61 (m, | |
| | 4H), 3.20-3.11 (m, 2H). | |
| 40 | ¹H NMR (400 MHz, DMSO-d6) δ 12.40 (s, 1H), 11.00 (br, 1H), | 561.64 |
| | 8.60 (d, J = 1.6 Hz, 1H), 7.91 (d, J = 8.4 Hz, 1H), 7.84 (dd, J = 8.4 | |
| | Hz, 1.6 Hz, 1H), 7.26 (s, 2H), 7.08 (s, 1H), 6.14 (s, 2H), 4.53 (br, | |
| | 2H), 4.00-3.90 (m, 2H), 3.83-3.76 (m, 2H), 3.66-3.54 (m, 4H), | |
| | 3.25-3.10 (m, 6H), 1.70-1.58 (m, 4H). | |
| 53 | ¹H NMR (400 MHz, DMSO-d6) δ 13.27 (s, 1H), 9.02 (d, J = 2.0 | 465.56 |
| | Hz, 1H), 8.65 (d, J = 4.8 Hz, 1H), 8.62 (s, 1H), 8.30 (d, J = 8.4 | |
| | Hz, 2H), 8.22 (d, J = 8.0 Hz, 1H), 7.99-7.96 (m, 3H), 7.88-7.86 | |
| | (m, 1H), 7.56-7.53 (m, 1H), 3.21-3.17 (m, 4H), 1.66-1.63 (m, 4H). | |

The following compounds in Table 5 were prepared as described above for N-(6-methanesulfonyl-1,3-benzothiazol-2-yl)naphthalene-2-carboxamide with the appropriate amine and carboxylic acid.

TABLE 5
Benzothiophenyl Compounds Prepared

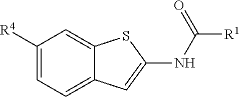

| | Amine | Acid | | | |
|---|---|---|---|---|---|
| Ex. | (Int. | (Int. | | | |
| No. | No.) | No.) | Name | R⁴ | R¹ |
| 68 | 25 | 40 | 3-[4-(morpholin-4- yl)butoxy]-N-[6- (pyrrolidine-1-sulfonyl)- 1-benzothiophen-2-yl]- [1,1′-biphenyl]-4- carboxamide | 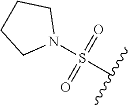 [SMILES: *S(=O)(=O)N1CCCC1] | 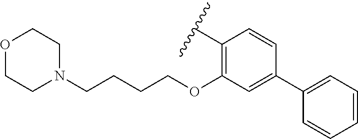 [SMILES: *c1ccc(-c2ccccc2)cc1OCCCCN1CCOCC1] |
| 70 | 25 | 36 | 3-[4-(morpholin-4- yl)butoxy]-N-[6- (pyrrolidine-1-sulfonyl)- 1-benzothiophen-2- yl]naphthalene-2- carboxamide | 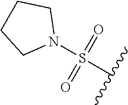 [SMILES: *S(=O)(=O)N1CCCC1] | 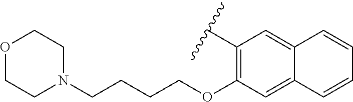 [SMILES: *c1cc2ccccc2cc1OCCCCN1CCOCC1] |
| 71 | 25 | 39 | 3-[2-(morpholin-4- yl)ethoxy]-N-[6- (pyrrolidine-1-sulfonyl)- 1-benzothiophen-2-yl]- [1,1′-biphenyl]-4- carboxamide | 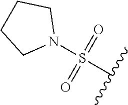 [SMILES: *S(=O)(=O)N1CCCC1] | 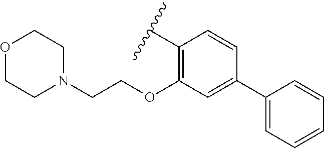 [SMILES: *c1ccc(-c2ccccc2)cc1OCCN1CCOCC1] |
| 72 | 25 | 42 | 6-[4-(morpholin-4- yl)butoxy]-N-[6- (pyrrolidine-1-sulfonyl)- 1-benzothiophen-2-yl]- 1-benzothiophene-5- carboxamide | 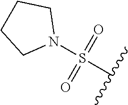 [SMILES: *S(=O)(=O)N1CCCC1] | 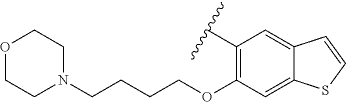 [SMILES: *c1cc2ccsc2cc1OCCCCN1CCOCC1] |
| 73 | 25 | 44 | 6-[4-(morpholin-4- yl)butoxy]-N-[6- (pyrrolidine-1-sulfonyl)- 1-benzothiophen-2-yl]- 2H-1,3-benzodioxole-5- carboxamide | 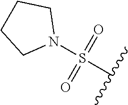 [SMILES: *S(=O)(=O)N1CCCC1] | 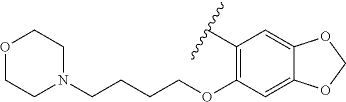 [SMILES: *c1cc2c(cc1OCCCCN1CCOCC1)OCO2] |
| 74 | 25 | 35 | 3-[2-(morpholin-4- yl)ethoxy]-N-[6- (pyrrolidine-1-sulfonyl)- 1-benzothiophen-2-yl] naphthalene-2- carboxamide | 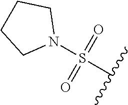 [SMILES: *S(=O)(=O)N1CCCC1] | 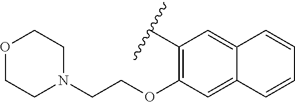 [SMILES: *c1cc2ccccc2cc1OCCN1CCOCC1] |
| 75 | 25 | 43 | 3-[2-(morpholin-4- yl)ethoxy]-N-[6- (pyrrolidine-1-sulfonyl)- 1-benzothiophen-2- yl]naphthalene-2- carboxamide | 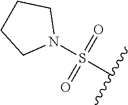 [SMILES: *S(=O)(=O)N1CCCC1] | 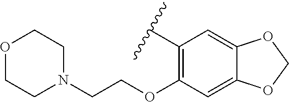 [SMILES: *c1cc2c(cc1OCCN1CCOCC1)OCO2] |
| 76 | 25 | 41 | 6-[2-(morpholin-4- yl)ethoxy]-N-[6- (pyrrolidine-1-sulfonyl)- 1-benzothiophen-2-yl]- 1-benzothiophene-5- carboxamide | 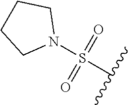 [SMILES: *S(=O)(=O)N1CCCC1] | 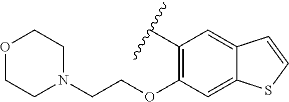 [SMILES: *c1cc2ccsc2cc1OCCN1CCOCC1] |

### Example 85

### Synthesis of N-(6-(((2R,5S)-2,5-dimethylpyrrolidin-1-yl)sulfonyl)benzo[d]thiazol-2-yl)-3-(4-morpholinobutoxy)-2-naphthamide

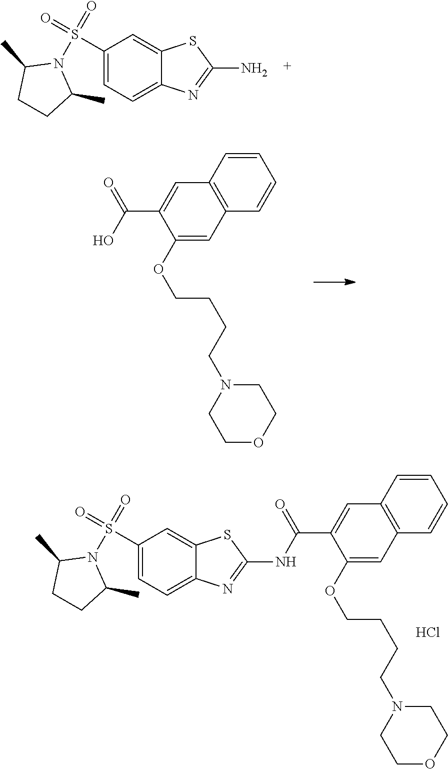 [SMILES: C[C@@H]1CC[C@H](C)N1S(=O)(=O)c1ccc2nc(N)sc2c1 ; O=C(O)c1cc2ccccc2cc1OCCCCN1CCOCC1 ; C[C@@H]1CC[C@H](C)N1S(=O)(=O)c1ccc2nc(NC(=O)c3cc4ccccc4cc3OCCCCN3CCOCC3)sc2c1 ; Cl]

To a solution of the amine (150 mg, 0.482 mmol, 1.0 eq.) and acid, which is 3-[4-(morpholin-4-yl)butoxy]naphthalene-2-carboxylic acid (intermediate 39) (175 mg, 0.530 mmol, 1.1 eq), (3-[4-(morpholin-4-yl)butoxy]naphthalene-2-carboxylic acid) was added MeCN/DMF (0.068 M, ratio=5/2). 1-Methylimidazole (119 mg, 1.44 mmol, 0.115 mL, 3.0 eq.) was added at room temperature followed by N,N,N′,N′-tetramethylchloroformamidinium hexafluorophosphate (TCFH) (162 mg, 0.578 mmol, 1.20 eq.). The mixture was heated to 40° C. for 18 h. The reaction was then cooled to room temperature and diluted with water (5 mL). The reaction was stirred and then the solid was collected by filtration. The solid was dried under vacuum oven, and then triturated with ethanol (5 mL). After collection of a solid, the product was taken up in ethanol (3 mL), and HCl (4N in dioxane) was added, and the mixture was stirred rigorously for 30 min, filtered, and dried under vacuum to afford the product (185 mg, 58.3%) as a beige solid. ¹H NMR (400 MHz, DMSO-d₆) δ 12.71 (s, 1H), 9.78 (s, 1H), 8.67 (d, J=1.8 Hz, 1H), 8.34 (s, 1H), 8.01 (d, J=8.1 Hz, 1H), 7.95-7.81 (m, 2H), 7.67-7.53 (m, 2H), 7.46 (ddd, J=8.2, 6.8, 1.2 Hz, 1H), 4.28 (d, J=5.5 Hz, 2H), 3.89 (d, J=12.7 Hz, 2H), 3.74-3.54 (m, 4H), 3.49-3.37 (m, 2H), 3.20 (s, 2H), 3.01 (q, J=11.3 Hz, 2H), 1.89 (s, 5H), 1.64-1.41 (m, 4H), 1.30 (d, J=6.3 Hz, 6H).

LCMS (acid method) M+H Calculated for C₃₂H₃₈N₄O₅S₂=622.23, Found: 623.5.

### Example 86

### Synthesis of [N-(6-(azetidin-1-ylsulfonyl)benzo[b]thiophen-2-yl)-3-(4-morpholinobutoxy)-2-naphthamide]

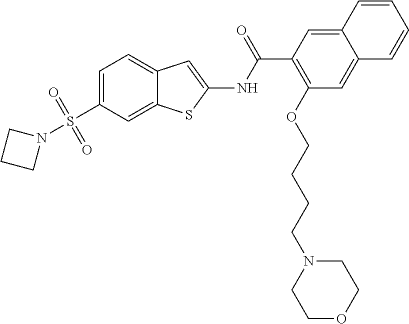 [SMILES: O=C(Nc1cc2ccc(S(=O)(=O)N3CCC3)cc2s1)c1cc2ccccc2cc1OCCCCN1CCOCC1]

A mixture of 3-(4-morpholinobutoxy)-2-naphthoic acid, intermediate 39 herein, (102 mg, 0.31 mmol) in SOCl₂ (1 mL) was stirred at 70° C.-80° C. for 1.5 h. After SOCl₂ was removed, the residue was dissolved in DCM (1 mL) and added dropwise into a mixture of TEA (80 mg, 0.78 mmol) and 6-(azetidin-1-ylsulfonyl) benzo[b]thiophen-2-amine, intermediate 21 herein, (70 mg, 0.26 mmol) in DCM (1 mL). The resulting mixture was stirred at room temperature for 6 h, and then quenched with saturated aq. NaHCO₃ solution and extracted with DCM. The organic phase was dried over Na₂SO₄ and concentrated under reduced pressure to give a residue which was purified through silica gel flash column chromatography (Eluent: DCM/MeOH=50/1) to afford the title compound as a yellow solid (78 mg, 52%).

LC_MS (ES⁺) calcd for C₃₀H₃₃N₃O₅S₂: 579.7; found: 580.5 [M+H].

¹H NMR (400 MHz, CDCl₃) δ 11.11 (s, 1H), 8.90 (s, 1H), 8.31 (s, 1H), 7.94 (d, J=8.0 Hz, 1H), 7.80-7.73 (m, 3H), 7.61-7.55 (m, 1H), 7.48-7.42 (m, 1H), 7.30 (s, 1H), 7.00 (s, 1H), 4.41 (t, J=6.4 Hz, 2H), 3.82 (t, J=7.6 Hz, 4H), 3.77-3.65 (m, 4H), 2.61-2.40 (m, 6H), 2.22-2.12 (m, 2H), 2.11-2.03 (m, 2H), 1.91-1.83 (m, 2H).

NMR and LCMS mass spectrometry data for the compounds of Table 5 are provided below in Table 6.

TABLE 6
Characterization Data for Benzothiophenyl Compounds

| Ex. | | LCMS |
|---|---|---|
| No. | ¹H NMR | (MH⁺) |
| 68 | ¹H NMR (400 MHz, CDCl₃) δ 10.93 (s, 1H), 8.36 (d, J = 8.4 Hz, 1H), | 620.8 |
| | 8.29 (s, 1H), 7.76 (dd, J = 8.4, 1.6 Hz, 1H), 7.70 (d, J = 8.4 Hz, 1H), 7.64- | |
| | 7.61 (m, 2H), 7.49 (t, J = 7.2 Hz, 2H), 7.46-7.41 (m, 1H), 7.39 (dd, J = | |
| | 8.4, 1.2 Hz, 1H), 7.23 (d, J = 0.8 Hz, 1H), 6.97 (s, 1H), 4.39 (t, J = 6.4 | |
| | Hz, 2H), 3.71 (br, 4H), 3.30-3.27 (m, 4H), 2.53-2.45 (m, 6H), 2.16-2.09 | |
| | (m, 2H), 1.87-1.81 (m, 2H), 1.74-1.73 (m, 414). | |
| 70 | ¹H NMR (400 MHz, DMSO-d6) δ 11.99 (s, 1H), 10.34 (br, 1H), 8.45 (s, | 594.8 |
| | 1H), 8.24 (s, 1H), 7.99 (d, J = 8.0 Hz, 1H), 7.91 (dd, J = 8.4, 5.6 Hz, | |
| | 2H), 7.71 (dd, J = 8.4, 1.6 Hz, 1H), 7.59 (s, 1H), 7.55 (s, 1H), 7.45 (t, J = | |
| | 7.2 Hz, 1H), 7.23 (s, 1H), 4.25 (s, 2H), 3.80-3.77 (m, 2H), 3.64 (t, J = 7.2 | |
| | Hz, 2H), 3.31-3.26 (m, 2H), 3.23-3.08 (m, 6H), 2.98-2.87 (m, 2H), 1.87 | |
| | (br, 4H), 1.66-1.62 (m, 4H). | |
| 71 | ¹H NMR (400 MHz, DMSO-d6) δ 12.00 (s, 1H), 10.98 (s, 1H), 8.44 (s, | 592.7 |
| | 1H), 7.91 (d, J = 8.4 Hz, 1H), 7.80 (d, J = 7.2 Hz, 2H), 7.74-7.69 (m, 2H), | |
| | 7.57-7.49 (m, 3H), 7.47-7.43 (m, 2H), 7.27 (s, 1H), 4.69 (br, 2H), 3.92- | |
| | 3.80 (m, 4H), 3.70-3.51 (m, 4H), 3.22-3.14 (m, 6H), 1.65-1.62 (m, 4H). | |
| 72 | ¹H NMR (400 MHz, DMSO-d6) 11.85 (s, 1H), 10.34 (br, 1H), 8.44 (s, | 600.8 |
| | 1H), 8.16 (s, 1H), 7.90 (d, J = 8.4 Hz, 1H), 7.86 (s, 1H), 7.71 (dd, J = | |
| | 8.4, 1.4 Hz, 1H), 7.68 (d, J = 5.2 Hz, 1H), 7.48 (d, J = 6.0 Hz, 1H), 7.23 | |
| | (s, 1H), 4.21 (br, 2H), 3.80-3.76 (m, 2H), 3.66-3.59 (m, 2H), 3.29-3.23 | |
| | (m, 2H), 3.21-3.06 (m, 6H), 2.97-2.84 (m, 2H), 1.85 (br, 4H), 1.66-1.61 | |
| | (m, 4H). | |
| 73 | ¹H NMR (400 MHz, DMSO-d6) δ 11.50 (s, 1H), 10.37 (br, 1H), 8.41 (d, | 588.7 |
| | J = 8,4 Hz, 1H), 7.70 (s, 1H), (dd, J = 8.4, 1.6 Hz, 1H), 7.22 (d, J = 5.6 | |
| | Hz, 2H), 7.02 (s, 1H), 6.11 (s, 2H), 4.14 (br, 2H), 3.79-3.76 (m, 2H), 3.62 | |
| | (d, J = 11.6 Hz, 2H), 3.25 (d, J = 12.8 Hz, 2H), 3.18-3.14 (m, 4H), 3.14- | |
| | 3.07 (m, 2H), 2.96-2.85 (m, 2H), 1.81 (br, 4H), 1.68-1.57 (m, 4H). | |
| 74 | ¹H NMR (400 MHz, DMSO-d6) δ 12.22 (s, 1H), 11.41 (br, 1H), 8.46 (s, | 567.7 |
| | 1H), 8.25 (s, 1H), 8.01 (d, J = 8.4 Hz, 1H), 7.94-7.90 (m, 2H), 7.71 (dd, | |
| | J = 8.4, 1.6 Hz, 1H), 7.65-7.58 (m, 2H), 7.51-7.45 (m, 1H), 7.27 (s, 1H), | |
| | 4.67 (s, 2H), 3.87 (br, 4H), 3.63-3.56 (m, 4H), 3.21-3.15 (m, 6H), 1.68- | |
| | 1.60 (m, 4H). | |
| 75 | ¹H NMR (400 MHz, DMSO-d6) δ 11.48 (br, 1H), 8.28 (s, 1H), 7.75 (d, | 560.7 |
| | J = 8.4 Hz, 1H), 7.62(d, J = 8.0 Hz, 1H), 7.29 (s, 1H), 7.01 (br, 1H), 6.97 | |
| | (s, 1H), 6.08 (s, 2H), 4.25 (t, J = 5.2 Hz, 2H), 3.53-3,44 (m, 4H), 3.18- | |
| | 3.13 (m, 4H), 2.76 (t, J = 4.8 Hz, 2H), 2.47-2.44 (m, 4H), 1.65-1.61 (m, | |
| | 4H). | |
| 76 | ¹H NMR (400 MHz, DMSO-d6) δ 12.08 (s, 1H), 10.91(br, 1H), 8.45 (s, | 572.71 |
| | 1H), 8.16 (s, 1H), 7.90-7.94 (m, 2H), 7.73-7.69 (m, 2H), 7.50 (d, J = 5.6 | |
| | Hz, 1H), 7.24 (s, 1H), 4.60 (s, 2H), 3.92-3.81 (m, 4H), 3.62-3.56 (m, 4H), | |
| | 3.19-3.17 (m, 6H), 1.65-1.62 (m, 4H). | |

### Example A: Induction of IRF3-Dependent Gene Expression in THP1-Lucia™ ISG Cells

The compounds were evaluated in the THP1-Lucia™ ISG (interferon stimulated genes) reporter assay to determine if the compounds activate the IRF3 signaling pathway. The THP1-Lucia™ cells (InvivoGen) express the secreted luciferase (Lucia) reporter gene under the control of an IRF-inducible promotor. The reporter cell line was developed from human monocytic leukemia THP-1 cells.

The promotor was comprised of five IFN-stimulated response elements (ISRE) fused to an ISG54 minimal promotor which is unresponsive to NF-kB or AP-1 pathways. The secretion of luciferase by the THP1-Lucia™ ISG reporter cell line in response to small molecule RIG-I agonist compounds indicated the activation of the IRF3 pathway, since IRF3-deficient THP1-Lucia™ ISG IRF3 −/− cells do not induce the secretion of luciferase in response to compounds. The IRF3-deficient THP1-Lucia™ ISG IRF3 −/− reporter cell line was generated by CRISPR technology from the parent THP1-Lucia™ ISG reporter cell line.

5 THP1-Lucia™ ISG cells and IRF3-deficient THP1-Lucia™ ISG IRF3 −/− cells were differentiated with PMA (100 ng/ml) and stimulated with compounds at the indicated concentrations (5 to 20 μM), positive control, or not treated (background). Luciferase secretion was quantified using the QUANTI-Luc luciferase assay system (InvivoGen) 18 h after stimulation. Data are shown as fold increase luciferase activity over background in Table 7 and represent the IRF3-dependent ISG54 promotor activity by the THP1-Lucia™ ISG cells in response to compounds. None of the listed 84 compounds induced luciferase expression in the IRF3 deficient THP1-Lucia™ IRF3 −/− cells. The fold increase of compounds (10 μM, *20 μM, **5 μM) induced IRF3 dependent luciferase activity is indicated as follows: “−” indicates less than 2.4 fold increase; “+” indicates a 2.4-4.9 fold increase; “++” indicates a 5-9.9 fold increase; “+++” indicates a 10-19 fold increase; “++++” indicates a 20-39 fold increase; “+++++” indicates greater than or equal to 40 fold increase.

TABLE 7
Compound induced fold increase of IRF3-depedent luciferase activity

| | | THP1 |
|---|---|---|
| | THP1 | ISG IRF3 |
| Ex. | ISG | −/− |
| 1 | +++ | − |
| 2 | +++++ | − |
| 3 | +++ | − |
| 4 | +++++ | − |
| 5 | + | − |
| 6 | ++++ | − |
| 7 | ++++ | − |
| 8 | +++ | − |
| 9 | ++ | − |
| 10 | ++ | − |
| 11 | +++ | − |
| 12 | +* | −* |
| 13 | ++++ | − |
| 14 | ++++ | − |
| 15 | ++++ | − |
| 16 | ++++ | − |
| 17 | +++ | − |
| 18 | + | − |
| 19 | ++ | − |
| 20 | +++ | − |
| 21 | ++ | − |
| 22 | +** | −** |
| 23 | ++** | −** |
| 24 | ++ | − |
| 25 | +* | −** |
| 26 | ++++ | − |
| 27 | ++++ | − |
| 28 | + | − |
| 29 | +++++ | − |
| 30 | +++ | − |
| 31 | +++ | − |
| 32 | ++** | −** |
| 33 | + | − |
| 34 | + | − |
| 35 | ++++ | − |
| 36 | +++ | − |
| 37 | +++ | − |
| 38 | +++ | − |
| 39 | ++++ | − |
| 40 | +* | −* |
| 41 | ++++ | − |
| 42 | ++ | − |
| 43 | ++ | − |
| 44 | − | − |
| 45 | ++ | − |
| 46 | +++* | −* |
| 47 | +++++ | − |
| 48 | +* | −* |
| 49 | ++++ | − |
| 50 | ++ | − |
| 51 | + | − |
| 52 | +++ | − |
| 53 | + | − |
| 54 | + | − |
| 55 | + | − |
| 56 | +++ | − |
| 57 | +++ | − |
| 58 | ++ | − |
| 59 | +++ | − |
| 60 | ++++ | − |
| 61 | ++ | − |
| 62 | ++++ | − |
| 63 | + | − |
| 64 | +++ | − |
| 65 | + | − |
| 66 | ++** | −** |
| 67 | ++++ | − |
| 68 | ++++ | − |
| 69 | ++** | −** |
| 70 | +++ | − |
| 71 | − | − |
| 72 | ++++ | − |
| 73 | ++++ | − |
| 74 | +++ | − |
| 75 | ++ | − |
| 76 | ++++ | − |
| 77 | + | − |
| 78 | +++++ | −** |
| 80 | ++++ | − |
| 81 | ++++ | − |
| 82 | − | − |
| 83 | ++++ | − |
| 84 | ++ | − |
| 86 | ++++ | − |
| 87 | ++++ | − |
| 88 | ++++ | − |
| 89 | +++ | − |
| 90 | ++++ | − |
| 91 | +++ | − |

* = 20 μM,
** = 5 μM compound concentrations.
All other compounds were evaluated at 10 μM

### Example B: Induction of RIG-I Dependent CXCL10 Secretion by Murine CT26 Colon Carcinoma Cells in Response to Compounds

The CT26 murine colon carcinoma cell line (ATCC) was used to evaluate the induction of CXCL10 secretion. CXCL10 is an important chemokine in tumor immune biology that recruits tumor-specific T cells to the tumor. To confirm that compound-mediated CXCL10 production was RIG-I specific, RIG-I deficient CT2-RIG-1−/− cells were generated by Kineta using CRISPR technology.

CT26 cells were seeded at a density of 1×10⁴ cells per well on a 96-well tissue culture plate in 100 μL of cell culture and cells were incubated at 37° C. and 5% CO₂ for 24 hr. Next, CT26 cells were treated with compounds at the indicated concentrations. CXCL10 was quantified by ELISA from supernatants taken 24 h after compound stimulation by use of the CXCL10 DuoSet ELISA kit (Cat # DY466, R&D, Minneapolis, Minn., USA) according to the manufacturer's instructions.

CXCL10 secretion by CT26 cells in response to compounds (in an amount of 5 to 20 μM) of the present disclosure is shown in Table 8. The compound-induced CXCL10 production was RIG-I dependent, since none of the compounds mediated CXCL10 secretion in RIG-I deficient CT26 RIG-1−/− cells. The compounds (10 μM, *20 μM, **5 μM) are indicated in the table as follows: “−” indicates less than 100 pg/mL; “+” indicates 100-199 pg/mL; “++” indicates 200-399 pg/mL; “+++” indicates 400-799 pg/mL; “++++” indicates 800 to 1599 pg/mL; “+++++” indicates greater than or equal to 1600 pg/ml.

TABLE 8
RIG-I dependent CXCL10 secretion by murine CT26
colon carcinoma cells in response to compounds

| | | CT26 |
|---|---|---|
| | | RIG-I |
| Ex. | CT26 | −/− |
| 1 | ++++ | − |
| 2 | ++++ | − |
| 3 | +++ | − |
| 4 | ++++ | − |
| 5 | +++ | − |
| 6 | ++ | − |
| 7 | ++ | − |
| 8 | ++++ | − |
| 9 | +++ | − |
| 10 | +++ | − |
| 11 | ++ | − |
| 12 | +++ | − |
| 13 | +++++ | − |
| 14 | ++++ | − |
| 15 | ++++ | − |
| 16 | ++++ | − |
| 17 | +++ | − |
| 18 | + | − |
| 19 | ++++ | − |
| 20 | +** | −** |
| 21 | +** | −** |
| 22 | +** | −** |
| 23 | ++++** | −** |
| 24 | − | − |
| 25 | +* | −* |
| 26 | +* | −* |
| 27 | +++ | − |
| 28 | + | − |
| 29 | ++++ | − |
| 30 | +++ | − |
| 31 | +++++ | − |
| 32 | ++++ | − |
| 33 | ++** | −** |
| 34 | +++ | − |
| 35 | + | − |
| 36 | +++ | − |
| 37 | +++ | − |
| 38 | +++ | − |
| 39 | ++ | − |
| 40 | ++** | −** |
| 41 | +++++ | − |
| 42 | ++++ | − |
| 43 | ++++ | − |
| 44 | ++** | −** |
| 45 | ++++ | − |
| 46 | ++ | − |
| 47 | +++ | − |
| 48 | ++ | − |
| 49 | +++ | − |
| 50 | +++++ | − |
| 51 | + | − |
| 52 | +* | −* |
| 53 | − | − |
| 54 | − | − |
| 55 | ++ | − |
| 56 | +++ | − |
| 57 | +++ | − |
| 58 | ++ | − |
| 59 | +++ | − |
| 60 | ++++ | − |
| 61 | ++ | − |
| 62 | ++++ | − |
| 63 | + | − |
| 64 | +++ | − |
| 65 | + | − |
| 66 | ++** | −** |
| 67 | ++++ | − |
| 68 | ++++ | − |
| 69 | ++** | −** |
| 70 | +++ | − |
| 71 | − | − |
| 72 | ++++ | − |
| 73 | ++++ | − |
| 74 | +++ | − |
| 75 | ++ | − |
| 76 | ++++ | − |
| 77 | + | − |
| 78 | +++++ | − |
| 79 | +** | −** |
| 80 | ++++ | − |
| 81 | ++++ | − |
| 82 | − | − |
| 83 | ++++ | − |
| 84 | ++ | − |

* = 20 μM,
** = 5 μM compound concentrations.
All other compounds were evaluated at 10 μM

### Example C: Compound-Induced Immunogenic Cell Death in Murine Colon Carcimoma Cells

To determine if the RIG-I agonist compounds induce immunogenic cell death in cancer cells, induction of apoptosis and the translocation or of calreticulin (CRT) to the cell surface in murine CT26 colon carcinoma cells were evaluation. The translocation of CRT occurs as part of a specific RIG-I dependent danger-signaling system, and the presence of CRT on the cell membrane promotes tumor antigen uptake by the dendritic cells and leads to the induction of am antigen-specific T cell response

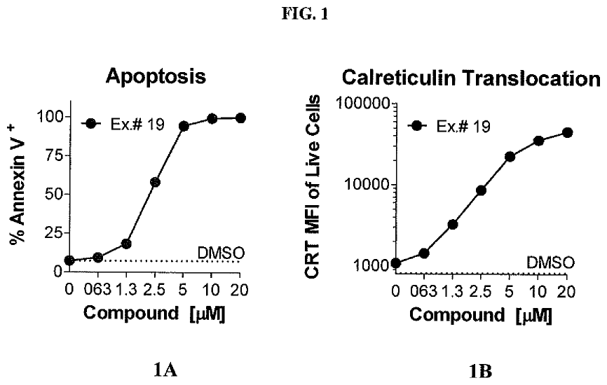

The induction of apoptosis and the CRT translocation was measured by flow cytometry. CT26 cells were seeded at a density of 4×10⁴ cells per well of a 6-well tissue culture plate in 2 mL of cell culture media and cells were incubated for 24 hr. Next, CT26 cells were treated with compounds at the indicated concentrations or treated with DMSO control (FIG. 1). Cells were harvested 18 h after treatment and then prepared for flow cytometry using an Annexin V staining kit (Biolegend) for quantification of apoptosis, an anti-CRT antibody (Abgent) for calreticulin translocation, and the Live/Dead-Violet staining kit (Thermofisher) for cell viability. Induction of apoptosis and translocation of calreticulin (CRT) to cell surface by live cells was determined by tri-color flow cytometry using FITC-labeled Annexin V, Live/Dead-Violet (LDV), and APC-anti-CRT. Apoptotic cells were defined as Annexin V⁺ and calreticulin translocation to cell surface was quantified by mean fluorescent intensity (MFI) of calreticulin⁺ live cells (CRT⁺ LDV⁻). A representative example of the induction of immunogenic cell death is shown in FIG. 1 for the compound of Example 19. The data represent typical dose titrations for induction apoptosis and calreticulin translocation by immunogenic cell death inducing compounds of this invention.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula (I): 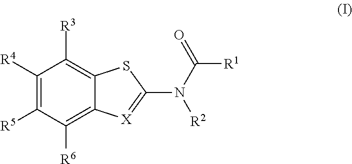
or a pharmaceutically acceptable salt thereof, wherein:
X is N or CR^{X};
R^{X} is H or C₁₋₆ alkyl;
R¹ is a group having Formula (i), (ii), (iii), (iv) or (v): 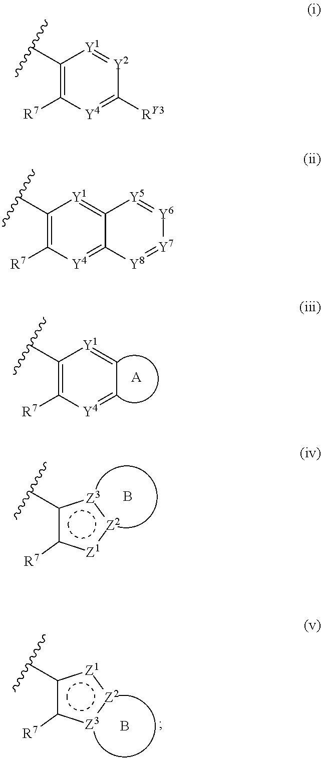
Y¹ is N or CR^{Y1};
Y² is N or CR^{Y2};
Y⁴ is N or CR^{Y4};
Y⁵ is N or CR^{Y5};
Y⁶ is N or CR^{Y6};
Y⁷ is N or CR^{Y7};
Y⁸ is N or CR^{Y8};
wherein not more than four of Y¹, Y², Y⁴, Y⁵, Y⁶, Y⁷, and Y⁸ in formula (ii) are N;
Z¹ is N, CR^{Z1}, O, S, or NR^{Z1};
Z² is N or C;
Z³ is N or C;
Ring A is a fused 5-membered heteroaryl group or a fused 4-7 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C_{2_6} alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
Ring B is a fused phenyl, C₃₋₇ cycloalkyl, 5-6 membered heteroaryl, or 4-7 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1},
R^{Y1}, R^{Y2}, R^{Y4}, R^{Y5}, R^{Y6}, R^{Y7}, R^{Y8}, and R^{Z1} are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{Y1}, R^{Y2}, R^{Y4}, R^{Y5}, R^{Y6}, R^{Y7}, R^{Y8}, and R^{Z1} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1},
R^{Y3} is phenyl, C₃₋₇ cycloalkyl, 5-6 membered heteroaryl, or 4-7 membered heterocycloalkyl, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}C(S)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2},
R² is H or C₁₋₄ alkyl;
R³ is H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R³ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR, NR^{c3}C(O)NR^{c3}R^{d3}, NR^{c3}C(S)NR^{c3}R^{d3},NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3},
R⁴ is H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R³ are each optionally substituted with 1, 2, 3, 4, or 5 substituents independently selected from Cy⁴, Cy⁴-C₁₋₄ alkyl, halo, C₁₋₆ alkyl, C₂₋₆ alkenyl, C₂₋₆ alkynyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
wherein at least one of R³ and R⁴ is other than H;
each R⁵ and R⁶ is independently selected from H, halo, C₁₋₆ alkyl, C₁₋₆ haloalkyl, C₆₋₁₀ aryl, CN, NO₂, OR^{a5}, SR^{a5}, C(O)R^{b5}, C(O)NR^{c5}R^{d5}, C(O)OR^{a1}, OC(O)R^{b5}, OC(O)NR^{c5}SR^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5},
R⁷ is a group having the formula: —(C₁₋₂ alkyl)ₐ-(L¹)_{b}-(C₂₋₆ alkyl)_{c}-(L²)_{d}-Q;
L is —O—, —S—, —NR⁸—, —CO—, —C(O)O—, —CONR⁸—, —SO—, —SO₂—, —SONR⁸—, —SO₂NR—, or —NR⁸CONR⁹—;
L² is —O—, —S—, —NR¹⁰—, —CO—, —C(O)O—, —CONR¹⁰—, —SO—, —SO₂—, —SONR¹⁰—, —SO₂NR⁸—, or —NR¹⁰CONR¹¹—;
R⁸, R⁹, R¹⁰, and R¹¹ are each independently selected from H and C₁₋₄ alkyl;
a is 0 or 1;
b is 0 or 1;
c is 0 or 1;
d is 0 or 1;
wherein the sum of b and d is 1 or 2;
wherein the sum of a and c is 1 or 2;
Q is 5-6 membered heteroaryl or 5-7 membered heterocycloalkyl, each optionally substituted by 1, 2, 3 or 4 substituents selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a}, C(O)R^{b}, C(O)NR^{c}R^{d} C(O)OR^{a1}, OC(O)R^{b}, OC(O)NR^{c}R^{d}, C(═NR^{e})NR^{c}R^{d}, NR^{c}C(═NR^{e1})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy⁴ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, S(O)₂NR^{c6}R^{d6}, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is optionally substituted with 1, 2, 3, 4, or 5 substituents independently selected from Cy⁶, Cy⁶-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6},
or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c2} and R^{d2} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c3} and R^{d3} together with the N atom to which they are attached form a 3-7 membered 3-7 heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c4} and R^{d4} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a5}, R^{b5}, R^{c5}, and R^{d5} is independently selected from H and C₁₋₆ alkyl;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e}, R^{e1}, R^{e2}, R^{e3}, R^{e4}, R^{e5}, and R^{e6} is independently selected from H, C₁₋₄ alkyl, and CN,
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group;
wherein one or more ring-forming S atoms of any aforementioned heterocycloalkyl group is optionally substituted by one or two oxo (═O) groups.

2. The compound of claim 1, or a pharmaceutically acceptable salt thereof, wherein:
X is N or CR^{X};
R^{X} is H or C₁₋₆ alkyl;
R¹ is a group having Formula (i), (ii), or (iii): 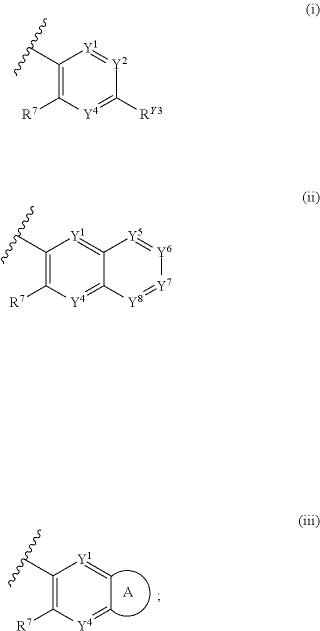
Y¹ is CR^{Y1};
Y² is CR^{Y2};
Y⁴ is CR^{Y4};
Y⁵ is CR^{Y5};
Y⁶ is CR^{Y6};
Y⁷ is CR^{Y7};
Y⁸ is CR^{Y8};
Ring A is a fused 5-membered heteroaryl group or a fused 4-7 membered heterocycloalkyl group, each optionally substituted with 1, 2, 3, 4, or 5 substituents independently selected from Cy¹, halo, C₁₋₆ alkyl, C₁₋₆ haloalkyl, CN, NO₂, OR^{a1}, SR^{a1}, C(O)R^{b1}, C(O)NR^{c1}R^{d1}, and C(O)OR^{a1}, wherein the C₁₋₆ alkyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, S(O)R^{b1}, S(O)₂R^{b1}, and S(O)₂NR^{c1}R^{d1},
R^{Y1}, R^{Y2}, R^{Y4}, R^{Y5}, R^{Y6}, R^{Y7}, R^{Y8}, and R^{Z1} are each independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl of R^{Y1}, R^{Y2}, R^{Y4}, R^{Y5}, R^{Y6}, R^{Y7}, R^{Y8}, and R^{Z1} are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, and S(O)₂R^{b1};
R^{Y3} is phenyl, C₃₋₇ cycloalkyl, 5-6 membered heteroaryl, or 4-7 membered heterocycloalkyl, each optionally substituted with 1, 2, 3, 4, or 5 substituents independently selected from Cy², Cy²-C₁₋₄ alkyl, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, NR^{c2}R^{d2}, NR^{c2}C(O)R^{b2}, S(O)R^{b2}, S(O)NR^{c2}R^{d2}, S(O)₂R^{b2}, and S(O)₂NR^{c2}R^{d2};
R² is H or C₁₋₄ alkyl;
R³ is H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, and C₁₋₆ haloalkyl, of R³ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3},NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
R⁴ is H, halo, C₁₋₆ alkyl, C₂₋₆ alkenyl, C₂₋₆ alkynyl, C₁₋₆ haloalkyl, C₆₋₁₀ aryl, C₆₋₁₀ aryl-C₁₋₄ alkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₁₋₆ haloalkyl, C₆₋₁₀ aryl, and C₆₋₁₀ aryl-C₁₋₄ alkyl, of R³ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
wherein at least one of R³ and R⁴ is other than H;
each R⁵ and R⁶ is independently selected from H, halo, C₁₋₆ alkyl, C₁₋₆ haloalkyl, CN, NO₂, OR^{a5}, and SR^{a5};
R⁷ is a group having the formula: —(C₁₋₂ alkyl)ₐ-(L¹)_{b}-(C₂₋₆ alkyl)_{c}-(L²)_{d}-Q;
L is —O—, —S—, —NR⁸—, —CO—, —SO—, —SO₂—, —SO₂NR⁸—, or —SONR⁸—;
L² is —O—, —S—, —NR¹⁰—, —CO—, —SO—, —SO₂—, —SO₂NR⁸—, or —SONR¹⁰—;
R⁸, R⁹, R¹⁰, and R¹¹ are each independently selected from H and C₁₋₄ alkyl;
a is 0 or 1;
b is 0 or 1;
c is 0 or 1;
d is 0 or 1;
wherein the sum of b and d is 1 or 2;
wherein the sum of a and c is 1 or 2;
Q is 5-7 membered heterocycloalkyl, each optionally substituted by 1, 2, 3 or 4 substituents selected from halo, C₁₋₆ alkyl, C₁₋₆ haloalkyl, CN, NO₂, OR^{a1}, SR^{a}, C(O)R^{b}, C(O)NR^{c}R^{d}, C(O)OR^{a1}, OC(O)R^{b}, and OC(O)NR^{c}R^{d};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR, SR, C(O)R^{b2}, C(O)NR^{c2}R^{d2}, C(O)OR, OC(O)R^{b2}, OC(O)NR^{c2}R^{d2}, NR^{c2}R^{d2}, NR^{c2}C(O)R^{b2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, S(O)R^{b3}, S(O)NR^{c3}R^{d3}, S(O)₂R^{b3}, and S(O)₂NR^{c3}R^{d3};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, S(O)₂NR^{c6}R^{d6}, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, R^{d3} R^{a4}, R^{b4}, R^{c4}, and R^{d4} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a6}SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)₂R^{b6}, and S(O)₂NR^{c6}R^{d6};
each R^{a5}, R^{b5}, R^{c5}, and R^{d5} is independently selected from H and C₁₋₆ alkyl;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each Re, R^{e1}, R^{e2}, R^{e3}, R^{e4}, R^{e5}, and R^{e6} is independently selected from H, C₁₋₄ alkyl, and CN,
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group;
wherein one or more ring-forming S atoms of any aforementioned heterocycloalkyl group is optionally substituted by one or two oxo (═O) groups.

3. The compound of claim 1 or pharmaceutically acceptable salt thereof, wherein X is N.

4.-11. (canceled)

12. The compound of claim 1 or pharmaceutically acceptable salt thereof, wherein R¹ is the group having Formula (ii):

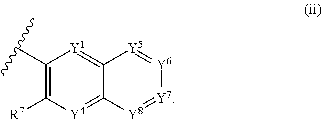

13. The compound of claim 12 or pharmaceutically acceptable salt thereof, wherein Y¹ is CR^{Y1}, Y⁴ is CR^{Y4}, Y⁵ is CR^{Y5}, Y⁶ is CR^{Y6}, Y⁷ is CR^{Y7}, and Y⁸ is CR^{Y8}.

14. The compound of claim 13 or pharmaceutically acceptable salt thereof, wherein R^{Y1}, R^{Y4}, R^{Y5}, R^{Y6}, R^{Y7}, and R^{Y8} are each independently selected from H, halo, C₁₋₆ alkyl, C₁₋₆ haloalkyl, CN, NO₂, and OR^{a1}.

15.-34. (canceled)

35. The compound of claim 1 or pharmaceutically acceptable salt thereof, wherein R³ is H or NR^{c3}C(S)NR^{c3}R^{d3}.

36.-37. (canceled)

38. The compound of claim 1 or pharmaceutically acceptable salt thereof, wherein each R^{c3} is selected from H and C₆₋₁₀ aryl, wherein said C₆₋₁₀ aryl is optionally substituted with 1, 2, 3, 4, or 5 substituents independently selected from halo, C₁₋₄ alkyl, C₁₋₆ haloalkyl, CN, OR^{a6}, and NR^{c6}R^{d6}.

39.-41. (canceled)

42. The compound of claim 1 or pharmaceutically acceptable salt thereof, wherein R⁴ is H, halo, C₁₋₆ alkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a4}, C(O)NR^{c4}R^{d4}, S(O)₂R^{b4}, C₆₋₁₀ aryl-C₁₋₄ alkyl, NO₂, NR^{c4}R^{d4}, NR^{c4}C(O)R^{b4}, NR^{c4}S(O)₂R^{b4}, or C(O)OR^{a4}.

43.-45. (canceled)

46. The compound of claim 1 or pharmaceutically acceptable salt thereof, wherein each R^{a4} is H or C₁₋₆ alkyl.

47. (canceled)

48. The compound of claim 1 or pharmaceutically acceptable salt thereof, wherein each R^{b4} is independently selected from H, halo, C₁₋₆ alkyl, C₁₋₆ haloalkyl, C₃₋₇ cycloalkyl, 4-10 membered heterocycloalkyl, OR^{a6}, NR^{c6}R^{d6}, SR^{a6}, wherein said C₁₋₆ alkyl, C₃₋₇ cycloalkyl, and 4-10 membered heterocycloalkyl is optionally substituted with halo, C₁₋₄ alkyl, C₁₋₄ haloalkyl, OR^{a6}, SR^{a6}, or NR^{c6}R^{d6}.

49. The compound of claim 1 or pharmaceutically acceptable salt thereof, wherein each R^{b4} is methyl, N(CH₃)₂, SCHF₂, OCH₂CF₃, phenyl, morpholinyl, cyclohexyl, 2-oxa-6-azaspiro[3.3]heptanyl, pyrrolidinyl, azetidinyl, or piperidinyl; wherein said morpholinyl, cyclohexyl, pyrrolidinyl, azetidinyl, and piperidinyl are each optionally substituted with OR^{a6}.

50.-51. (canceled)

52. The compound of claim 1 or pharmaceutically acceptable salt thereof, wherein R^{d4} is H, C₁₋₆ alkyl, C₁₋₆ haloalkyl, C₃₋₇ cycloalkyl, C₆₋₁₀ aryl, 5-10 membered heteroaryl, wherein each R^{d4} is optionally substituted with 1, 2, 3, 4, or 5 substituents independently selected from halo, C₁₋₄ alkyl, C₁₋₄ haloalkyl, and C₁₋₆ haloalkyl.

53. The compound of claim 52 or pharmaceutically acceptable salt thereof, wherein R^{d4} is H, methyl, cyclopropyl, or pyridinyl.

54. The compound of claim 1 or pharmaceutically acceptable salt thereof, wherein R² is H or R⁵ is H or R⁶ is H.

55.-56. (canceled)

57. The compound of claim 1 or pharmaceutically acceptable salt thereof, wherein a is 0 or b is 1 or c is 1 or d is 0.

58.-61. (canceled)

62. The compound of claim 1 or pharmaceutically acceptable salt thereof, wherein L¹ is —O—, —NR⁸—, —CO—, —C(O)O—, or —CONR⁸—.

63. (canceled)

64. The compound of claim 1 or pharmaceutically acceptable salt thereof, wherein Q is 5-7 membered heterocycloalkyl, optionally substituted by 1, 2, 3 or 4 substituents selected from halo, C₁₋₆ alkyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, C(O)R^{b}, C(O)NR^{c}R^{d}, C(O)OR^{a}, OC(O)R^{b}, NR^{c}R^{d}, NR^{c}C(O)R^{b}, S(O)R^{b}, S(O)NR^{c}R^{d}, S(O)₂R^{b}, and S(O)₂NR^{c}R^{d}.

65.-66. (canceled)

67. The compound of claim 1 or pharmaceutically acceptable salt thereof, wherein R⁷ is a group having the formula: 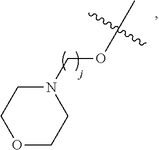
wherein j is 2, 3, 4, 5, or 6.

68. The compound of claim 1 or pharmaceutically acceptable salt thereof, having the formula: 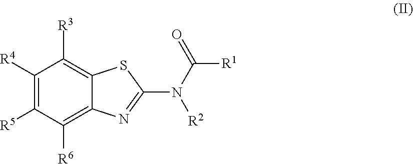
or pharmaceutically acceptable salt thereof, or 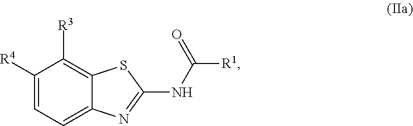
or pharmaceutically acceptable salt thereof, or 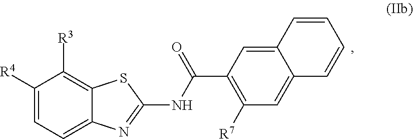
or pharmaceutically acceptable salt thereof, or 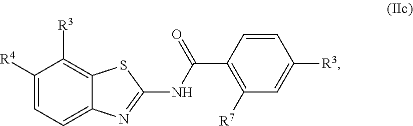
or pharmaceutically acceptable salt thereof, or 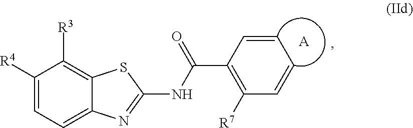
or pharmaceutically acceptable salt thereof, or 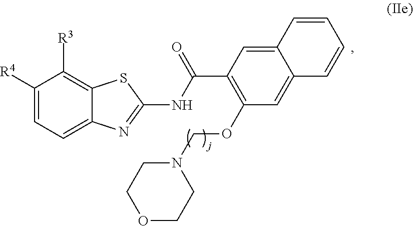
wherein j is 2, 3, 4, 5 or 6, or pharmaceutically acceptable salt thereof, or 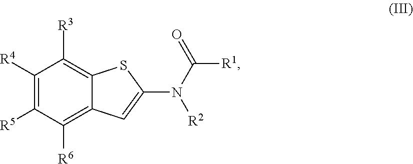
or pharmaceutically acceptable salt thereof, or 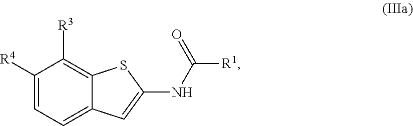
or pharmaceutically acceptable salt thereof, or 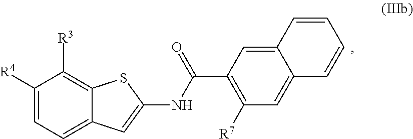
or pharmaceutically acceptable salt thereof, or 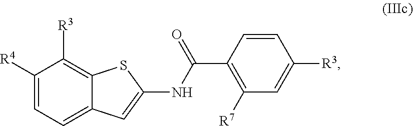
or pharmaceutically acceptable salt thereof, or 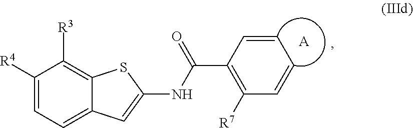
or pharmaceutically acceptable salt thereof, or 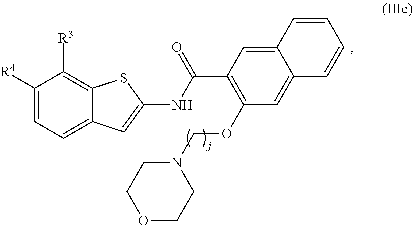
wherein j is 2, 3, 4, 5, or 6 or pharmaceutically acceptable salt thereof.

69.-79. (canceled)

80. The compound of claim 1, wherein the compound is selected from:
2-{3-[2-(morpholin-4-yl)ethoxy]naphthalene-2-amido}-N-(pyridin-2-yl)-1,3-benzothiazole-6-carboxamide;
2-{3-[4-(morpholin-4-yl)butoxy]naphthalene-2-amido}-N-(pyridin-2-yl)-1,3-benzothiazole-6-carboxamide;
3-[2-(morpholin-4-yl)ethoxy]-N-[6-(morpholine-4-sulfonyl)-1,3-benzothiazol-2-yl]naphthalene-2-carboxamide;
3-[4-(morpholin-4-yl)butoxy]-N-[6-(morpholine-4-sulfonyl)-1,3-benzothiazol-2-yl]naphthalene-2-carboxamide;
N-(6-benzyl-1,3-benzothiazol-2-yl)-3-[2-(morpholin-4-yl)ethoxy]naphthalene-2-carboxamide;
N-(2,6-dichlorophenyl)-2-{3-[2-(morpholin-4-yl)ethoxy]naphthalene-2-amido}-1,3-benzothiazole-6-carboxamide;
3-[2-(morpholin-4-yl)ethoxy]-N-(6-nitro-1,3-benzothiazol-2-yl)naphthalene-2-carboxamide;
N-(6-cyclohexaneamido-1,3-benzothiazol-2-yl)-3-[2-(morpholin-4-yl)ethoxy]naphthalene-2-carboxamide;
3-[4-(morpholin-4-yl)butoxy]-N-(6-nitro-1,3-benzothiazol-2-yl)naphthalene-2-carboxamide;
N-(6-cyano-1,3-benzothiazol-2-yl)-3-[2-(morpholin-4-yl)ethoxy]naphthalene-2-carboxamide;
N-(6-cyano-1,3-benzothiazol-2-yl)-3-[4-(morpholin-4-yl)butoxy]naphthalene-2-carboxamide;
3-[2-(morpholin-4-yl)ethoxy]-N-{7-[(phenylcarbamothioyl) amino]-1,3-benzothiazol-2-yl}naphthalene-2-carboxamide;
N-(6-methanesulfonamido-1,3-benzothiazol-2-yl)-3-[4-(morpholin-4-yl)butoxy]naphthalene-2-carboxamide;
3-[2-(morpholin-4-yl)ethoxy]-N-[6-(pyrrolidine-1-sulfonyl)-1,3-benzothiazol-2-yl]naphthalene-2-carboxamide;
N-[6-(dimethylsulfamoyl)-1,3-benzothiazol-2-yl]-3-[2-(morpholin-4-yl)ethoxy]naphthalene-2-carboxamide;
3-[4-(morpholin-4-yl)butoxy]-N-[6-(pyrrolidine-1-sulfonyl)-1,3-benzothiazol-2-yl]naphthalene-2-carboxamide;
3-[4-(morpholin-4-yl)butoxy]-N-(7-{[(pyridin-2-yl) carbamothioyl]amino}-1,3-benzothiazol-2-yl) naphthalene-2-carboxamide;
3-[2-(morpholin-4-yl)ethoxy]-N-(7-{[(pyridin-2-yl)carbamothioyl] amino}-1,3-benzothiazol-2-yl)naphthalene-2-carboxamide;
N-[6-(azetidine-1-sulfonyl)-1,3-benzothiazol-2-yl]-3-[2-(morpholin-4-yl)ethoxy]naphthalene-2-carboxamide;
N-[6-(azetidine-1-sulfonyl)-1,3-benzothiazol-2-yl]-3-[4-(morpholin-4-yl)butoxy]naphthalene-2-carboxamide;
3-[2-(morpholin-4-yl)ethoxy]-N-[6-(piperidine-1-sulfonyl)-1,3-benzothiazol-2-yl]naphthalene-2-carboxamide;
3-[4-(morpholin-4-yl)butoxy]-N-[6-(piperidine-1-sulfonyl)-1,3-benzothiazol-2-yl]naphthalene-2-carboxamide;
N-{6-[(difluoromethyl)sulfanyl]-1,3-benzothiazol-2-yl}-3-[2-(morpholin-4-yl) ethoxy]naphthalene-2-carboxamide;
N-{6-[(difluoromethyl) sulfanyl]-1,3-benzothiazol-2-yl}-3-[4-(morpholin-4-yl) butoxy]naphthalene-2-carboxamide;
3-[2-(morpholin-4-yl) ethoxy]-N-[6-(pyrrolidine-1-sulfonyl)-1,3-benzothiazol-2-yl]-[1,1′-biphenyl]-4-carboxamide;
3-[4-(morpholin-4-yl)butoxy]-N-[6-(pyrrolidine-1-sulfonyl)-1,3-benzothiazol-2-yl]-[1,1′-biphenyl]-4-carboxamide;
6-[2-(morpholin-4-yl)ethoxy]-N-[6-(pyrrolidine-1-sulfonyl)-1,3-benzothiazol-2-yl]-1-benzothiophene-5-carboxamide;
6-[4-(morpholin-4-yl)butoxy]-N-[6-(pyrrolidine-1-sulfonyl)-1,3-benzothiazol-2-yl]-1-benzothiophene-5-carboxamide;
3-[2-(morpholin-4-yl)ethoxy]-N-[6-(2,2,2-trifluoroethoxy)-1,3-benzothiazol-2-yl]naphthalene-2-carboxamide;
3-[4-(morpholin-4-yl)butoxy]-N-(6-{2-oxa-6-azaspiro[3.3]heptane-6-sulfonyl}-1,3-benzothiazol-2-yl)naphthalene-2-carboxamide;
N-{6-[(3-hydroxypyrrolidin-1-yl)sulfonyl]-1,3-benzothiazol-2-yl}-3-[2-(morpholin-4-yl)ethoxy]naphthalene-2-carboxamide;
6-[4-(morpholin-4-yl)butoxy]-N-[6-(pyrrolidine-1-sulfonyl)-1,3-benzothiazol-2-yl]-2H-1,3-benzodioxole-5-carboxamide;
6-[2-(morpholin-4-yl)ethoxy]-N-[6-(pyrrolidine-1-sulfonyl)-1,3-benzothiazol-2-yl]-2H-1,3-benzodioxole-5-carboxamide;
methyl 2-{3-[4-(morpholin-4-yl)butoxy]naphthalene-2-amido}-1,3-benzothiazole-6-carboxylate;
1-methyl-5-[2-(morpholin-4-yl)ethoxy]-N-[6-(pyrrolidine-1-sulfonyl)-1,3-benzothiazol-2-yl]-1H-indole-6-carboxamide;
methyl 2-{3-[2-(morpholin-4-yl)ethoxy]naphthalene-2-amido}-1,3-benzothiazole-6-carboxylate;
N-(6-{[(3R)-3-hydroxypyrrolidin-1-yl]sulfonyl}-1,3-benzothiazol-2-yl)-3-[2-(morpholin-4-yl)ethoxy]naphthalene-2-carboxamide;
N-(6-{[(3R)-3-hydroxypyrrolidin-1-yl]sulfonyl}-1,3-benzothiazol-2-yl)-3-[4-(morpholin-4-yl)butoxy]naphthalene-2-carboxamide;
3-[4-(morpholin-4-yl)butoxy]-N-[6-(pyrrolidine-1-sulfonyl)-1-benzothiophen-2-yl]-[1,1′-biphenyl]-4-carboxamide;
3-[4-(morpholin-4-yl)butoxy]-N-[6-(pyrrolidine-1-sulfonyl)-1-benzothiophen-2-yl]naphthalene-2-carboxamide;
3-[2-(morpholin-4-yl)ethoxy]-N-[6-(pyrrolidine-1-sulfonyl)-1-benzothiophen-2-yl]-[1,1′-biphenyl]-4-carboxamide;
6-[4-(morpholin-4-yl)butoxy]-N-[6-(pyrrolidine-1-sulfonyl)-1-benzothiophen-2-yl]-1-benzothiophene-5-carboxamide;
6-[4-(morpholin-4-yl)butoxy]-N-[6-(pyrrolidine-1-sulfonyl)-1-benzothiophen-2-yl]-2H-1,3-benzodioxole-5-carboxamide;
3-[2-(morpholin-4-yl)ethoxy]-N-[6-(pyrrolidine-1-sulfonyl)-1-benzothiophen-2-yl]naphthalene-2-carboxamide;
3-[2-(morpholin-4-yl)ethoxy]-N-[6-(pyrrolidine-1-sulfonyl)-1-benzothiophen-2-yl]naphthalene-2-carboxamide; and
6-[2-(morpholin-4-yl)ethoxy]-N-[6-(pyrrolidine-1-sulfonyl)-1-benzothiophen-2-yl]-1-benzothiophene-5-carboxamide; and
3-[2-(morpholin-4-yl)ethoxy]-N-[6-(pyrrolidine-1-sulfonyl)-1,3-benzothiazol-2-yl]-1-benzothiophene-2-carboxamide;
[N-(6-(azetidin-1-ylsulfonyl)benzo[b]thiophen-2-yl)-3-(4-morpholinobutoxy)-2-naphthamide];
N-(6-(((2R,5S)-2,5-dimethylpyrrolidin-1-yl) sulfonyl)benzo[d]thiazol-2-yl)-3-(4-morpholinobutoxy)-2-naphthamide;
3-{2-[2-(methoxymethyl)morpholin-4-yl]ethoxy}-N-[6-(pyrrolidine-1-sulfonyl)-1,3-benzothiazol-2-yl]naphthalene-2-carboxamide;
3-(2-{2-oxa-5-azabicyclo[2.2.2]octan-5-yl}ethoxy)-N-[6-(pyrrolidine-1-sulfonyl)-1,3-benzothiazol-2-yl]naphthalene-2-carboxamide;
3-(2-{6-oxa-3-azabicyclo[3.1.1]heptan-3-yl}ethoxy)-N-[6-(pyrrolidine-1-sulfonyl)-1,3-benzothiazol-2-yl]naphthalene-2-carboxamide;
3-{2-[(1R,4R)-2-oxa-5-azabicyclo[2.2.1]heptan-5-yl]ethoxy}-N-[6-(pyrrolidine-1-sulfonyl)-1,3-benzothiazol-2-yl]naphthalene-2-carboxamide;
6-[2-(morpholin-4-yl)ethoxy]-N-[6-(pyrrolidine-1-sulfonyl)-1,3-benzothiazol-2-yl]-2,3-dihydro-1H-indene-5-carboxamide
or a pharmaceutically acceptable salt thereof.

81. A compound selected from:
N-{6-[(difluoromethyl)sulfanyl]-1,3-benzothiazol-2-yl}naphthalene-2-carboxamide;
N-[6-(2,2,2-trifluoroethoxy)-1,3-benzothiazol-2-yl]naphthalene-2-carboxamide;
N-(6-methanesulfonamido-1,3-benzothiazol-2-yl)naphthalene-2-carboxamide;
N-(6-cyclohexaneamido-1,3-benzothiazol-2-yl)naphthalene-2-carboxamide;
N-{6-[(trifluoromethyl) sulfanyl]-1,3-benzothiazol-2-yl}naphthalene-2-carboxamide;
N-[6-(1H-1,3-benzodiazol-2-yl)-1,3-benzothiazol-2-yl]naphthalene-2-carboxamide;
N-[7-(trifluoromethyl)-1,3-benzothiazol-2-yl]naphthalene-2-carboxamide;
N-{6-[(cyclopropylcarbamoyl) methyl]-1,3-benzothiazol-2-yl}naphthalene-2-carboxamide;
N-(2,6-dichlorophenyl)-2-(naphthalene-2-amido)-1,3-benzothiazole-6-carboxamide;
2-(naphthalene-2-amido)-N-(pyridin-2-yl)-1,3-benzothiazole-6-carboxamide;
N-{6-[(2,6-dichlorophenyl)carbamoyl]-1,3-benzothiazol-2-yl}quinoline-6-carboxamide;
N-[6-(azetidine-1-sulfonyl)-1,3-benzothiazol-2-yl]-[1,1′-biphenyl]-4-carboxamide;
N-[6-(azetidine-1-sulfonyl)-1,3-benzothiazol-2-yl]-1-methyl-1H-indole-6-carboxamide;
N-[6-(azetidine-1-sulfonyl)-1,3-benzothiazol-2-yl]-1-methyl-1H-indole-2-carboxamide;
N-[6-(azetidine-1-sulfonyl)-1,3-benzothiazol-2-yl]-1-benzothiophene-2-carboxamide;
1-methyl-N-[6-(pyrrolidine-1-sulfonyl)-1,3-benzothiazol-2-yl]-1H-indole-2-carboxamide;
3-[2-(morpholin-4-yl)ethoxy]-N-[6-(pyrrolidine-1-sulfonyl)-1,3-benzothiazol-2-yl]-1-benzothiophene-2-carboxamide
3-[2-(4,4-difluoropiperidin-1-yl)ethoxy]-N-[6-(pyrrolidine-1-sulfonyl)-1,3-benzothiazol-2-yl]naphthalene-2-carboxamide;
3-[4-(4,4-difluoropiperidin-1-yl)butoxy]-N-[6-(pyrrolidine-1-sulfonyl)-1,3-benzothiazol-2-yl]naphthalene-2-carboxamide;
N-(6-{[(3S)-3-hydroxypyrrolidin-1-yl]sulfonyl}-1,3-benzothiazol-2-yl)-3-[2-(morpholin-4-yl)ethoxy]naphthalene-2-carboxamide; and
N-(6-{[(3S)-3-hydroxypyrrolidin-1-yl]sulfonyl}-1,3-benzothiazol-2-yl)-3-[4-(morpholin-4-yl)butoxy]naphthalene-2-carboxamide;
[N-(6-(azetidin-1-ylsulfonyl)benzo[b]thiophen-2-yl)-3-(4-morpholinobutoxy)-2-naphthamide];
N-(6-(((2R,5S)-2,5-dimethylpyrrolidin-1-yl) sulfonyl)benzo[d]thiazol-2-yl)-3-(4-morpholinobutoxy)-2-naphthamide;
or a pharmaceutically acceptable salt thereof.

82. A pharmaceutical composition comprising a compound of claim 1, or a pharmaceutically acceptable salt thereof, and at least one pharmaceutically acceptable carrier.

83.-88. (canceled)

89. A method for treating cancer in a subject, said method comprising administering to the subject a therapeutically effective amount of the compound of claim 1, or a pharmaceutically acceptable salt thereof.

90.-91. (canceled)

92. A pharmaceutical composition comprising:
N-(6-acetamido-1,3-benzothiazol-2-yl) naphthalene-2-carboxamide;
N-(6-methanesulfonyl-1,3-benzothiazol-2-yl) naphthalene-2-carboxamide;
methyl 2-(naphthalene-2-amido)-1,3-benzothiazole-6-carboxylate;
N-[6-(dimethylsulfamoyl)-1,3-benzothiazol-2-yl] naphthalene-2-carboxamide;
N-(6-methyl-1,3-benzothiazol-2-yl) naphthalene-2-carboxamide;
N-[6-(morpholine-4-sulfonyl)-1,3-benzothiazol-2-yl]naphthalene-2-carboxamide;
N-[6-(piperidine-1-sulfonyl)-1,3-benzothiazol-2-yl]naphthalene-2-carboxamide;
N-(6-chloro-1,3-benzothiazol-2-yl)naphthalene-2-carboxamide;
N-(6-benzamido-1,3-benzothiazol-2-yl)naphthalene-2-carboxamide;
N-[2-(naphthalene-2-amido)-1,3-benzothiazol-6-yl]furan-2-carboxamide;
N-[6-(pyrrolidine-1-sulfonyl)-1,3-benzothiazol-2-yl]naphthalene-2-carboxamide; and
N-[6-(methylsulfamoyl)-1,3-benzothiazol-2-yl]naphthalene-2-carboxamide;
[N-(6-(azetidin-1-ylsulfonyl)benzo[b]thiophen-2-yl)-3-(4-morpholinobutoxy)-2-naphthamide];
N-(6-(((2R,5S)-2,5-dimethylpyrrolidin-1-yl) sulfonyl)benzo[d]thiazol-2-yl)-3-(4-morpholinobutoxy)-2-naphthamide;
or a pharmaceutically acceptable salt thereof, and at least one pharmaceutically acceptable carrier.

93.-101. (canceled)
